(19) United States
(12) Patent Application Publication
(10) Pub. No.: US 2022/0315609 A1
(43) Pub. Date: Oct. 6, 2022

(54) NOVEL LUMINESCENT LANTHANIDE CHELATE REPORTERS, BIOSPECIFIC BINDING REACTANTS LABELLED WITH NOVEL LUMINESCENT LANTHANIDE CHELATE REPORTERS AND THEIR USE

(71) Applicant: RADIOMETER TURKU OY, Turku (FI)

(72) Inventors: Henri SUND, Turku (FI); Harri TAKALO, Turku (FI)

(21) Appl. No.: 17/596,841

(22) PCT Filed: Jun. 24, 2020
(86) PCT No.: PCT/EP2020/067685

§ 371 Date: Dec. 20, 2021

(30) Foreign Application Priority Data
Jun. 25, 2019 (DK) PA 2019 00772

(51) Int. Cl. C07F 5/00; G01N 33/58

(52) U.S. Cl. CPC C07F 5/003; G01N 33/582

(57) ABSTRACT

The present invention relates to novel luminescent lanthanide chelate reporters which are formed from two to three separate lanthanide chelate moieties covalently conjugated to each other to act as an unique labelling reactant, and which can be attached to a biospecific reactant and used in various assays.

### TECHNICAL FIELD

The present invention relates to novel luminescent lanthanide chelate reporters which are formed from two to three separate lanthanide chelate moieties covalently conjugated to each other to act as an unique labelling reactant, and which can be attached to a biospecific reactant and used in various assays.

### BACKGROUND OF THE INVENTION

Time-resolved fluorometry (TRF) employing long-lifetime emitting luminescent lanthanide chelates has been applied in many specific binding assays, such as e.g. immunoassays, DNA hybridization assays, receptor-binding assays, enzymatic assays, bio-imaging such as immunocytochemical, immunohistochemical assays or cell based assays to measure wanted analyte at very low concentration. Moreover, lanthanide chelates have been used in magnetic resonance imaging (MRI) and position emission tomography (PET).

For TRF application, an optimal label has to fulfil several requirements. First, it has to be photochemically stable both in the ground state and in the excited state and it has to be kinetically and chemically stable. The excitation wavelength has to be as high as possible, preferable over 300 nm. It has to have efficient cation emission i.e. brightness (excitation coefficient×quantum yield, εΦ). The observed luminescence decay time has to be long, and the chelate has to have good water solubility. For the purpose of labelling, it should have a reactive group to allow covalent attachment to a biospecific binding reactant, and the affinity and nonspecific binding properties of the labelled biomolecules have to be retained.

Since the publication of label chelates which contain one to three separate 4-(phenylethynyl)pyridines (U.S. Pat. No. 4,920,195; Takalo, H., et al., Helv. Chim. Acta. 79(1996)789), the designed ligand structures have been applied in many patents, patent applications and publications. One generally used method to improve luminescence intensity i.e. brightness is to enhance chelate's molar absorptivity by having several independent chromophore moieties i.e. 4-(phenylethynyl)pyridines combined in structure designs, which offer high stabilities and luminescence quantum yields (see e.g. WO 2013/026790; WO 2013/092992; WO 2016/066641). It is generally known that the luminescence intensity is improved also by increasing chromophore's molar absorptivity together with quantum yield. The molar absorptivity can be enhanced by increasing the π-electron conjugation of the aromatic chromophore (see e.g. WO 2015/165826).

Although the disclosed labels can offer high sensitive assays, antibodies used in assays can suffer from the high labelling degree (i.e. amount of chelates per antibody (Ab) or biomolecule). It is generally known, that the assay sensitivity can be increased by increasing the amount of labels in biomolecule such as IgG. Normally, too high labelling degree means more aggregates during the Ab labelling, and thus, causes purification problems of the labelled Ab. Moreover, antibody's affinity is reduced and back-ground is increased. Therefore, the labelling degree with most of Abs have to be optimized and practically cannot be over 15-20 Eu/IgG depending on the Ab in question. Moreover, certain Abs or biomolecules (such as oligopeptides or oligonucleotides) do not contain several functional groups (such as primary amino groups) to be used for labelling and/or do not tolerate several labels per Ab. Related to oligopeptides and oligonucleotides the problem of adequate amount of labels per biomolecule has been solved by using solid-phase labelling, which allow introduction of several chelates to oligopeptides and oligonucleotides (see e.g. Hovinen, J., et al., Bioconjugate Chem. 20(2009)404). However, the disclosed labelling method and such solid-phase prepared labels cannot be used for normal biomolecule labelling (e.g. for Abs) in aqueous solutions. Also polymers or dendrimers as a backbone for several chelates could offer a possibility to increase the amount of chelates per biomolecule. However, the conjugation of biomolecules to such bulky polymeric molecules with chelates causes purification problems and unwanted unspecific reactions, and thus, destroys the functionality of biomolecule to be labelled. Therefore, the use of disclosed polymeric or dendrimeric chelate labels have been constricted to imaging applications such as MRI (see e.g. Andolina, C. M., et. al, Macromolecules, 45(2012)8982) and cannot be practically used for specific labelling of biomolecules such as Abs.

Against this background, it is an object of the present invention to provide luminescent lanthanide chelate reporters for labelling biospecific binding reactants such as antibodies, which provide an increased brightness of the labelled biospecific binding reactants without reducing the its affinity.

It is a further object of the present invention to provide luminescent lanthanide chelate reporters for labelling biospecific binding reactants such as antibodies, which provide an increased brightness of the labelled biospecific binding reactants without causing purification problems of the labelled biospecific binding reactants.

It is yet a further object of the present invention to provide luminescent lanthanide chelate reporters for labelling biospecific binding reactants such as antibodies, which are suitable for labelling biospecific binding reactants with only few functional groups (such as primary amino groups) to be used for labelling.

### SUMMARY OF THE INVENTION

It has been found that the above objects are solved by luminescent lanthanide chelate reporters comprising two or three separate lanthanide chelate moieties, which are covalently tethered to each other.

In one aspect, the present invention therefore relates to a compound of formula (I)

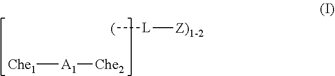

or a salt thereof, wherein
(i) the solid lines represent covalent bonds;
(ii) the dashed line represents a covalent bond of the group -L-Z to any one of the groups Che₁, A₁, and Che₂;
   and wherein
L is in each case independently absent or selected from linker groups comprising from 1 to 10 moieties selected from —(CH₂)₁₋₈—, —CH═CH—, —C≡C—, —O—, —S—, —S—S—, —C(═O)—, —C(═O)NH—, —NHC(═O)—, —C(═O)N(C₁-C₆-alkyl)-, —N(C₁-C₆-alkyl)C(═O)—, —NHC(═S)NH—, —CH[(CH₂)₀₋₆C(═O)O⁻]—, —CH[(CH₂)₀₋₆C(═O)OH]—, and biradicals of 5- to 10-membered aromatic or heteroaromatic monocyclic or bicyclic rings, wherein the heteroaromatic ring contains one or more, same or different heteroatoms N, O, or S;
Z is in each case independently selected from reactive groups selected from —N₃, —C≡CH, —CH═CH₂, —NH₂, —O—NH₂, —C(═O)OH, —CH(═O), —SH, —OH, maleimido and activated derivatives thereof including —NCO, —NCS, —N⁺≡N, bromoacetamido, iodoacetamido, reactive esters, pyridyl-2-dithio, and 6-substituted 4-chloro-1,3,5-triazin-2-ylamino and 4-chloro-1,3,5-triazin-2-yloxy; wherein the substituent in the 6-position of the 4-chloro-1,3,5-triazin-2-ylamino or 4-chloro-1,3,5-triazin-2-yloxy is selected from —H, -halogen, —SH, —NH₂, —C₁-C₆-alkyl, —O(C₁-C₆-alkyl), —OAryl, —S(C₁-C₆-alkyl), —SAryl, —N(C₁-C₆-alkyl)₂, and N(Aryl)₂;
   wherein the carbon atoms of the aforementioned groups are unsubstituted or substituted by one or more substituents selected from —CN, -halogen, —SH, —C(═O)H, —C(═O)OH, C₁-C₆-alkyl, C₁-C₆-haloalkyl, —O(C₁-C₆-alkyl), —C(═O)(C₁-C₆-alkyl), —C(═O)O(C₁-C₆-alkyl), and phenyl;
A₁ is a bridging group comprising from one to three separate straight or branched, saturated or unsaturated carbon-based chains including from 1 to 12 carbon atoms, wherein the carbon-based chains are free of or comprise one to ten, same or different groups selected from —O—, —S—, —NH—, —NR₁—, —C(═O)NH—, —NHC(═O)—, —C(═O)NR₁—, —NR₁C(═O)— and —C(═O)—,
   or
A₁ is a bridging chelate moiety -Che₃- of the following general formula

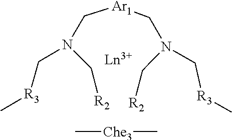

and wherein
Che₁ and Che₂ are independently selected from the chelate moieties Che I, Che II, Che III, Che IV, Che V, Che VI, and Che VII of the following general formulae:

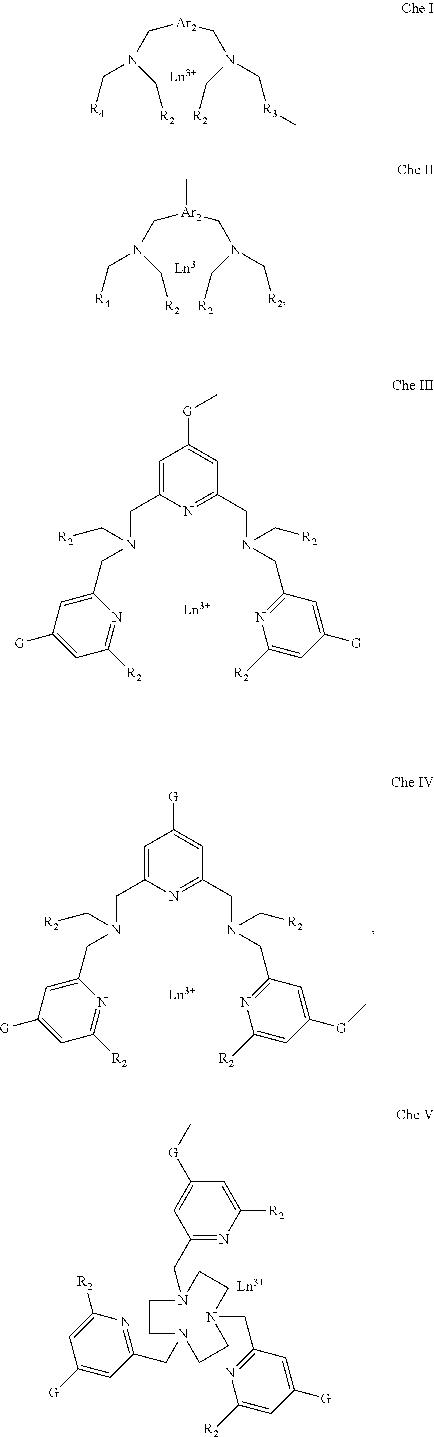

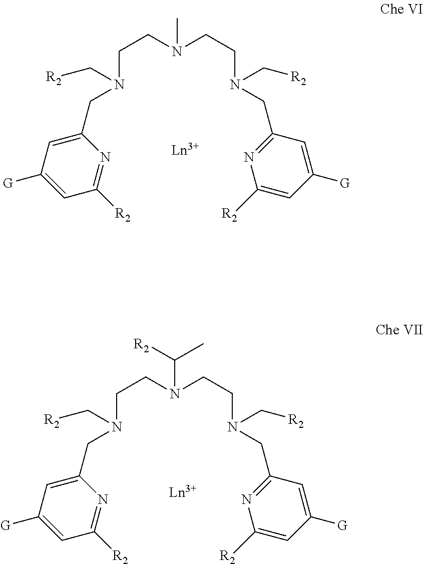

and wherein
R₁ is in each case independently selected from C₁-C₆-alkyl, and from the option of representing one of the one or two groups -L-Z;
R₂ is in each case independently selected from —C(═O)O⁻, —P(═O)O₂²⁻, P(═O)MeO⁻, —P(═O)PhO⁻, and the C₁-C₆-alkyl esters thereof, and from the option of representing one of the one or two groups -L-Z;
R₃ is in each case independently selected from the bridging groups —C(═O)NH—, —C(═O)NH—(C₁-C₆-alkylene)-NHC(═O)—, —C(═O)NH—(C₁-C₆-alkylene)-O—, —C(═O)NH—(C₁-C₆-alkylene)-C(═O)NH—, —C(═O)NH—(C₁-C₆-alkylene)-S—, —C(═O)NH—(C₁-C₆-alkylene)-NHC(═O)—(C₁-C₆-alkylene)-NHC(═O)—, —C(═O)NH—(C₁-C₆-alkylene)-NHC(═O)—(C₁-C₆-alkylene)-O—, —C(═O)NH—(C₁-C₆-alkylene)-NHC(═O)—(C₁-C₆-alkylene)-C(═O)NH—, —C(═O)NH—(C₁-C₆-alkylene)-NHC(═O)—(C₁-C₆-alkylene)-S—, —C(═O)NH—(C₁-C₆-alkylene)-O—(C₁-C₆-alkylene)-NHC(═O)—, —C(═O)NH—(C₁-C₆-alkylene)-O—(C₁-C₆-alkylene)-O—, —C(═O)NH—(C₁-C₆-alkylene)-O—(C₁-C₆-alkylene)-S—, —C(═O)NH—(C₁-C₆-alkylene)-O—(C₁-C₆-alkylene)-C(═O)NH—, —C(═O)NH—(C₁-C₆-alkylene)-C(═O)NH—(C₁-C₆-alkylene)-NHC(═O)—, —C(═O)NH—(C₁-C₆-alkylene)-C(═O)NH—(C₁-C₆-alkylene)-O—, —C(═O)NH—(C₁-C₆-alkylene)-C(═O)NH—(C₁-C₆-alkylene)-S—, —C(═O)NH—(C₁-C₆-alkylene)-C(═O)NH—(C₁-C₆-alkylene)-C(═O)NH—, and —P(═O)O₂²⁻;
R₄ is in each case independently selected from —CH₂N(CH₂C(═O)O⁻)₂, —CH₂N(CH₂P(═O)O₂²⁻)₂, —CH₂N(CH₂P(═O)MeO⁻)₂, —CH₂N(CH₂P(═O)PhO⁻)₂, and from the options defined for R₂;
Ln³⁺ is in each case independently selected from the lanthanide ions Eu³⁺, Tb³⁺, Sm³⁺ and Dy³⁺, wherein the lanthanide ion forms from seven to ten coordination bonds with the heteroatoms oxygen and nitrogen in the chelate moieties Che₁, Che₂, and Che₃ to form from two to three separate internal chelate moieties;
Ar₁ is selected from the following groups

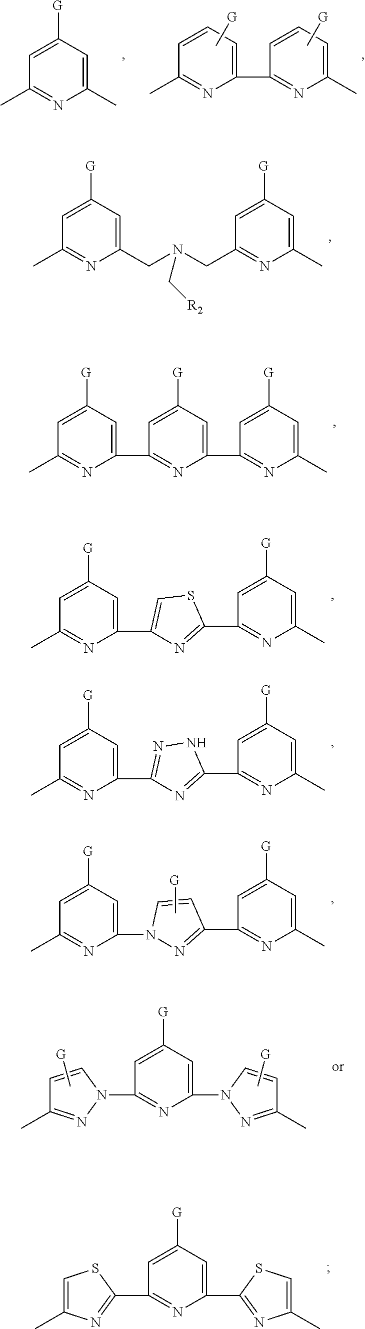

Ar₂ is in each case independently selected from the following groups

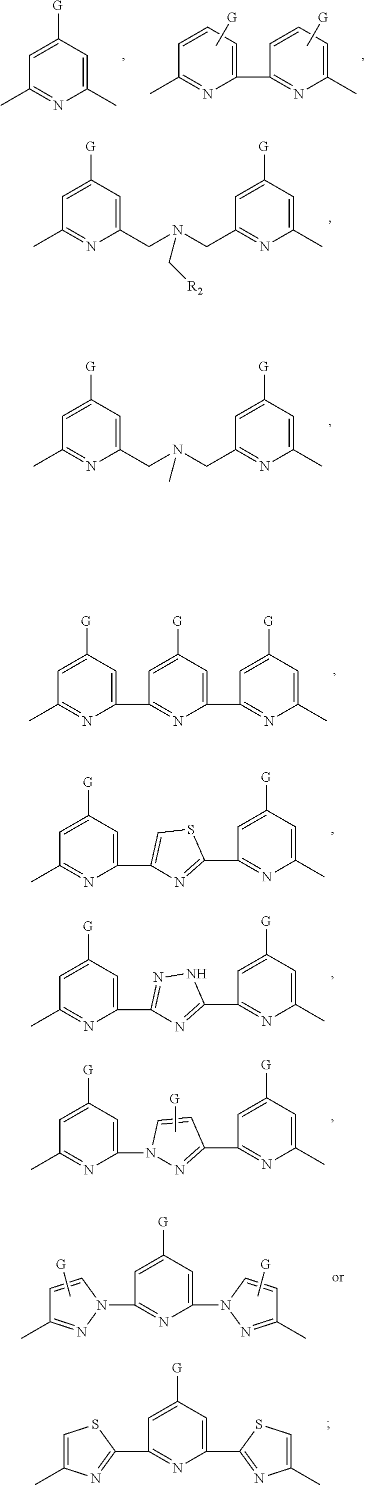

and wherein
G is in each case independently selected from i) a conjugating group, ii) a single bond, and iii) hydrogen;
   wherein each conjugating group comprises 1, 2, or 3 moieties selected from —CH═CH—, —C≡C—, —C(═O)—, and biradicals of 5 to 10-membered aromatic or heteroaromatic monocyclic or bicyclic rings, wherein the heteroaromatic ring contains one or more, same or different heteroatoms N, O, or S, and wherein the aromatic or heteroaromatic monocyclic or bicyclic rings are unsubstituted or substituted by 1 to 5 same or different substituents R₅;
   wherein each conjugating group, if present in a terminal position, may further comprise a terminal group, which is selected from the group consisting of —H, -halogen, —CN, —CH₃, and from the option of representing one of the one or two groups -L-Z.
   wherein R₅ is independently selected from C₁-C₁₂-alkyl, —(CH₂)₀₋₆—C(═O)OH, —(CH₂)₀₋₆—C(═O)O⁻, —(CH₂)₀₋₆—S(═O)₂OH, —(CH₂)₀₋₆—S(═O)₂O⁻, —C(═O)NHR₆, —C(═O)NCH₃R₆, —NHC(═O)NHR₆, —NHC(═S)NHR₆, -halogen, —OH, —SH, —OR₇, —SR₇, and hydrophilic groups selected from monosaccharides, disaccharides, —(CH₂)₁₋₆ CH₂OH, —CH(CH₂OH)₂, —C(CH₂OH)₃, —(CH₂)₁₋₃—O—(CH₂CH₂O)₀₋₅—H, —(CH₂)₁₋₃—O—(CH₂CH₂O)₀₋₅—C₁-C₄-alkyl, —O—(CH₂CH₂O)₁₋₆—H, and —O—(CH₂CH₂O)₁₋₆—C₁-C₄-alkyl;
   wherein R₆ is selected from C₁-C₁₂-alkyl, —(CH₂)₁₋₆C(═O)OH, —(CH₂)₁₋₆C(═O)O⁻, —(CH₂)₁₋₆ S(═O)₂OH, —(CH₂)₁₋₆ S(═O)₂O⁻ and hydrophilic groups selected from monosaccharides, disaccharides, —(CH₂)₁₋₆CH₂OH, —CH(CH₂OH)₂, —C(CH₂OH)₃, —(CH₂)₁₋₃—O—(CH₂CH₂O)₀₋₅—H, —(CH₂)₁₋₃—O—(CH₂CH₂O)₀₋₅—C₁-C₄-alkyl, —O—(CH₂CH₂O)₁₋₆—H, and —O—(CH₂CH₂O)₁₋₆—C₁-C₄-alkyl;
   wherein R₇ is selected from —CF₃, —C₁-C₁₂-alkyl, —(CH₂)₁₋₆C(═O)OH, —(CH₂)₁₋₆C(═O)O⁻, —(CH₂)₁₋₆S(═O)₂OH, —(CH₂)₁₋₆S(═O)₂O⁻, —C(═O)NHR₆, —C(═O)NCH₃R₆, —NHC(═O)NHR₆, —NHC(═S)NHR₆, —(CH₂)₁₋₆N(CH₃)₂⁺—(CH₂)₁₋₆S(═O)₂O⁻, —(CH₂)₁₋₆C(═O)-(piparazin-1,4-diyl)-(CH₂)₁₋₆C(═O)OH, and hydrophilic groups selected from monosaccharides, disaccharides, —(CH₂)₁₋₆CH₂OH, —CH(CH₂OH)₂, —C(CH₂OH)₃, —(CH₂)₁₋₃—O—(CH₂CH₂O)₀₋₅—H, —(CH₂)₁₋₃—O—(CH₂CH₂O)₀₋₅—C₁-C₄-alkyl, —O—(CH₂CH₂O)₁₋₆—H, and —O—(CH₂CH₂O)₁₋₆—C₁-C₄-alkyl.

The compounds of formula (I) can be used to increase the brightness of a labelled biospecific binding reactant such as an antibody (Ab) without reducing its affinity. Moreover, the labelled Ab can be efficiently separated from the excess of labelling reagent and side compounds. The compounds of formula (I) contain 1-2 reactive groups for the Ab labelling. When two reactive groups are used, higher labelling degree is obtained and/or less labelling reactant can be used to get the appropriate labelling degree. If a compound of formula (I) with two reactive groups is used for labelling, the chelate label will be rigid and compact. Therefore, it decreases possible thermal movement and rotation of the reporter, and reduces thermal de-activation processes of excited reporters and can increase the luminescence i.e. the brightness of labelled biomolecule.

In a further aspect, the present invention relates to a compound of formula (II)

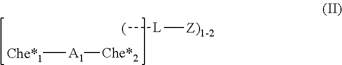

or a salt thereof, wherein

L, Z, R₁, R₂, R₃, R₄, Ar₁, Ar₂, G, R₅, R₆, and R₇ are defined as in connection with the compound of formula (I),

and wherein
A₁ is a bridging group comprising from one to three separate straight or branched, saturated or unsaturated carbon-based chains including from 1 to 12 carbon atoms, wherein the carbon-based chains are free of or comprise one to ten, same or different groups selected from —O—, —S—, —NH—, —NR₁—, —C(═O)NH—, —NHC(═O)—, —C(═O)NR₁—, —NR₁C(═O)— and —C(═O)—,
   or
A₁ a bridging chelate moiety -Che*₃- of the following general formula

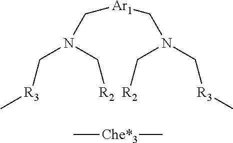

and wherein
Che*₁ and Che*₂ are independently selected from the chelate moieties Che* I, Che* II, Che* III, Che* IV, Che* V, Che* VI, and Che* VII of the following general formulae:

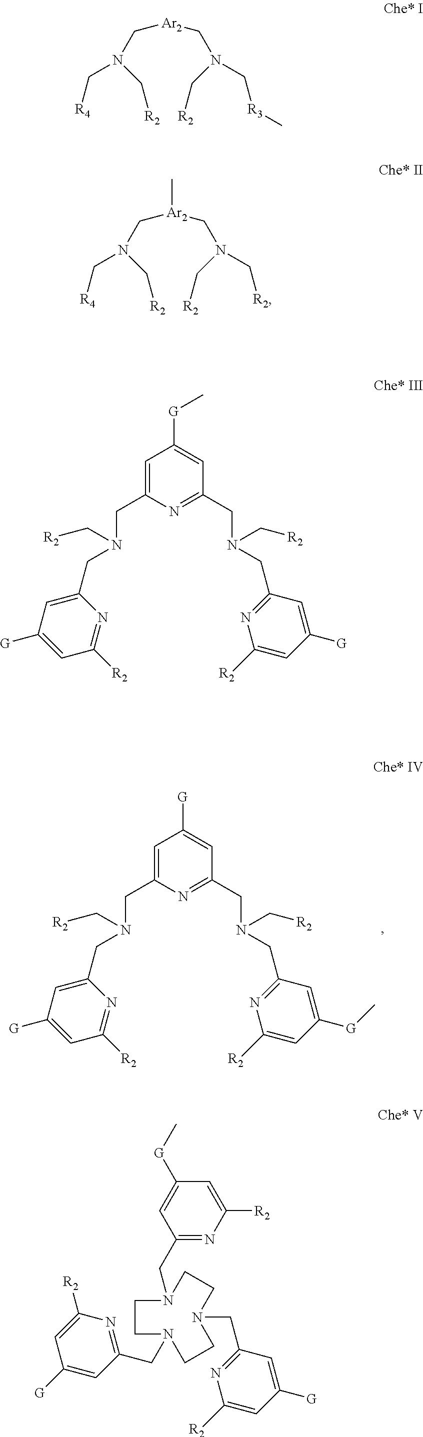

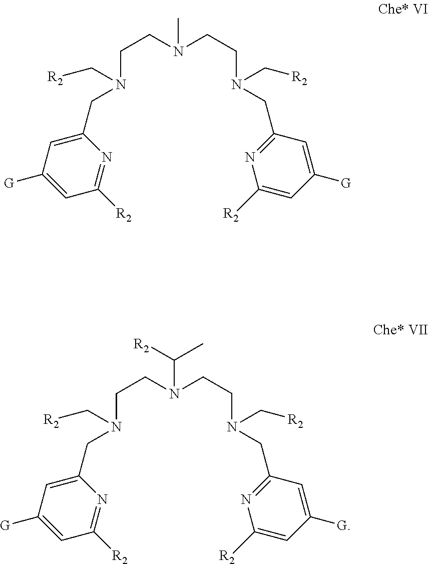

The compounds of formula (II) are precursor compounds for the luminescent lanthanide chelate reporters according to formula (I). The compounds of formula (I) may be obtained by the compounds of formula (II) by reacting the compounds of formula (II) with a lanthanide salt after deprotection of possible ester functions.

In a further aspect, the present invention relates to a detection agent comprising a biospecific binding reactant conjugated to a compound of formula (I) or (II) as defined above.

In yet a further aspect, the present invention relates to a method of detecting an analyte in a biospecific binding assay, said method comprising the steps of:

a) forming a complex between the analyte and a compound of formula (I) or (II) or a detection agent as defined above;
b) exciting said complex with a radiation having an excitation wavelength of the compound of formula (I) or the detection agent as defined above, thereby forming an excited complex; and
c) detecting emission radiation emitted from said excited complex.

In yet a further aspect, the present invention relates to a method of labelling a biospecific binding reactant with a compound of formula (I) or (II) as defined above comprising the steps of

a) providing a biospecific binding reactant; and
b) conjugating the biospecific binding reactant with the compound of formula (I) or (II).

In yet a further aspect, the present invention relates to the use of a detection agent as defined above in a specific bioaffinity based binding assay utilizing time-resolved fluorometric determination of a specific luminescence.

In yet a further aspect, the present invention relates to the use of a compound of formula (I) or (II) as defined above or a detection agent as defined above for the in vitro detection of an analyte in a sample.

In yet a further aspect, the present invention relates to the use of a compound of formula (I) or (II) as defined above or a detection agent as defined above in bio-imaging applications.

In yet a further aspect, the present invention relates to a solid support material conjugated with a compound of formula (I) or (II) as defined above or a detection agent as defined above.

### Definitions

As used herein, the term “linker group” refers to a moiety connecting two other moieties by at least two covalent bonds. Therefore, a linker group is a biradical group, in particular a “distance-making biradical”.

As used herein, the term “conjugating group” refers to a moiety connecting two other moieties by at least two covalent bonds or terminating a moiety in such a way that the conjugating group is conjugated with the moietie(s), preferably by way of π-electron conjugation. The moieties of the conjugating group are preferably in an arrangement so as to be conjugated with each other in order to further increase π-electron conjugation. If the conjugating group connects to other moieties, it comprises biradical moieties only, so as to form a conjugating “distance-making biradical”. However, if the conjugating group is present in a terminal position, it will in addition to the conjugating “distance-making biradical” further comprise a terminal group, which may, e.g., be selected from —H, -halogen, —CN, CH₃, or the like.

As used herein, the term “distance-making biradical” refers to a moiety that forms bonds to two other moieties with the purpose to separate the two other groups from each other, e.g. as a linker between the two other groups, e.g. to facilitate positioning a reactive group in a position accessible for reaction with a biospecific binding reactant. A distance-making biradical may comprise one or more biradical moieties. Preferred biradical moieties according to the invention include one or more, preferably 1 to 10 of the following moieties: an alkylene chain —(CH₂)₁₋₈—, ethenylene (—CH═CH—), ethynediyl (—C≡C—), an ether (—O—), a thioether (—S—), a disulfide (—S—S—), an amine (—NH— or —NR₁—), an amide (—C(═O)NH—, —C(═O)N(C₁-C₆-alkyl)-, —NHC(═O)— or —N(C₁-C₆-alkyl)C(═O)—), a ketone (—C(═O)—), a thiourea (—NH—C(═S)—NH—) and biradicals of (hetero)aromatic monocyclic or bicyclic rings (-Het/Ar—) such as phenylene, pyridylene, and triazolene. Preferred biradical moieties in conjugating groups include one or more, preferably 1 to 10 of the following moieties: ethenylene (—CH═CH—), ethynediyl (—C≡C—), carbonyl (—C(═O)—), and biradicals of (hetero)aromatic monocyclic or bicyclic rings (-Het/Ar—), e.g. phenylene, biphenylene, naphthylene, pyridylene, pyrazinylene, pyrimidinylene, pyridazinylene, furylene, thienylene, pyrrolylene, imidazolylene, pyrazolylene, thiazolylene, isothiazolylene, oxazolylene, isoxazolylene, fyrazanylene, 1,2,4-triazol-3,5-ylene, and oxadiazolylene. Further details regarding suitable biradical moieties for the present invention are provided below.

As used herein, the term “reactive group” refers to a functional group that may react in a labelling reaction of a compound of the invention with a biospecific binding reactant, or is facilitating the formation of a covalent bond to a solid support material. In case the chelate has a polymerizing group as reactive group, then the chelate may be introduced in the solid support, e.g. a particle, simultaneously with the preparation of the particles. Upon reaction with a biospecific binding reactant, the reactive group establishes a link to said biospecific binding reactant. Preferred reactive groups Z inter alia include azido (—N₃), alkynyl (—C≡CH), alkylene (—CH═CH₂), amino (—NH₂), aminooxy (—O—NH₂), carboxyl (—C(═O)OH), aldehyde (—CH(═O)), mercapto (—SH), maleimido groups or activated derivatives thereof, including isocyanato (—NCO), isothiocyanato (—NCS), diazonium (—N⁺N), bromoacetamido, iodoacetamido, reactive esters, pyridyl-2-dithio, and 6-substituted 4-chloro-1,3,5-triazin-2-ylamino and 4-chloro-1,3,5-triazin-2-yloxy. Further details in this regard are provided below.

It follows that upon reaction with a biospecific binding reactant (see further below), the reactive group Z establishes a link to said biospecific binding reactant, e.g. of one of the following types: a thiourea (—NH—C(═S)—NH—), an aminoacetamide (—NH—CO—CH₂—NH—), an amide (—NH—CO—, —CO—NH—, —NCH₃—CO— and —CO—NCH₃—), and aliphatic thioether (—S—), a disulfide (—S—S—), a 6-substituted-1,3,5-triazine-2,4-diamine, a

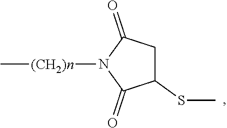

wherein n=1-6; and a triazole (e.g. formed by the so-called “click” chemistry).

As used herein, the term “hydrophilic group” refers to a moiety that is present in order to improve the water solubility of the chelate. Thus, a compound comprising a hydrophilic group as a substituent has a higher solubility in water than the corresponding compound not comprising said hydrophilic group. Examples of hydrophilic groups are provided further below and inter alia include mono- and oligosaccharides, such as monosaccharides and disaccharides, oligoalkylene glycols (e.g. those having 1-20 repeating units) such as oligoethylene glycol and oligopropylene glycol, and the like.

As used herein, the term “monosaccharide is intended to mean C₅-C₇ carbohydrates being either in the acyclic or in cyclic form. Preferred examples are provided further below.

As used herein, the term “oligosaccharide” refers to a saccharide polymer containing a small number, typically from 3 to 10 units of monosaccharides mentioned above, which are preferably linked together by glycosidic bonds.

As used herein the term “polysaccharide” refers to a saccharide polymer containing more than 10 units of monosaccharides, preferably linked together by glycosidic bonds.

As used herein, the term “chelate” or “chelate moiety” is a chemical structure or compound composed of a metal ion and a chelating ligand, which contains chelating groups. Chelating ligand refers to a moiety that inter alia coordinates with several bonds (i.e. coordination bonds) of the chelating groups to the metal ion of the chelate, and forms from five to six membered rings with the metal ion. Examples of chelating groups thus include, but are not limited to, groups comprising at least one of primary, secondary or tertiary amine, —C(═O)—, —C(═O)O⁻, —C(═O)NH—, —P(═O)O₂²⁻, —P(═O)MeO⁻, —P(═O)PhO⁻ group, wherein the nitrogen or oxygen forms a coordination coordinate bond with the metal ion of the chelate. Preferred metal ions according to the present invention are lanthanide ions Ln³⁺. The molecules of the present invention contain from two to three separate internal chelating moieties, which are preferably selected from the following chelating moieties:

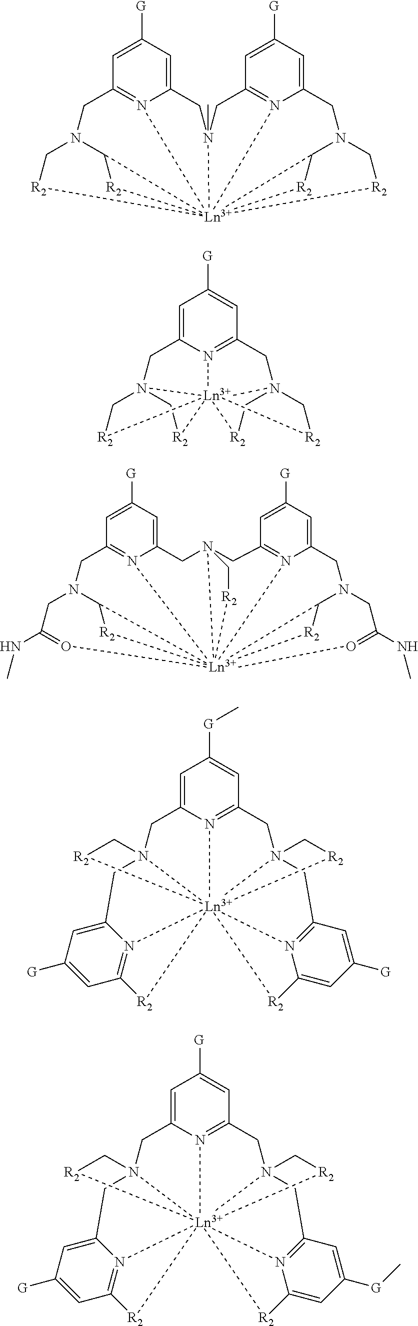

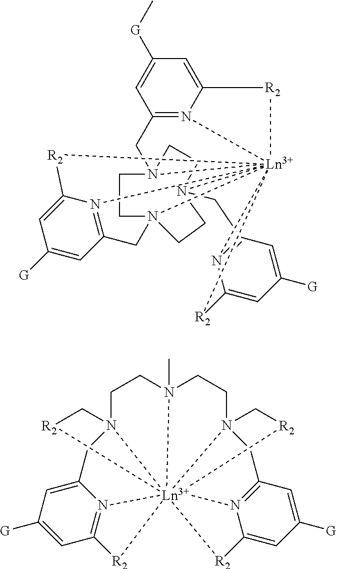

In these structures, the dashed lines between the chelating groups and the Ln³⁺ ion represent the coordination bonds of the chelating moiety in question.

As used herein, the term “lanthanide ion” or “LLn³⁺” is intended to mean a trivalent ion of the lanthanide series of the Periodic Table of Elements, e.g. europium(III), terbium(III), samarium(III) and dysprosium(III), i.e. EuLn³⁺, TbLn³⁺, SmLn³⁺ or DyLn³⁺. In many embodiments, europium(III) (EuLn³⁺) and terbium(III) (TbLn³⁺) are preferred. EuLn³⁺ is particularly preferred.

It should be understood that in some embodiments the basic structure of the lanthanide chelate of the formula (I) (as well as the lanthanide chelating ligand of the formula (II)) may comprise at least two negative charges, and even more negative charges depending on the substituents in formula (I) or (II). If the chelate or the ligand comprises a negative charge, according to common knowledge in the field, they may be associated with counter ions to form salts. Hence, it should be understood that the compounds, respectively, in addition to what is illustrated in formula (I) and formula (II), may be further associated with one or more cations as counter ions to form “salts”. Examples of such counter ions are Na⁺, CaLn²⁺, and K⁺. Particularly preferred are Na⁺ and K⁺. Preferably, the counter ions are those from Groups IA and IIA of the periodic table of elements. The metal ion Ln³⁺, which is bound by coordinate bonds in the chelate, is not considered as a counter ion in a salt. In other embodiments, the lanthanide chelate as well as the lanthanide chelating ligand or chelating ligand may have a neutral net charge, wherein the term “net charge” refers to the sum of the positive and negative charges of a molecule comprising a ligand and lanthanide ion(s). The net charge of the molecule is of course dependent of the chosen chelating groups and used substituents or groups in the conjugating group or for example linker group. For example, if the groups Che₁ and Che₂ are chosen to be of formula Che V, chelating groups R₂ are —COO⁻ and the substituents in the conjugating groups are chosen suitably, the net charge of the molecule can be neutral, and therefore the molecule does not comprise at least two negative charges.

As used herein, the term “biospecific binding reactant” is a compound capable of specifically binding an analyte of interest for the purpose of quantitative or qualitative analysis of said analyte in a sample (e.g. a sample of a bodily fluid).

As used herein, the term “antibody” refers to the commonly known Y-shaped protein produced mainly by plasma cells that is used by the immune system to neutralize pathogens such as pathogenic bacteria and viruses. However, the term “antibody” as used in the context of the present invention also comprises molecules derived from such antibodies, such as a Fab-fragment, Fab2, FC-fragment, diabodies and the like.

As used herein, an “antigen” is a molecule capable of inducing an immune response to produce an antibody. Thus, an antigen may be a molecule binding to an antibody.

As used herein, a “receptor ligand” is a molecule that is known to bind to a cell receptor. Examples of receptor ligands are neurotransmitters, hormones, growth factors or the like. Examples of corresponding receptors are G-protein coupled receptors, protein kinase receptors and the like.

As used herein, the term “DNA probe” or “RNA probe” refers to a ribonucleic acid or a deoxynucleic acid that may hybridize as a “probe” with a target nucleic acid sequence. Thus, the probe may comprise a complementary sequence to a target nucleic acid sequence.

As used herein, the term “protein” covers any type of protein, including enzymes or specific binding proteins that interact specifically with one or more target molecules. In the sense of the present invention, the term protein refers to any polymer of amino acids of any length, and therefore also covers peptides e.g. oligopeptides which only comprise 2 to 100 amino acids.

As used herein, the term “phospholipid” refers to a class of lipids that are a major component of all cell membranes. The structure of the phospholipid molecule generally consists of two hydrophobic fatty acid “tails” and a hydrophilic “head” consisting of a phosphate group. The two components are joined together by a glycerol molecule. The phosphate groups can be modified with simple organic molecules such as choline.

As used herein, the term “PNA” or “peptide nucleic acid” refers to an artificially synthesized polymer similar to RNA or DNA. However, DNA and RNA have a deoxyribose and ribose sugar backbone, respectively, whereas PNA's backbone is composed of repeating —N-(2-aminoethyl)-glycine units linked by peptide bonds. The various purine and pyrimidine bases are linked to the backbone by a methylene bridge (—CH₂—) and a —(C═O)— group.

As used herein, the term “steroid” refers to an organic compound with four rings arranged in a specific molecular configuration. Examples include the dietary lipid cholesterol, the sex hormones estradiol and testosterone and the anti-inflammatory drug dexamethasone. Steroids have two principal biological functions: certain steroids (such as cholesterol) are important components of cell membranes which alter membrane fluidity, and many steroids are signaling molecules which activate steroid hormone receptors.

The steroid core structure is composed of seventeen carbon atoms, bonded in four “fused” rings: three six-membered cyclohexane rings and one five-membered cyclopentane ring. Steroids vary by the functional groups attached to this four-ring core and by the oxidation state of the rings. Sterols are forms of steroids with a hydroxyl group at position three and a skeleton derived from cholestane. They can also vary more markedly by changes to the ring structure as for example in ring scissions which produce secosteroids such as vitamin D3.

As used herein, the term “hapten” refers to a molecule that elicits an immune response only when attached to a large carrier molecule such as a protein, wherein the carrier may be one that preferably does not elicit an immune response by itself.

As used herein, the term “drug” refers to a compound with pharmacological properties that may be used in the preventive or therapeutic treatment of a disease.

As used herein, the term “lectin” refers to carbohydrate-binding proteins, i.e. macromolecules that are highly specific for sugar moieties.

As used herein, the term “oligonucleotide” refers to short DNA or RNA molecules (comprising less than 30 monomers), oligomers, that have a wide range of applications in genetic testing, research, and forensics. Commonly made in the laboratory by solid-phase chemical synthesis, these small bits of nucleic acids can be manufactured as single-stranded molecules with any user-specified sequence, and so are vital for artificial gene synthesis, polymerase chain reaction (PCR), DNA sequencing, library construction and as molecular probes. In nature, oligonucleotides are usually found as small RNA molecules that function in the regulation of gene expression (e.g. microRNA), or are degradation intermediates derived from the breakdown of larger nucleic acid molecules.

Oligonucleotides are characterized by the sequence of nucleotide residues that make up the entire molecule.

“Modified oligonucleotides” refer to oligonucleotides that comprise one or more non-natural nucleic acid, i.e. a nucleic acid that does not comprise cytosine, guanine, adenine, thymine or uracil as base.

A “polynucleotide” refers to a biopolymer composed of 30 or more nucleotide monomers covalently bonded in a chain.

A “modified polynucleotide” refers to polynucleotide that comprises one or more non-natural nucleic acid monomers.

As used herein, the term “analyte” refers to a target parameter of interest to be determined qualitatively and/or quantitatively in a sample.

As used herein, the term “biospecific binding assay” or “specific bioaffinity based binding assay” means an in vitro assay wherein a specific complex is formed between a biomolecule and a target molecule and the presence of the complex, i.e. the binding, may be detectable by standard biochemical methods.

As used herein, the term “biospecific binding reactant” means a biomolecule that may specifically bind to an analyte of interest under conditions present in a biospecific binding assay.

As used herein, the term “detection agent” means a compound that is detectable e.g. in an in vitro assay format, e.g. by luminescence or UV-VIS absorbance.

As used herein, the term “specific luminescence” refers to the luminescence of a detection agent, wherein the luminescence is measured in a way that ensures that the luminescence is specifically related to the detection agent, e.g. by selecting a wavelength for light measurement where the detection agent shows a high emission of light, e.g. close to the emission maximum in the spectra of the detection agent.

As used herein, the term “luminescence” refers to the emission of light by a substance not resulting from heat. Examples of luminescence are chemiluminescence, bioluminescence, photoluminescence, phosphorescence and the like.

As used herein, the term “sample” refers to a sample collected from a patient for use in in vitro bioassays to determine an analyte parameter of interest, wherein the sample is typically a bodily fluid, such as blood, saliva, urine, cerebrospinal fluid, or a tissue sample such as a biopsy sample.

As used herein, the term “activated derivative” of azido (—N₃), —C≡CH, —CH═CH₂, amino (—NH₂), aminooxy (—O—NH₂), carboxyl (—COOH), aldehyde (—CHO), mercapto (—SH), and maleimido groups refers to activated forms of these functional groups, which are reactive, so that labelling of biospecific binding reactants is possible. The activated derivatives include, but are not limited to, isocyanato (—NCO), isothiocyanato (—NCS), diazonium (—N⁺N), bromoacetamido, iodoacetamido, reactive esters, pyridyl-2-dithio, 6-substituted 4-chloro-1,3,5-triazin-2-ylamino and 6-substituted 4-chloro-1,3,5-triazin-2-yloxy, wherein the 6-substituted 4-chloro-1,3,5-triazin-2-ylamino is preferably 4,6-dichloro-1,3,5-triazin-2-ylamino and the 6-substituted 4-chloro-1,3,5-triazin-2-yloxy is preferably 4-chloro-1,3,5-triazin-2-yloxy.

As used herein, the term “reactive ester” refers to esters, which are activated, e.g., for amide bond formation and peptide coupling, and have a higher reactivity than alkyl or benzyl esters. Suitable reactive esters are described in the article by Christian A. G. N. Montalbetti and V. Falque in Tetrahedron 61 (2005) 10827 and preferably include aromatic esters based on p-nitrophenol, pentafluorophenol, 2,4,5-trichlorophenol, N-hydroxy-5-norbornene-endo-2,3-dicarboxyimide, hydroxybenzotriazole, 1-hydroxy-7-azabenzoptriazole, sulfo-N-hydroxysuccinimide, or N-hydroxysuccinimide, esters based on phosphonium-, uronium-, or guanidinium-based coupling reagents, and triazinyl or pyridinium esters.

The organic moieties mentioned in the above definitions of the variables are—like the term halogen—collective terms for individual listings of the individual group members. The prefix Cₙ-Cₘ indicates in each case the possible number of carbon atoms in the group.

The term “halogen” denotes in each case fluorine, bromine, chlorine or iodine, in particular fluorine, chlorine and bromine.

The term “alkyl” as used herein denotes in each case a straight-chain or branched, non-cyclic saturated hydrocarbon having usually from 1 to 12 carbon atoms, frequently from 1 to 6 carbon atoms, preferably from 1 to 4 carbon atoms, more preferably from 1 to 3 carbon atoms. Representative straight chain —C₁₋₁₂ alkyl groups include methyl, -ethyl, -n-propyl, -n-butyl, -n-pentyl, -n-hexyl, n-heptyl, n-octyl, n-nonyl and n-decyl. Representative branched —(C₁-C₁₂)alkyl groups include isopropyl, sec-butyl, isobutyl, tert-butyl, isopentyl, neopentyl, 1-methylbutyl, 2-methylbutyl, 3-methylbutyl, 1,1-dimethylpropyl, 1,2-dimethylpropyl, 1-methylpentyl, 2-methylpentyl, 3-methylpentyl, 4-methylpentyl, 1-ethylbutyl, 2-ethylbutyl, 3-ethylbutyl, 1,1-dimethylbutyl, 1,2-dimethylbutyl, 1,3-dimethylbutyl, 2,2-dimethylbutyl, 2,3-dimethylbutyl, 3,3-dimethylbutyl, 5-methylhexyl, 6-methylheptyl, and the like.

The term “haloalkyl” as used herein denotes in each case a straight-chain or branched alkyl group having usually from 1 to 12 carbon atoms, frequently from 1 to 6 carbon atoms, preferably from 1 to 4 carbon atoms, wherein the hydrogen atoms of this group are partially or totally replaced with halogen atoms. Preferred haloalkyl moieties are selected from C₁-C₄-haloalkyl, more preferably from C₁-C₃-haloalkyl or C₁-C₂-haloalkyl, in particular from C₁-C₂-fluoroalkyl such as fluoromethyl, difluoromethyl, trifluoromethyl, 1-fluoroethyl, 2-fluoroethyl, 2,2-difluoroethyl, 2,2,2-trifluoroethyl, pentafluoroethyl, and the like.

The term “5- to 10-membered aromatic or heteroaromatic monocyclic or bicyclic ring” (wherein the aromatic monocyclic or bicyclic rings may also be referred to as aryl or —Ar, and the heteroaromatic monocyclic or bicyclic rings may also be referred to as hetaryl or heteroaryl or —Het) as used herein denotes an aromatic or heteroaromatic monocyclic or bicyclic ring having from 5 to 10 atoms as ring members. Preferred heteroaromatic monocyclic rings are 5- or 6-membered rings. Preferred heteroaromatic bicyclic rings are 9- or 10-membered rings. Preferred aromatic monocyclic rings are 6-membered rings. Preferred aromatic bicyclic rings are 9- or 10-membered rings. For bicyclic aromatic or heteroaromatic rings it is only required that one of the two rings is aromatic. Alternatively, both rings are aromatic. While the aromatic rings comprise only carbon atoms as ring members, in heteroaromatic rings at least one carbon atom (of one or both rings) is replaced with one or more, same or different heteroatoms selected from nitrogen (N), oxygen (O), and sulfur (S). It is to be understood that the sulfur atom may optionally be present in oxidized form. In one embodiment, one of the bicyclic -(5- to 10-membered)heteroaryl rings contains at least one carbon atom. In another embodiment, both of the bicyclic -(5- to 10-membered)heteroaryl rings contain at least one carbon atom. Representative -(5- to 10-membered)heteroaryls include pyridyl, furyl, benzofuranyl, thiophenyl, benzothiophenyl, quinolinyl, isoquinolinyl, pyrrolyl, indolyl, oxazolyl, benzoxazolyl, imidazolyl, benzimidazolyl, thiazolyl, benzothiazolyl, isoxazolyl, oxadiazolinyl, pyrazolyl, isothiazolyl, pyridazinyl, pyrimidyl, pyrimidinyl, pyrazinyl, thiadiazolyl, triazinyl, thienyl, thiadiazolyl, cinnolinyl, phthalazinyl, quinazolinyl, and the like. Representative —(6- to 10-membered)aryl groups include indenyl, -phenyl, and -naphthyl and the like. Phenyl is particularly preferred.

### FIGURES

FIG. 1 shows ligand esters 24 and 25, which are preferred compounds of formula (II) as described herein.

FIG. 2 shows Eu chelates 26, 27, 28, and 29, which are preferred compounds of formula (I) as described herein.

FIG. 3 shows ligand ester 34, which is a preferred compound of formula (II) as described herein.

FIG. 4 shows Eu chelates 35 and 36, which are preferred compounds of formula (I) as described herein.

FIG. 5 shows ligand esters 43 and 44, which are preferred compounds of formula (II) as described herein.

FIG. 6 shows Eu chelates 45, 46, 47, and 48, which are preferred compounds of formula (I) as described herein.

FIG. 7 shows ligand ester 55, which is a preferred compound of formula (II) as described herein.

FIG. 8 shows Eu chelates 56 and 57, which are preferred compounds of formula (I) as described herein.

FIG. 9 shows Eu chelates 58 and 59, which are preferred compounds of formula (I) as described herein.

FIG. 10 shows reference Eu chelates Ref 1, Ref 2 and Ref 3, which comprise only one chelate moiety.

### DETAILED DESCRIPTION

As indicated above, the present invention relates to a compound of formula (I)

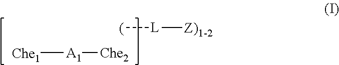

or a salt thereof, wherein
(i) the solid lines represent covalent bonds;
(ii) the dashed line represents a covalent bond of the group -L-Z to any one of the groups Che₁, A₁, and Che₂;
   and wherein
L is in each case independently absent or selected from linker groups comprising from 1 to 10 moieties selected from —(CH₂)₁₋₈ —, —CH═CH—, —C≡C—, —O—, —S—, —S—S—, —C(═O)—, —C(═O)NH—, —NHC(═O)—, —C(═O)N(C₁-C₆-alkyl)-, —N(C₁-C₆-alkyl)C(═O)—, —NHC(═S)NH—, —CH[(CH₂)₀₋₆C(═O)O⁻]—, —CH[(CH₂)₀₋₆C(═O)OH]—, and biradicals of 5- to 10-membered aromatic or heteroaromatic monocyclic or bicyclic rings, wherein the heteroaromatic ring contains one or more, same or different heteroatoms N, O, or S;
Z is in each case independently selected from reactive groups selected from —N₃, —C≡CH, —CH═CH₂, —NH₂, —C(═O)OH, —CH(═O), —SH, —OH, maleimido and activated derivatives thereof including —NCO, —NCS, —N⁺≡N, bromoacetamido, iodoacetamido, reactive esters, pyridyl-2-dithio, and 6-substituted 4-chloro-1,3,5-triazin-2-ylamino and 4-chloro-1,3,5-triazin-2-yloxy; wherein the substituent in the 6-position of the 4-chloro-1,3,5-triazin-2-ylamino or 4-chloro-1,3,5-triazin-2-yloxy is selected from —H, -halogen, —SH, —NH₂, —C₁-C₆-alkyl, —O(C₁-C₆-alkyl), —OAryl, —S(C₁-C₆-alkyl), —SAryl, —N(C₁-C₆-alkyl)₂, and N(Aryl)₂;
   wherein the carbon atoms of the aforementioned groups are unsubstituted or substituted by one or more substituents selected from —CN, -halogen, —SH, —C(═O)H, —C(═O)OH, C₁-C₆-alkyl, C₁-C₆-haloalkyl, —O(C₁-C₆-alkyl), —C(═O)(C₁-C₆-alkyl), —C(═O)O(C₁-C₆-alkyl), and phenyl;
A₁ is a bridging group comprising from one to three separate straight or branched, saturated or unsaturated carbon-based chains including from 1 to 12 carbon atoms, wherein the carbon-based chains are free of or comprise one to ten, same or different groups selected from —O—, —S—, —NH—, —NR₁—, —C(═O)NH—, —NHC(═O)—, —C(═O)NR₁—, —NR₁C(═O)— and —C(═O)—,
   or
A₁ a bridging chelate moiety -Che₃- of the following general formula

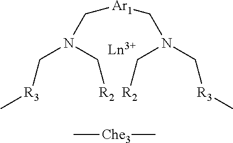

and wherein
Che₁ and Che₂ are independently selected from the chelate moieties Che I, Che II, Che III, Che IV, Che V, Che VI, and Che VII of the following general formulae:

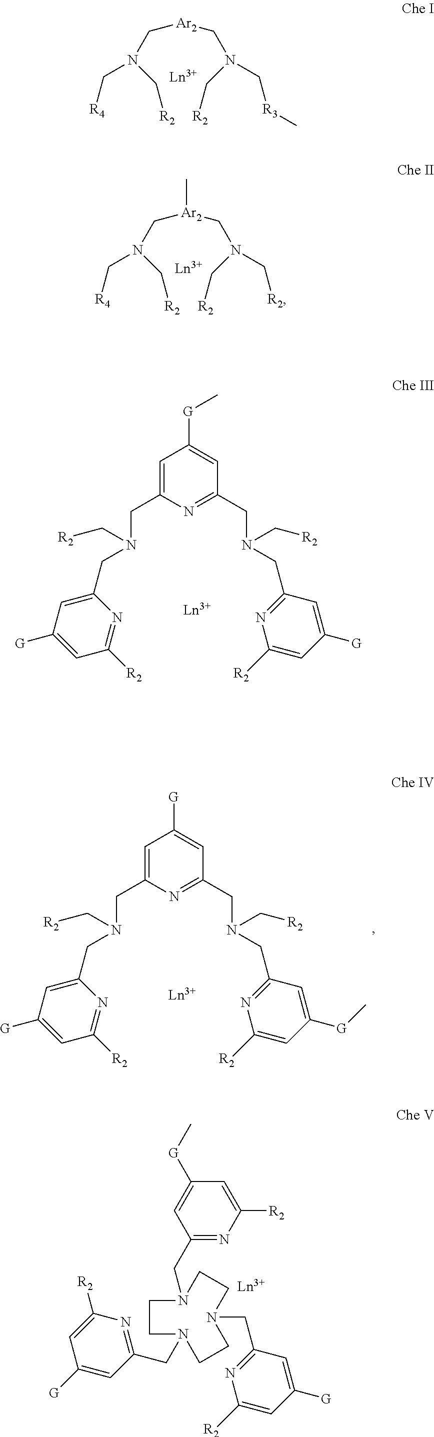

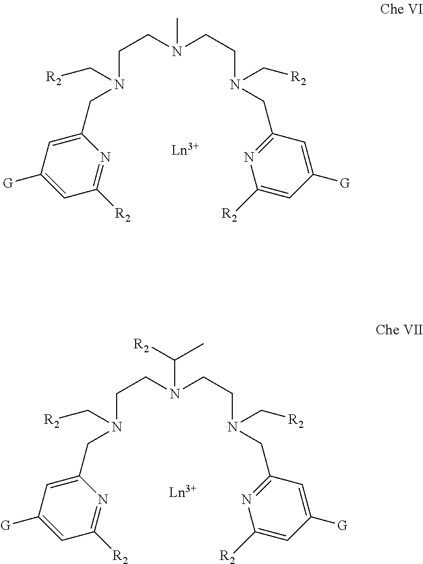

and wherein
R₁ is in each case independently selected from C₁-C₆-alkyl, and from the option of representing one of the one or two groups -L-Z;
R₂ is in each case independently selected from —C(═O)O⁻, —P(═O)O₂²⁻, P(═O)MeO⁻, —P(═O)PhO⁻, and the C₁-C₆-alkyl esters thereof, and from the option of representing one of the one or two groups -L-Z;
R₃ is in each case independently selected from the bridging groups —C(═O)NH—, —C(═O)NH—(C₁-C₆-alkylene)-NHC(═O)—, —C(═O)NH—(C₁-C₆-alkylene)-O—, —C(═O)NH—(C₁-C₆-alkylene)-C(═O)NH—, —C(═O)NH—(C₁-C₆-alkylene)-S—, —C(═O)NH—(C₁-C₆-alkylene)-NHC(═O)—(C₁-C₆-alkylene)-NHC(═O)—, —C(═O)NH—(C₁-C₆-alkylene)-NHC(═O)—(C₁-C₆-alkylene)-O—, —C(═O)NH—(C₁-C₆-alkylene)-NHC(═O)—(C₁-C₆-alkylene)-C(═O)NH—, —C(═O)NH—(C₁-C₆-alkylene)-NHC(═O)—(C₁-C₆-alkylene)-S—, —C(═O)NH—(C₁-C₆-alkylene)-O—(C₁-C₆-alkylene)-NHC(═O)—, —C(═O)NH—(C₁-C₆-alkylene)-O—(C₁-C₆-alkylene)-O—, —C(═O)NH—(C₁-C₆-alkylene)-O—(C₁-C₆-alkylene)-S—, —C(═O)NH—(C₁-C₆-alkylene)-O—(C₁-C₆-alkylene)-C(═O)NH—, —C(═O)NH—(C₁-C₆-alkylene)-C(═O)NH—(C₁-C₆-alkylene)-NHC(═O)—, —C(═O)NH—(C₁-C₆-alkylene)-C(═O)NH—(C₁-C₆-alkylene)-O—, —C(═O)NH—(C₁-C₆-alkylene)-C(═O)NH—(C₁-C₆-alkylene)-S—, —C(═O)NH—(C₁-C₆-alkylene)-C(═O)NH—(C₁-C₆-alkylene)-C(═O)NH—, and —P(═O)O₂²⁻;
R₄ is in each case independently selected from —CH₂N(CH₂C(═O)O⁻)₂, —CH₂N(CH₂P(═O)O₂²⁻)₂, —CH₂N(CH₂P(═O)MeO)₂, —CH₂N(CH₂P(═O)PhO⁻)₂, and from the options defined for R₂;
Ln³⁺ is in each case independently selected from the lanthanide ions Eu³⁺, Tb³⁺, Sm³⁺ and Dy³⁺, wherein the lanthanide ion forms from seven to ten coordination bonds with the heteroatoms oxygen and nitrogen in the chelate moieties Che₁, Che₂, and Che₃ to form from two to three separate internal chelate moieties;
Ar₁ is selected from the following groups

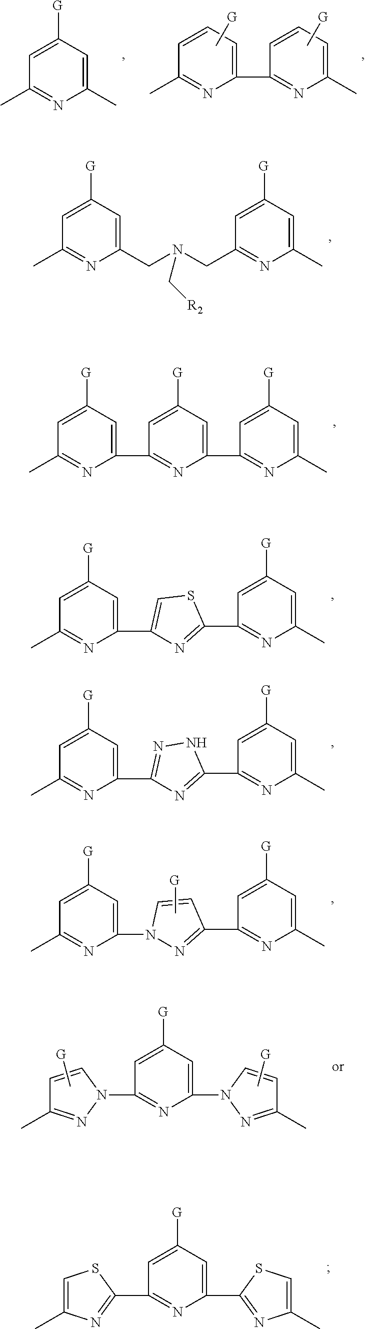

Ar₂ is in each case independently selected from the following groups

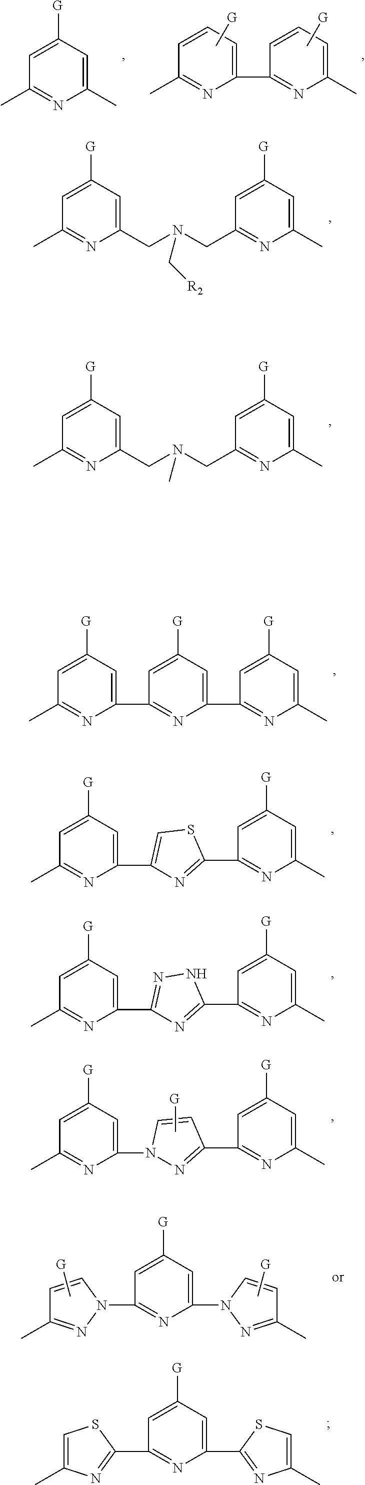

and wherein
G is in each case independently selected from i) a conjugating group, ii) a single bond, and iii) hydrogen;
   wherein each conjugating group comprises 1, 2, or 3 moieties selected from —CH═CH—, —C≡C—, —C(═O)—, and biradicals of 5 to 10-membered aromatic or heteroaromatic monocyclic or bicyclic rings, wherein the heteroaromatic ring contains one or more, same or different heteroatoms N, O, or S, and wherein the aromatic or heteroaromatic monocyclic or bicyclic rings are unsubstituted or substituted by 1 to 5 same or different substituents R₅;
   wherein each conjugating group, if present in a terminal position, may further comprise a terminal group, which is selected from the group consisting of —H, -halogen, —CN, —CH₃, and from the option of representing one of the one or two groups -L-Z.
   wherein R₅ is independently selected from C₁-C₁₂-alkyl, —(CH₂)₀₋₆—C(═O)OH, —(CH₂)₀₋₆—C(═O)O⁻, —(CH₂)₀₋₆—S(═O)₂OH, —(CH₂)₀₋₆—S(═O)₂O⁻, —C(═O)NHR₆, —C(═O)NCH₃R₆, —NHC(═O)NHR₆, —NHC(═S)NHR₆, -halogen, —OH, —SH, —OR₇, —SR₇, and hydrophilic groups selected from monosaccharides, disaccharides, —(CH₂)₁₋₆ CH₂OH, —CH(CH₂OH)₂, —C(CH₂OH)₃, —(CH₂)₁₋₃—O—(CH₂CH₂O)₀₋₅—H, —(CH₂)₁₋₃—O—(CH₂CH₂O)₀₋₅—C₁-C₄-alkyl, —O—(CH₂CH₂O)₁₋₆—H, and —O—(CH₂CH₂O)₁₋₆—C₁-C₄-alkyl;
   wherein R₆ is selected from C₁-C₁₂-alkyl, —(CH₂)₁₋₆C(═O)OH, —(CH₂)₁₋₆C(═O)O⁻, —(CH₂)₁₋₆ S(═O)₂OH, —(CH₂)₁₋₆ S(═O)₂O⁻ and hydrophilic groups selected from monosaccharides, disaccharides, —(CH₂)₁₋₆CH₂OH, —CH(CH₂OH)₂, —C(CH₂OH)₃, —(CH₂)₁₋₃—O—(CH₂CH₂O)₀₋₅—H, —(CH₂)₁₋₃—O—(CH₂CH₂O)₀₋₅—C₁-C₄-alkyl, —O—(CH₂CH₂O)₁₋₆—H, and —O—(CH₂CH₂O)₁₋₆—C₁-C₄-alkyl;
   wherein R₇ is selected from —CF₃, —C₁-C₁₂-alkyl, —(CH₂)₁₋₆C(═O)OH, —(CH₂)₁₋₆C(═O)O⁻, —(CH₂)₁₋₆ S(═O)₂OH, —(CH₂)₁₋₆ S(═O)₂O⁻, —C(═O)NHR₆, —C(═O)NCH₃R₆, —NHC(═O)NHR₆, —NHC(—S)NHR₆, —(CH₂)₁₋₆ N(CH₃)₂⁺—(CH₂)₁₋₆ S(═O)₂O⁻, —(CH₂)₁₋₆C(═O)-(piparazin-1,4-diyl)-(CH₂)₁₋₆C(═O)OH, and hydrophilic groups selected from monosaccharides, disaccharides, —(CH₂)₁₋₆CH₂OH, —CH(CH₂OH)₂, —C(CH₂OH)₃, —(CH₂)₁₋₃—O—(CH₂CH₂O)₀₋₅—H, —(CH₂)₁₋₃—O—(CH₂CH₂O)₀₋₅—C₁-C₄-alkyl, —O—(CH₂CH₂O)₁₋₆—H, and —O—(CH₂CH₂O)₁₋₆—C₁-C₄-alkyl.

Preferred embodiments of the compounds of formula (I) are defined hereinafter. It is to be understood that the preferred embodiments regarding the groups and substituents of the compounds of formula (I) are also preferred in connection with the compounds of formula (II), as well as in connection with the detection agent of the invention, the methods and uses of the invention, and the solid support material of the invention.

The compounds of formula (I) comprise from two to three separate lanthanide chelate moieties, which are covalently tethered to each other. In particular, Che₁ and Che₂ are independently selected from the chelate moieties Che I, Che II, Che III, Che IV, Che V, Che VI, and Che VII as defined above. In one embodiment of the invention, A₁ represents a bridging group comprising from one to three separate straight or branched, saturated or unsaturated carbon-based chains including from 1 to 12 carbon atoms, wherein the carbon-based chains are free of or comprise one to ten, same or different groups selected from —O—, —S—, —NH—, —NR₁—, —C(═O)NH—, —NHC(═O)—, —C(═O)NR₁—, —NR₁C(═O)— and —C(═O)—. This results in compounds of formula (I) comprising only the two lanthanide chelate moieties Che₁ and Che₂. In another embodiment of the invention, A₁ represents a bridging chelate moiety -Che₃- as defined above. This results in compounds of formula (I) comprising three lanthanide chelate moieties, i.e. Che₁, Che₂, and Che₃.

The lanthanide chelate moieties preferably comprise from one to three individual chromophore moieties around an emitting lanthanide ion. Preferably, the lanthanide ion in each case forms from seven to ten coordination bonds with the heteroatoms oxygen and nitrogen in the chelate moieties Che₁, Che₂, and Che₃.

In one embodiment, the lanthanide ion Ln³⁺ is in each case independently selected from the lanthanide ions Eu³⁺, Tb³⁺, Sm³⁺ and Dy³⁺, wherein the lanthanide ion forms from seven to ten coordination bonds with the heteroatoms oxygen and nitrogen in the chelate moieties Che₁, Che₂, and Che₃. In a preferred embodiment, the lanthanide ion Ln³⁺ is in each case Eu³⁺, which forms from seven to ten coordination bonds with the heteroatoms oxygen and nitrogen in the chelate moieties Che₁, Che₂, and Che₃.

The compounds of formula (I) further preferably comprise one or more conjugating groups as substituents G, wherein each conjugating group comprises 1, 2, or 3 moieties selected from —CH═CH—, —C≡C—, —C(═O)—, and biradicals of 5 to 10-membered aromatic or heteroaromatic monocyclic or bicyclic rings, wherein the heteroaromatic ring contains one or more, same or different heteroatoms N, O, or S, and wherein the aromatic or heteroaromatic monocyclic or bicyclic rings are unsubstituted or substituted by 1 to 5 same or different substituents R₅, wherein R₅ is defined as above; and wherein each conjugating group, if present in a terminal position, may further comprise a terminal group, which is selected from the group consisting of —H, -halogen, —CN, —CH₃, and from the option of representing one of the one or two groups -L-Z. In a preferred embodiment, each conjugating group comprises 1, 2, or 3 moieties selected from —CH═CH—, —C≡C—, —C(═O)—, phenylene, biphenylene, naphthylene, pyridylene, pyrazinylene, pyrimidinylene, pyridazinylene, furylene, thienylene, pyrrolylene, imidazolylene, pyrazolylene, thiazolylene, isothiazolylene, oxazolylene, isoxazolylene, fyrazanylene, 1,2,4-triazol-3,5-ylene, and oxadiazolylene, wherein the aromatic or heteroaromatic monocyclic or bicyclic rings are unsubstituted or substituted by 1 to 5 same or different substituents R₅, and more preferably each conjugating group is independently selected from phenylene-C≡C—, phenylene, thienylene, and furylene.

Typically, the conjugating groups are attached to the pyridine groups of the chelate moieties. This may enhance the luminescence intensity by increasing the chromophore's molar absorptivity due to an increased π-electron conjugation of the aromatic chromophore. Therefore, in a preferred embodiment, each conjugating group comprises the 1, 2 or 3 moieties in an arrangement so as to be conjugated with each other and attached to the respective pyridine in such a way that the conjugating group is conjugated with the pyridine.

The compounds of formula (I) further comprise one or two reactive groups Z in order to allow a covalent attachment to a biospecific binding reactant. The reactive group Z is connected to any one of the groups Che₁, A₁, and Che₂ of the compounds of formula (I) via a group L, which is a linker group, also referred to as spacer, i.e. a distance-making biradical, so as—if necessary or desirable—to position the reactive group Z in a position accessible for reaction with the biospecific binding reactant.

In one embodiment of the invention,
L is in each case independently absent or selected from linker groups comprising from 1 to 10 moieties selected from —(CH₂)₁₋₈ —, —CH═CH—, —C≡C—, —O—, —S—, —S—S—, —C(═O)—, —C(═O)NH—, —NHC(═O)—, —C(═O)N(C₁-C₆-alkyl)-, —N(C₁-C₆-alkyl)C(═O)—, —NHC(═S)NH—, —CH[(CH₂)₀₋₆C(═O)O⁻]—, —CH[(CH₂)₀₋₆C(═O)OH]—, and biradicals of 5- to 10-membered aromatic or heteroaromatic monocyclic or bicyclic rings, wherein the heteroaromatic ring contains one or more, same or different heteroatoms N, O, or S.

In a preferred embodiment of the invention,
L is in each case independently absent or selected from linker groups comprising from 1 to 10 moieties selected from —(CH₂)₁₋₈ —, —CH═CH—, —C≡C—, —O—, —S—, —S—S—, —C(═O)—, —C(═O)NH—, —NHC(═O)—, —C(═O)N(C₁-C₆-alkyl)-, —N(C₁-C₆-alkyl)C(═O)—, —NHC(═S)NH—, —CH[(CH₂)₀₋₆C(═O)O⁻]—, —CH[(CH₂)₀₋₆C(═O)OH]—, phenylene, pyridylene, and triazolene.

In a more preferred embodiment of the invention,
L is in each case independently absent or selected from linker groups comprising from 1 to 10 moieties selected from —(CH₂)₁₋₈ —, —C≡C—, —O—, —C(═O)—, —C(═O)NH—, —NHC(═O)—, phenylene, pyridylene, and triazolene.

In an even more preferred embodiment of the invention,
L is in each case independently absent or selected from linker groups comprising from 1 to 10 moieties selected from —(CH₂)₁₋₈ —, —O—, —C(═O)—, —NHC(═O)—, —C(═O)NH— and phenylene.

In one embodiment of the invention,
Z is in each case independently selected from reactive groups selected from —N₃, —C≡CH, —CH═CH₂, —NH₂, —O—NH₂, —C(═O)OH, —CH(═O), —SH, —OH, maleimido and activated derivatives thereof including —NCO, —NCS, —N⁺≡N, bromoacetamido, iodoacetamido, reactive esters, pyridyl-2-dithio, and 6-substituted 4-chloro-1,3,5-triazin-2-ylamino and 4-chloro-1,3,5-triazin-2-yloxy; wherein the substituent in the 6-position of the 4-chloro-1,3,5-triazin-2-ylamino or 4-chloro-1,3,5-triazin-2-yloxy is selected from —H, -halogen, —SH, —NH₂, —C₁-C₆-alkyl, —O(C₁-C₆-alkyl), —OAryl, —S(C₁-C₆-alkyl), —SAryl, —N(C₁-C₆-alkyl)₂, and N(Aryl)₂;
   wherein the carbon atoms of the aforementioned groups are unsubstituted or substituted by one or more substituents selected from —CN, -halogen, —SH, —C(═O)H, —C(═O)OH, C₁-C₆-alkyl, C₁-C₆-haloalkyl, —O(C₁-C₆-alkyl), —C(═O)(C₁-C₆-alkyl), —C(═O)O(C₁-C₆-alkyl), and phenyl;

Suitable reactive esters are described in the article by Christian A. G. N. Montalbetti and V. Falque in Tetrahedron 61 (2005) 10827 and preferably include aromatic esters based on p-nitrophenol, pentafluorophenol, 2,4,5-trichlorophenol, N-hydroxy-5-norbornene-endo-2,3-dicarboxyimide, hydroxybenzotriazole, 1-hydroxy-7-azabenzoptriazole, sulfo-N-hydroxysuccinimide, or N-hydroxysuccinimide, esters based on phosphonium-, uronium-, or guanidinium-based coupling reagents, and triazinyl or pyridinium esters. Preferred reactive esters are selected from the following reactive esters:

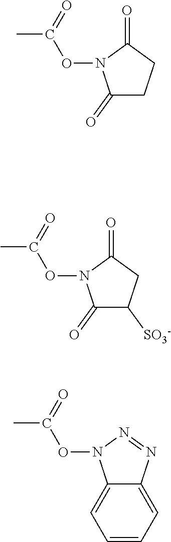

In a preferred embodiment of the invention,
Z is in each case independently selected from reactive groups selected from —N₃, —C≡CH, —CH═CH₂, —NH₂, —O—NH₂, —C(═O)OH, —CH(═O), —SH, —OH, maleimido, —NCO, —NCS, —N⁺≡N, bromoacetamido, iodoacetamido, aromatic esters based on p-nitrophenol, pentafluorophenol, 2,4,5-trichlorophenol, N-hydroxy-5-norbornene-endo-2,3-dicarboxyimide, hydroxybenzotriazole, 1-hydroxy-7-azabenzoptriazole, sulfo-N-hydroxysuccinimide, or N-hydroxysuccinimide, esters based on phosphonium-, uronium-, or guanidinium-based coupling reagents, triazinyl or pyridinium esters, pyridyl-2-dithio, and 6-substituted 4-chloro-1,3,5-triazin-2-ylamino and 4-chloro-1,3,5-triazin-2-yloxy; wherein the substituent in the 6-position of the 4-chloro-1,3,5-triazin-2-ylamino or 4-chloro-1,3,5-triazin-2-yloxy is selected from —H, -halogen, —SH, —NH₂, —C₁-C₆-alkyl, —O(C₁-C₆-alkyl), —OAryl, —S(C₁-C₆-alkyl), —SAryl, —N(C₁-C₆-alkyl)₂, and N(Aryl)₂;
   wherein the carbon atoms of the aforementioned groups are unsubstituted or substituted by one or more substituents selected from —CN, -halogen, —SH, —C(═O)H, —C(═O)OH, C₁-C₆-alkyl, C₁-C₆-haloalkyl, —O(C₁-C₆-alkyl), —C(═O)(C₁-C₆-alkyl), —C(═O)O(C₁-C₆-alkyl), and phenyl;

In a more preferred embodiment,
Z is in each case independently —NCS or —NH₂.

As indicated above, A₁ may in one embodiment be a bridging group comprising from one to three separate straight or branched, saturated or unsaturated carbon-based chains including from 1 to 12 carbon atoms, wherein the carbon-based chains are free of or comprise one to ten, same or different groups selected from —O—, —S—, —NH—, —NR₁—, —C(═O)NH—, —NHC(═O)—, —C(═O)NR₁—, —NR₁C(═O)— and —C(═O)—.

In a preferred embodiment,
A₁ is a bridging group comprising from one to three separate straight or branched, saturated or unsaturated carbon-based chains including from 1 to 12 carbon atoms, wherein the carbon-based chains are free of or comprise one to ten, same or different groups selected from —NR₁—, —C(═O)NH—, —NHC(═O)—, —C(═O)NR₁—, —NR₁C(═O)— and —C(═O)—.

In connection with the above embodiments regarding A₁, it is preferred that
R₁ is in each case independently selected from C₁-C₆-alkyl, and from the option of representing one of the one or two groups -L-Z.

Preferably, R₁ in each case represents one of the one or two groups -L-Z.

As indicated above, A₁ may in another embodiment be a bridging chelate moiety -Che₃- as defined above. In this connection, it is preferred that Ar₁ is any one of the above defined groups. Particularly preferably, Ar₁ is the following group:

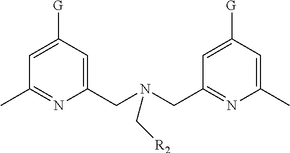

Furthermore, it is preferred in connection with -Che₃- and the preferred option regarding Ar₁ as defined above that
R₂ is in each case independently selected from —C(═O)O⁻, —P(═O)O₂²⁻, P(═O)MeO⁻, —P(═O)PhO⁻, and the C₁-C₆-alkyl esters thereof, and from the option of representing one of the one or two groups -L-Z;
R₃ is in each case independently selected from the bridging groups —C(═O)NH—, —C(═O)NH—(C₁-C₆-alkylene)-NHC(═O)—, —C(═O)NH—(C₁-C₆-alkylene)-O—, —C(═O)NH—(C₁-C₆-alkylene)-C(═O)NH—, —C(═O)NH—(C₁-C₆-alkylene)-S—, —C(═O)NH—(C₁-C₆-alkylene)-NHC(═O)—(C₁-C₆-alkylene)-NHC(═O)—, —C(═O)NH—(C₁-C₆-alkylene)-NHC(═O)—(C₁-C₆-alkylene)-O—, —C(═O)NH—(C₁-C₆-alkylene)-NHC(═O)—(C₁-C₆-alkylene)-C(═O)NH—, —C(═O)NH—(C₁-C₆-alkylene)-NHC(═O)—(C₁-C₆-alkylene)-S—, —C(═O)NH—(C₁-C₆-alkylene)-O—(C₁-C₆-alkylene)-NHC(═O)—, —C(═O)NH—(C₁-C₆-alkylene)-O—(C₁-C₆-alkylene)-O—, —C(═O)NH—(C₁-C₆-alkylene)-O—(C₁-C₆-alkylene)-S—, —C(═O)NH—(C₁-C₆-alkylene)-O—(C₁-C₆-alkylene)-C(═O)NH—, —C(═O)NH—(C₁-C₆-alkylene)-C(═O)NH—(C₁-C₆-alkylene)-NHC(═O)—, —C(═O)NH—(C₁-C₆-alkylene)-C(═O)NH—(C₁-C₆-alkylene)-O—, —C(═O)NH—(C₁-C₆-alkylene)-C(═O)NH—(C₁-C₆-alkylene)-S—, —C(═O)NH—(C₁-C₆-alkylene)-C(═O)NH—(C₁-C₆-alkylene)-C(═O)NH—, and —P(═O)O₂²⁻.

More preferably,
R₂ is in each case —C(═O)O⁻;
R₃ is in each case selected from the bridging groups —C(═O)NH—, —C(═O)NH—(C₁-C₆-alkylene)-NHC(═O)—, —C(═O)NH—(C₁-C₆-alkylene)-O—, —C(═O)NH—(C₁-C₆-alkylene)-C(═O)NH—, —C(═O)NH—(C₁-C₆-alkylene)-S—.

Furthermore, it is preferred in connection with -Che₃- and the preferred option regarding Ar₁ as defined above that
G is in each case independently selected from i) a conjugating group, ii) a single bond, and iii) hydrogen;
   wherein each conjugating group comprises 1, 2, or 3 moieties selected from —CH═CH—, —C≡C—, —C(═O)—, and biradicals of 5 to 10-membered aromatic or heteroaromatic monocyclic or bicyclic rings, wherein the heteroaromatic ring contains one or more, same or different heteroatoms N, O, or S, and wherein the aromatic or heteroaromatic monocyclic or bicyclic rings are unsubstituted or substituted by 1 to 5 same or different substituents R₅, wherein R₅ is defined as above;
   wherein each conjugating group, if present in a terminal position, may further comprise a terminal group, which is selected from the group consisting of —H, -halogen, —CN, —CH₃, and from the option of representing one of the one or two groups -L-Z.

Preferably,
G is in each case independently selected from i) a conjugating group, ii) a single bond, and iii) hydrogen;
   wherein each conjugating group comprises 1, 2, or 3 moieties selected from —CH═CH—, —C(═O)—, phenylene, biphenylene, naphthylene, pyridylene, pyrazinylene, pyrimidinylene, pyridazinylene, furylene, thienylene, pyrrolylene, imidazolylene, pyrazolylene, thiazolylene, isothiazolylene, oxazolylene, isoxazolylene, fyrazanylene, 1,2,4-triazol-3,5-ylene, and oxadiazolylene, wherein the aromatic or heteroaromatic monocyclic or bicyclic rings are unsubstituted or substituted by 1 to 5 same or different substituents R₅.

More preferably,
G is in each case independently selected from i) a conjugating group, ii) a single bond, and iii) hydrogen;
   wherein each conjugating group is independently selected from phenylene-C≡C—, phenylene, thienylene, and furylene.

As indicated above, Che₁ and Che₂ are independently selected from the chelate moieties Che I, Che II, Che III, Che IV, Che V, Che VI, and Che VII as defined above. In a preferred embodiment, Che₁ and Che₂ are independently selected from the chelate moieties Che I and Che IV. In this connection with the aforementioned chelate moieties, in particular in connection with the chelate moieties Che I and Che IV, it is preferred that Ar₂ is any one of the above defined groups. Particularly preferably, Ar₂ is the following group:

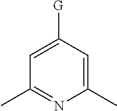

It is to be understood that -Che₁- and Che₂- may be identical or different from each other. Preferably, -Che₁- and Che₂- are identical.

Furthermore, it is preferred in connection with -Che₁- and -Che₂- as well as the above mentioned preferred embodiments in this connection that
R₂ is in each case independently selected from —C(═O)O⁻, —P(═O)O₂²⁻, P(═O)MeO⁻, —P(═O)PhO⁻, and the C₁-C₆-alkyl esters thereof, and from the option of representing one of the one or two groups -L-Z;
R₃ is in each case independently selected from the bridging groups —C(═O)NH—, —C(═O)NH—(C₁-C₆-alkylene)-NHC(═O)—, —C(═O)NH—(C₁-C₆-alkylene)-O—, —C(═O)NH—(C₁-C₆-alkylene)-C(═O)NH—, —C(═O)NH—(C₁-C₆-alkylene)-S—, —C(═O)NH—(C₁-C₆-alkylene)-NHC(═O)—(C₁-C₆-alkylene)-NHC(═O)—, —C(═O)NH—(C₁-C₆-alkylene)-NHC(═O)—(C₁-C₆-alkylene)-O—, —C(═O)NH—(C₁-C₆-alkylene)-NHC(═O)—(C₁-C₆-alkylene)-C(═O)NH—, —C(═O)NH—(C₁-C₆-alkylene)-NHC(═O)—(C₁-C₆-alkylene)-S—, —C(═O)NH—(C₁-C₆-alkylene)-O—(C₁-C₆-alkylene)-NHC(═O)—, —C(═O)NH—(C₁-C₆-alkylene)-O—(C₁-C₆-alkylene)-O—, —C(═O)NH—(C₁-C₆-alkylene)-O—(C₁-C₆-alkylene)-S—, —C(═O)NH—(C₁-C₆-alkylene)-O—(C₁-C₆-alkylene)-C(═O)NH—, —C(═O)NH—(C₁-C₆-alkylene)-C(═O)NH—(C₁-C₆-alkylene)-NHC(═O)—, —C(═O)NH—(C₁-C₆-alkylene)-C(═O)NH—(C₁-C₆-alkylene)-O—, —C(═O)NH—(C₁-C₆-alkylene)-C(═O)NH—(C₁-C₆-alkylene)-S—, —C(═O)NH—(C₁-C₆-alkylene)-C(═O)NH—(C₁-C₆-alkylene)-C(═O)NH—, and —P(═O)O₂²⁻;
R₄ is in each case independently selected from —CH₂N(CH₂C(═O)O⁻)₂, —CH₂N(CH₂P(═O)O₂²⁻)₂, —CH₂N(CH₂P(═O)MeO⁻)₂, —CH₂N(CH₂P(═O)PhO⁻)₂, and from the options defined for R₂.

More preferably,
R₂ is in each case —C(═O)O⁻;
R₃ is in each case selected from the bridging groups —C(═O)NH—, —C(═O)NH—(C₁-C₆-alkylene)-NHC(═O)—, —C(═O)NH—(C₁-C₆-alkylene)-O—, —C(═O)NH—(C₁-C₆-alkylene)-C(═O)NH—, —C(═O)NH—(C₁-C₆-alkylene)-S—;
R₄ is in each case —C(═O)O⁻.

Furthermore, it is preferred in connection with -Che₁- and -Che₂- as well as the above mentioned preferred embodiments in this connection that
G is in each case independently selected from i) a conjugating group, ii) a single bond, and iii) hydrogen;
   wherein each conjugating group comprises 1, 2, or 3 moieties selected from —CH═CH—, —C≡C—, —C(═O)—, and biradicals of 5 to 10-membered aromatic or heteroaromatic monocyclic or bicyclic rings, wherein the heteroaromatic ring contains one or more, same or different heteroatoms N, O, or S, and wherein the aromatic or heteroaromatic monocyclic or bicyclic rings are unsubstituted or substituted by 1 to 5 same or different substituents R₅, wherein R₅ is defined as above;
   wherein each conjugating group, if present in a terminal position, may further comprise a terminal group, which is selected from the group consisting of —H, -halogen, —CN, —CH₃, and from the option of representing one of the one or two groups -L-Z.

Preferably,
G is in each case independently selected from i) a conjugating group, ii) a single bond, and iii) hydrogen;
   wherein each conjugating group comprises 1, 2, or 3 moieties selected from —CH═CH—, —C≡C—, —C(═O)—, phenylene, biphenylene, naphthylene, pyridylene, pyrazinylene, pyrimidinylene, pyridazinylene, furylene, thienylene, pyrrolylene, imidazolylene, pyrazolylene, thiazolylene, isothiazolylene, oxazolylene, isoxazolylene, fyrazanylene, 1,2,4-triazol-3,5-ylene, and oxadiazolylene, wherein the aromatic or heteroaromatic monocyclic or bicyclic rings are unsubstituted or substituted by 1 to 5 same or different substituents R₅.

More preferably,
G is in each case independently selected from i) a conjugating group, ii) a single bond, and iii) hydrogen;
   wherein each conjugating group is independently selected from phenylene-C≡C—, phenylene, thienylene, and furylene.

The conjugating groups in the compounds of the present invention may be modified by a hydrophilic group as defined in connection with R₅, R₆, and R₇. Examples of hydrophilic groups are mono- and oligosaccharides, such as monosaccharides and disaccharides, oligoalkylene glycols (e.g. those having 1-20 repeating units) such as oligoethylene glycol and oligopropylene glycol, etc. In a preferred embodiment, the hydrophilic group is selected from monosaccharides, disaccharides, —(CH₂)₁₋₃—O—(CH₂CH₂O)₀₋₅—H, —(CH₂)₁₋₃—O—(CH₂CH₂O)₀₋₅—C₁₋₄-alkyl, —O—(CH₂CH₂O)₁₋₆—H, and —O—(CH₂CH₂O)₁₋₆—C₁₋₄-alkyl, in particular monosaccharides.

In the present context, the term “monosaccharide” is intended to mean C₅-C₇ carbohydrates being either in the acyclic or in cyclic form. Examples of monosaccharides are C₆ carbohydrates, e.g. those selected from

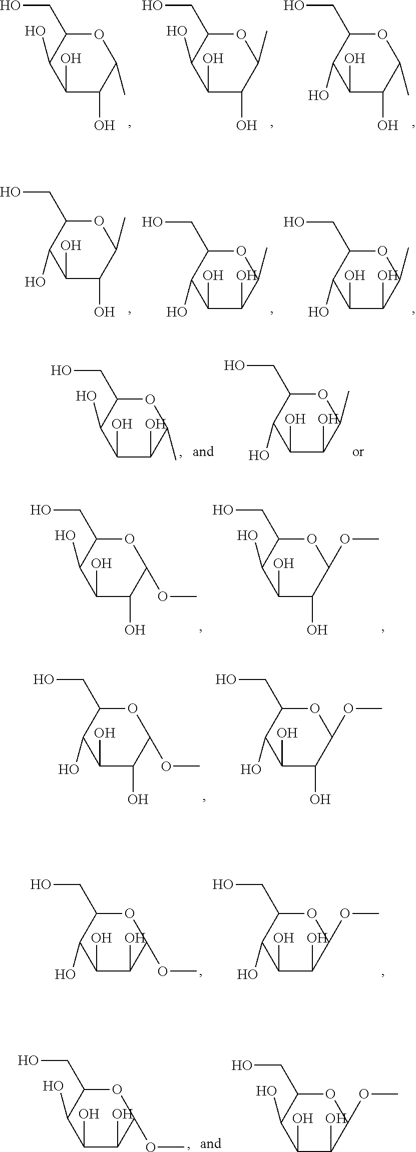

In the present context, the term “disaccharide” is intended to mean two monosaccharides (cf. above) linked together, preferably via glycosidic bonds.

It is to be understood that the hydrophilic group may also comprise a spacer, i.e. a distance-making biradical, such as the ones defined in connection with the group L.

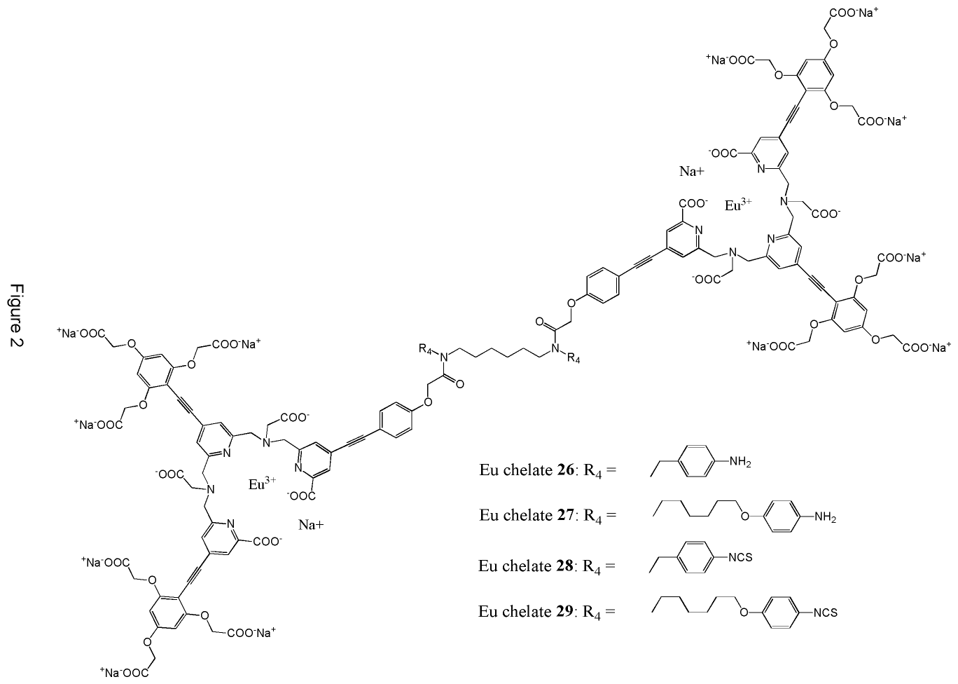

In a particularly preferred embodiment, the compound of formula (I) is any one of the compounds 6, 7, 26, 27, 28, 29, 35, 36, 45, 46, 47, 48, 56, 57, 58, 59, 65, and 66 as defined herein (see FIGS. 2, 4, 6, 8, 9, and Schemes 2 and 13).

As indicated above, the present invention also relates to compounds of formula (II), also referred to as ligands and ligand esters, which may be used as precursors for the compounds of formula (I), i.e. the (lanthanide) chelates. The above defined preferred embodiments regarding groups and substituents of the compounds of formula (I) are also preferred for the compounds of formula (II). However, the chelate moieties do not contain a lanthanide ion.

In yet another aspect, the present invention relates to a detection agent comprising a biospecific binding reactant conjugated to a compound of formula (I) or a salt thereof or a compound of formula (II) or a salt thereof. The detection agent is a detectable molecule comprising a biospecific binding reactant conjugated to a luminescent lanthanide chelate of formula (I) or a precursor of formula (II) of the present invention. Conjugation, i.e. the formation of a covalent bond, is typically achieved by means of the reactive group Z of said chelate. The biospecific binding reactant should be capable of specifically binding an analyte of interest for the purpose of quantitative or qualitative analysis of said analyte in a sample.

Examples of biospecific binding reactants are those selected from an antibody, an antigen, a receptor ligand, a specific binding protein, a DNA probe, a RNA probe, an oligopeptide, an oligonucleotide, a modified oligonucleotide (e.g. a locked nucleic acid (LNA) modified oligonucleotide), a modified polynucleotide (e.g. an LNA modified polynucleotide), a protein, an oligosaccaride, a polysaccharide, a phospholipid, a PNA, a steroid, a hapten, a drug, a receptor binding ligand, and lectin. In a preferred embodiment, the biospecific binding reactant is selected from antibodies, e.g. Troponin I antibodies (anti-TnI).

In another aspect, the present invention relates to a method of detecting an analyte in a biospecific binding assay, said method comprising the steps of:

a) forming a complex between the analyte and the compound of formula (I) or formula (II) or the detection agent of the invention;
b) exciting said complex with a radiation having an excitation wavelength of the compound of formula (I) or formula (II) or the detection agent of the invention, thereby forming an excited complex; and
c) detecting emission radiation emitted from said excited complex.

The method follows conventional assay steps as will be evident for the skilled person. Preferred excitation wavelengths are in the range of from 320-370 nm. The skilled person is aware that the exact excitation wavelengths depend on the specific structure of the ligand. Further, the skilled person is aware that the emission wavelengths are specific for the used lanthanide (Tb³⁺, Eu³⁺, Dy³⁺, Sm³⁺), In case of Eu³⁺, which is preferred according to the invention, the preferred measured emission wavelength is in the range of from 610-620 nm, preferably about 615 nm.

In yet another aspect, the present invention relates to a method of labelling a biospecific binding reactant with a compound of the invention, comprising the steps of

a) providing a biospecific binding reactant; and
b) conjugating the biospecific binding reactant with the compound of formula (I) or formula (II).

The resulting compound may be a detection agent of the invention. The conjugation may occur via the reaction group Z of the compound of formula (I) or formula (II).

In another aspect, the present invention relates to the use of a compound of formula (I) or formula (II) of the invention for the in vitro detection of an analyte in a sample. The present invention thus also relates to the use of a detection agent of the invention in a specific bioaffinity based binding assay, e.g. utilizing time-resolved fluorometric determination of a specific luminescence. In one embodiment, the specific bioaffinity based binding assay is a heterogeneous immunoassay, a homogenous immunoassay, a DNA hybridization assay, a receptor binding assay, an immunocytochemical or an immunohistochemical assay.

In another aspect, the present invention relates to the use of a compound of formula (I) or formula (II) of the invention or the detection agent of the invention in bio-imaging applications. Such a use is particularly advantageous if the compound of formula (I) or formula (II) of the invention is a molecule with a neutral net charge or almost neutral net charge (i.e. the molecule comprises an overall net charge of from −3 to +5). This of course depends on the selection of the substituents. However, the compounds of the invention or the detection agent of the invention may for example be used as a contrasting agent. The contrasting agent may e.g. be used in MM or PET applications. The compounds of the invention or the detection agent of the invention may further be used for microscopy applications, e.g. in cell culture experiments, such as in confocal laser scanning microscopy and or hybridization experiments.

Still another aspect of the invention relates to a solid support material conjugated with a compound of formula (I) or formula (II) of the invention or the detection agent of the invention. The compound or the detection agent of the invention is typically immobilized to the solid support material either covalently or non-covalently.

In some embodiments, the solid support material is selected from a nanoparticle, a microparticle, a slide, a plate, and a solid phase synthesis resin.

The present invention is further illustrated by the following examples.

### EXAMPLES

The following non-limiting examples are aimed to further demonstrate the invention.

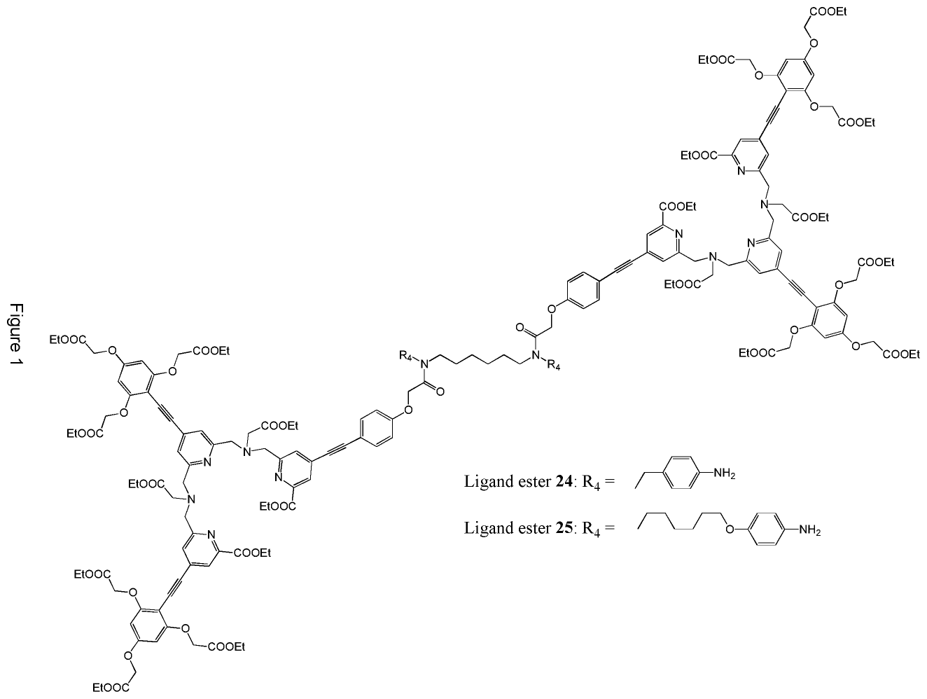

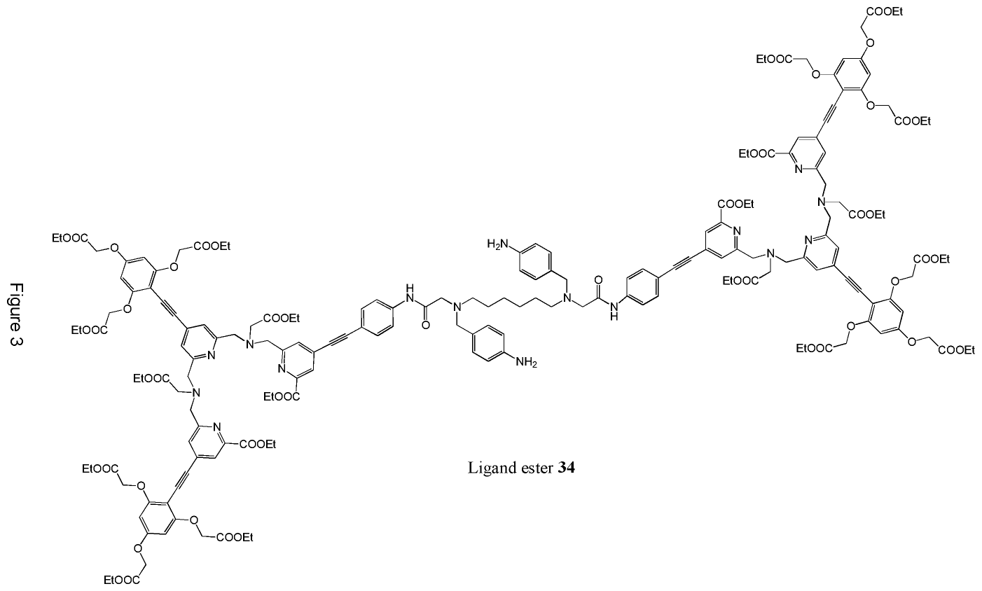

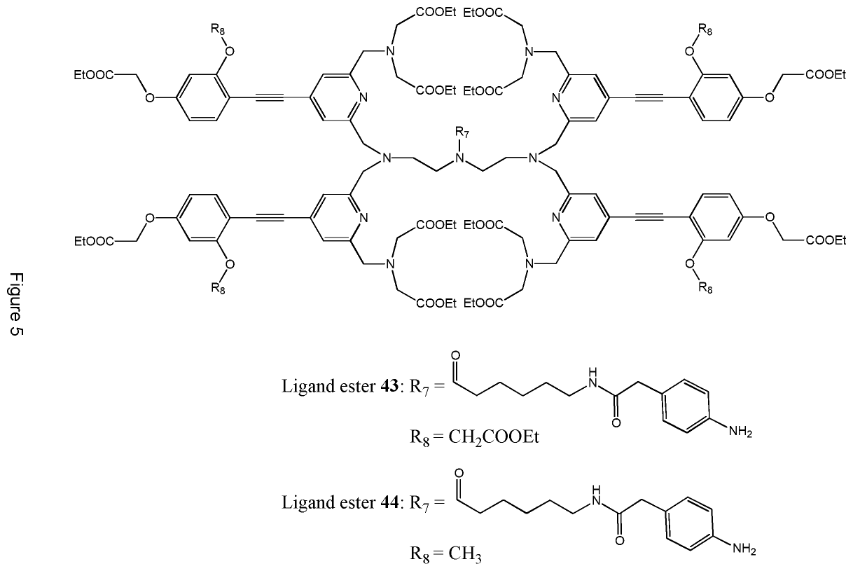

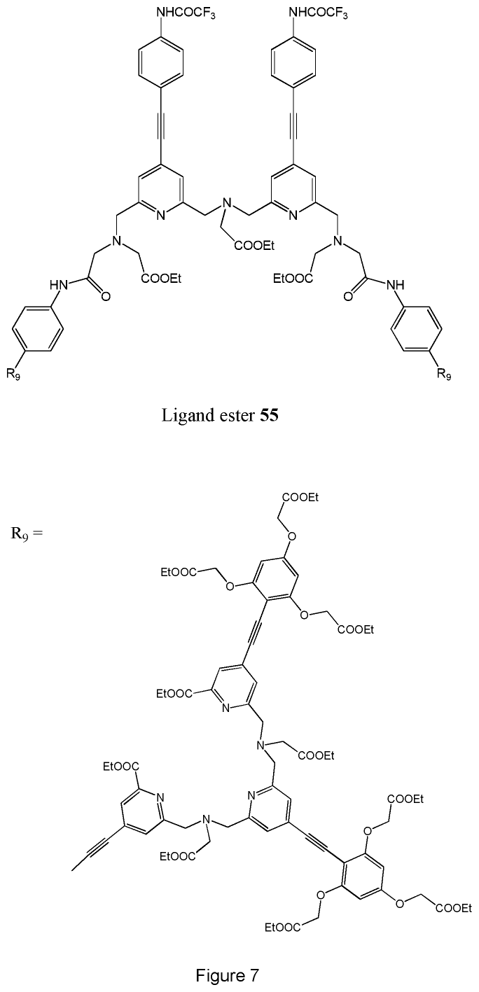

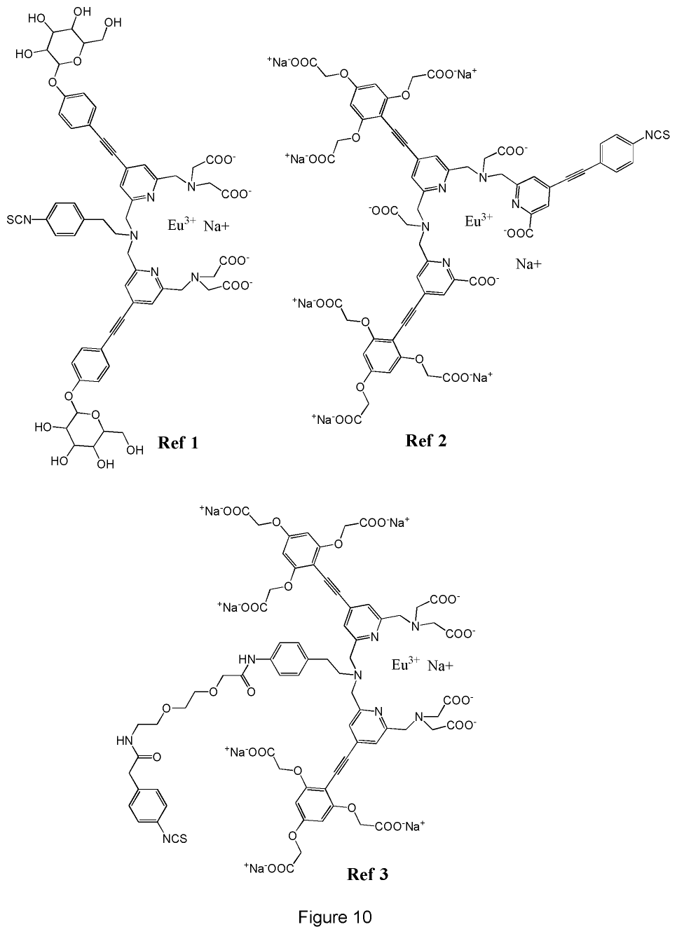

The structures and synthetic routes employed are presented in Schemes 1-13 as provided at the end of the experimental part. Furthermore, reference is made to FIGS. 1-9. FIG. 10 provides the chemical structures of reference chelates Ref 1-3 used in this application.

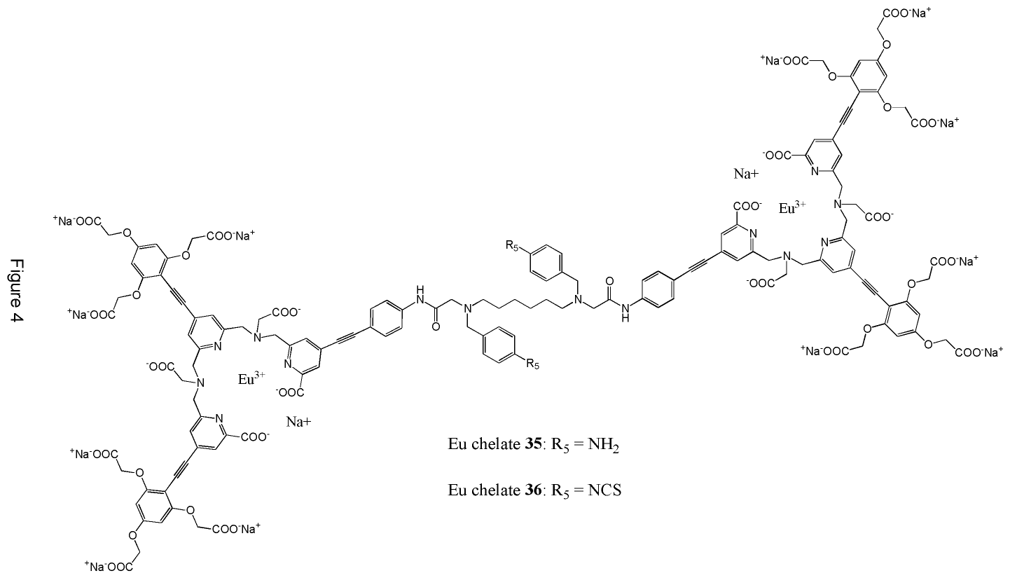

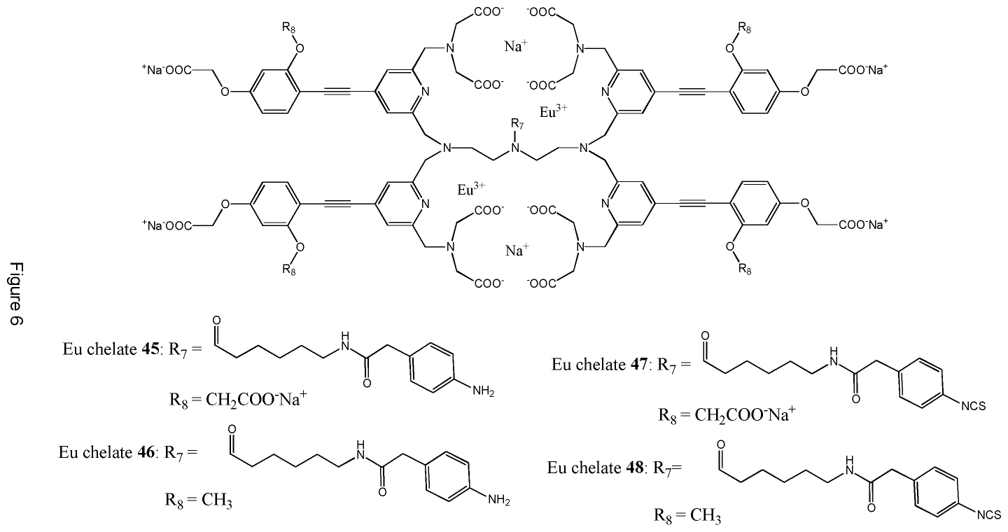

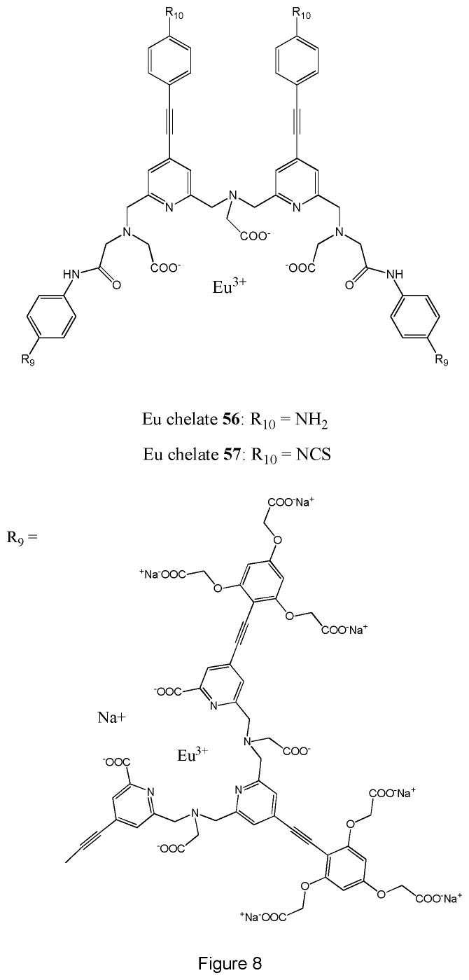

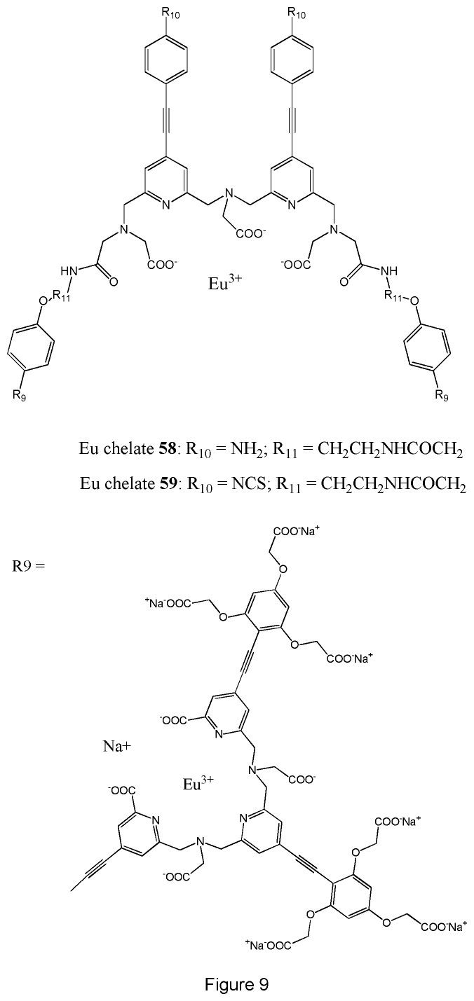

In compounds 6 and 7 (Scheme 2), in compounds 65 and 66 (Scheme 13) as well as in compounds 45-48 (FIG. 6), both Eu(III) ions form equal coordination bonds and one of the two Eu(III) ions forms coordination bonds with two pyridine nitrogen atoms, three tertiary nitrogen atoms connected to the pyridine rings with CH₂ bridges and four negatively charged oxygen atoms of the carboxylic groups. In compounds 26-29 (FIG. 2) and in compounds 35 and 36 (FIG. 4), both Eu(III) ions form equal coordination bonds and one of the two Eu(III) ions forms coordination bonds with three pyridine nitrogen atoms, two tertiary nitrogen atoms connected to the pyridine rings with CH₂ bridges and four negatively charged oxygen atoms of the carboxylic groups (two carboxylic groups between the pyridine moieties and two carboxylic groups in pyridine rings). In compounds 56 and 57 (FIG. 8) and in compounds 58 and 59 (FIG. 9) one of the three Eu(III) ion forms coordination bonds with two pyridine nitrogen atoms, two tertiary nitrogen atoms connected to the pyridine rings with CH₂ bridges, two negatively charged oxygen atoms of the carboxylic groups and two amide groups (—CONH—); the rest two Eu(III) ions form equal coordination bonds and one of the rest two Eu(III) ions forms coordination bonds with three pyridine nitrogen atoms, two tertiary nitrogen atoms connected with pyridine rings with CH₂ bridges and four negatively charged oxygen atoms of the carboxylic groups (two carboxylic groups between the pyridine moieties and two carboxylic groups in pyridine rings).

¹H-NMR spectra were recorded with Bruker AVANCE DRX 500 and 600 MHz. Tetramethyl silane was used as internal reference. Mass spectra were recorded PerSeptive Biosystems Voyager DE-PRO MALDI-TOF instrument using α-cyano-4-cinnamic acid matrix. UV-Vis spectra were recorded on Pharmacia Ultrospec 3300 pro. Fluorescence efficiencies were determined with Perkin-Elmer Wallac Victor plate fluorometer. Eu-content of Eu-chelates and labelled antibodies were measured by using ICP-MS instrument, PerkinElmer 6100 DRC Plus, in quantitative mode. The excitation, emission spectra and decay times were recorded on a Varian Cary Eclipse fluorescence spectrometer.

Conditions for HPLC purification runs: Reversed phase HPLC (RP-18 column). The solvents were A: triethyl ammonium acetate buffer (20 mM, pH7) and B: 50% acetonitrile in triethyl ammonium acetate buffer (20 mM, pH7). The gradient was started from 5% of solvent B and the amount of solvent B was linearly raised to 100% in 30 minutes.

Column chromatography was performed with columns packed with silica gel 60 (Merck). FC=Flash chromatography, RT=room temperature.

### Example 1. Synthesis of Compound 2

A mixture of compound 1 (0.23 g, 0.51 mmol; Takalo, H., et al., Helv. Chim. Acta 79(1996)789)), tert-butyl (6-aminohexyl)carbamate (55 mg, 0.25 mmol), dry K₂CO₃ (0.28 g, 2.04 mmol) and dry MeCN (10 ml) under argon was stirred at RT overnight. After filtration and washes with MeCN, the product (0.24 g, 100%) was used for the next step without further purifications. ¹H NMR (CDCl₃, δ ppm): 7.71 (2H, d, J=0.95 Hz), 7.59 (2H, d, J=0.95 Hz), 4.56 (2H, s), 4.15 (8H, q, J=7.15 Hz), 4.02 (4H, s), 3.78 (4H, s), 3.60 (8H, s), 3.14-3.01 (2H, m), 2.62-2.49 (2H, m), 1.59-1.49 (2H, m), 1.49-1.41 (6H, m), 1.27 (12H, t, J=7.15 Hz). ¹³C NMR (CDCl₃, δ ppm): 170.93, 160.44, 160.30, 160.10, 134.17, 123.49, 124.28, 78.90, 60.93, 59.74, 54.94, 54.43, 40.42, 29.95, 28.34, 26.85, 26.53, 14.15. MALDI TOF-MS mass: calculated (M+H⁺) 957.30, 959.30 961.30; found 957.15, 959.16, 961.07.

### Example 2. Synthesis of Compound 3

A mixture of the compound 2 (0.23 g, 0.24 mmol) and N-(4-ethynylphenyl)-2,2,2-trifluoroacetamide (0.12 g, 0.58 mmol; Sund, H., et al. Molecules 22(2017)1807) in dry TEA (1 ml) and THF (2 ml) was de-aerated with argon. After addition of bis(triphenylphosphine)palladium(II) chloride (10 mg, 14 μmol) and CuI (6 mg, 28 μmol), the mixture was stirred overnight at 55° C. After evaporation to dryness, the product (0.27 g, 91%) was purified by FC (silica gel, 5% EtOH/CH₂Cl₂). ¹H NMR (CDCl₃, δ ppm): 8.84 (2H, s), 7.65 (2H, s), 7.59 (2H, s), 7.56 (4H, d, J=8.70 Hz), 7.50 (4H, d, J=8.70 Hz), 4.17 (8H, q, J=7.10 Hz), 4.04 (4H, s), 3.76 (4H, s), 3.61 (8H, s), 3.08-3.01 (2H, m), 2.57-2.50 (2H, m), 1.55-1.47 (2H, m), 1.47-1.38 (6H, m), 1.26 (12H, t, J=7.1 Hz). ¹³C NMR (CDCl₃, δ ppm): 171.05, 160.31, 158.40, 155.90, 155.20, 154.93, 154.63, 154.38, 135.60, 132.67, 131.85, 123.08, 122.61, 121.11, 120.23, 118.98, 116.70, 114.41, 111.71, 92.03, 88.07, 78.92, 60.59, 60.51, 59.70, 54.89, 53.33, 40.45, 29.60, 28.30, 26.97, 26.55, 14.14. MALDI TOF-MS mass: calculated (M+H⁺) 1223.53; found 1223.90.

### Example 3. Synthesis of Compound 4

The mixture of the compound 3 (0.25 g, 0.19 mmol) in TFA (2.8 ml) was stirred for 2 h at RT and evaporated to dryness. The residue was co-evaporated from diethyl ether (2×20 ml) and dissolved in dry MeCN (6 ml). After an addition of DIPEA (0.66 ml, 3.8 mmol) and a solution of compound 1 (0.17 g, 0.38 mmol) in dry MeCN (6 ml), the mixture was stirred for 67 h at RT. After evaporation to dryness, the residue was dissolved in CH₂Cl₂ (30 ml), washed with H₂O (3×15 ml) and dried with Na₂SO₄. The product (0.31 g, 88%) was purified by (silica gel, 10% EtOH/CH₂Cl₂). ¹H NMR (CDCl₃, δ ppm): 9.04 (2H, s), 7.66 (2H, s), 7.63 (2H, s), 7.58 (2H, s), 7.54 (2H, d. J=8.60 Hz), 7.49 (2H, s), 7.45 (2H, d, J=8.60 Hz), 4.15 (8H, q, J=7.2 Hz), 4.15 (8H, q, J=7.2 Hz), 4.04 (4H, s), 3.96 (4H, s), 3.75 (4H, s), 3.66 (4H, s), 3.61 (8H, s), 3.57 (8H, s), 2.58-2.51 (2H, m) 2.51-2.42 (2H, m), 1.57-1.46 (4H, m), 1.27 (12H, t, J=7.2 Hz), 1.25 (12H, t, J=7.2 Hz), 1.20-1.10 (4H, m). ¹³C NMR (CDCl₃, δ ppm): 171.07, 171.02, 160.84, 160.38, 159.87, 158.40, 155.82, 155.53, 155.22, 154.91, 135.72, 134.08, 132.58, 131.94, 124.26, 123.97, 123.07, 122.57, 121.12, 120.03, 119.04, 116.73, 114.44, 112.15, 92.09, 88.02, 60.67, 60.60, 60.54, 60.48, 59.82, 59.70, 59.46, 59.24, 55.40, 54.97, 54.88, 54.56, 29.59, 27.25, 14.15, 14.12. MALDI TOF-MS mass: calculated (M+H⁺) 1865.58, 1866.58; found 1864.34, 1866.53.

### Example 4. Synthesis of Compound 5

A mixture of the compound 4 (0.29 g, 0.155 mmol), triethyl 2,2′,2″-{[4-(ethynyl)benzene-1,3,5-triyl]-tris(oxy)triacetate (0.15 g, 0.373 mmol; Sund, H., et al. Molecules 22(2017)1807) in dry TEA (1 ml) and THF (2 ml) was de-aerated with argon. After addition of bis(triphenylphosphine)palladium(II) chloride (10 mg, 14 μmol) and CuI (5 mg, 28 μmol), the mixture was stirred for 24 h at 55° C. After evaporation to dryness, the product (0.32 g, 82%) was purified by FC (silica gel, 5% EtOH/CH₂Cl₂). MALDI TOF-MS mass: calculated (M+2H⁺); 2522.59, found 2523.66.

### Example 5. Synthesis of Compound 6

Compound 5 (0.30 g, 0.119 mmol) in 0.5 M KOH/EtOH (18.5 ml) was stirred for 30 min at RT. After an addition of H₂O (9 ml), the mixture was further stirred at RT for 2 h. After EtOH evaporation and an additional overnight stirring at RT, the pH was adjusted to 6.5 by addition of 6 M HCl. EuCl₃ (92 mg, 0.250 mmol) in water (1 ml) was added within 5 min and the pH was maintained at 6.0-6.5 with suitable additions of solid NaHCO₃. After stirring overnight at RT, the pH was adjusted to 8.5 with 1M NaOH. The precipitate was removed by centrifugation and the supernatant evaporated to dryness. The product was purified by HPLC. Yield: 0.26 g (72%). R_{f}(HPLC): 16.5 min. UV: 352 nm.

### Example 6. Synthesis of Compound 7

Compound 6 (0.112 g, 37 μmol) in H₂O (1.4 ml) was added within 5 min to a mixture of CSCl₂ (29 μl, 0.52 mmol) and NaHCO₃ (49 mg, 0.59 mmol) and CHCl₃ (1.4 ml). After stirring for 20 min at RT, the aqueous phase was washed with CHCl₃ (3×1.4 ml). The product was precipitated with acetone, centrifuged and washed with acetone. The product was used for the antibody labelling without any further purifications.

### Example 7. Synthesis of Compound 8

A mixture of 4-nitrophenol (1.39 g, 10 mmol), 1,6-diaminohexane (1.54 ml, 10 mmol), dry Na₂CO₃ (4.24 g, 40 mmol) and dry DMF (25 ml) was for 2.5 h at 100° C. After evaporation to dryness and an addition of CH₂Cl₂ (50 ml), the mixture was washed with H₂O (25 ml), 2M NaOH (25 ml), 5% NaHCO₃ (25 ml), H₂O (25 ml) and dried with Na₂SO₄ (25 ml). The product (0.84 g, 28%) was purified by FC (silica gel, 10% EtOAc/petroleum ether). ¹H NMR (D₆-DMSO, δ ppm): 8.20 (2H, d, J=9.3 Hz), 7.13 (2H, d, J=9.3 Hz), 4.12 (2H, t, J=6.5 Hz), 3.54 (2H, t, J=6.7 Hz), 1.86-1.79 (2H, m), 1.79-1.72 (2H, m), 1.47-1.43 (4H, m). ¹³C NMR (D₆-DMSO, δ ppm): 164.49, 141.17, 126.34, 115.44, 68.99. 35.52, 32.62, 28.69, 27.69, 23.99. MALDI TOF-MS mass: calculated (M+H⁺); 302.04, 304.04, found 301.65, 303.65.

### Example 8. Synthesis of Compound 9

After stirring of a mixture of N,N′-(hexane-1,6-diyl)bis(2,2,2-trifluoroacetamide) (0.98 g, 3.18 mmol) and NaH (0.27 g, 6.68 mmol; 60% in oil) in dry DMF (10 ml) for 30 min at RT, 3-nitrobenzylbromide (1.44 g, 6.67 mmol) was added. The reaction mixture was stirred for overnight at RT, and evaporated to dryness. After an addition of H₂O (50 ml), the solid crude product was filtrated and washed with H₂O. A dried solid material was suspended in 30% EtOAc/petroleum ether, filtrated and washed with EtOAc/petroleum ether. Yield: 1.67 g (91%). MALDI TOF-MS mass: calculated (M+H⁺) 579.17; found 579.02.

### Example 9. Synthesis of Compound 10

After stirring of a mixture of N,N′-(hexane-1,6-diyl)bis(2,2,2-trifluoroacetamide) (0.42 g, 1.36 mmol) and NaH (0.11 g, 2.86 mmol; 60% in oil) in dry DMF (5 ml) for 30 min at RT, a solution of the compound 8 (0.82 g, 2.72 mmol) in dry DMF (6 ml) was added. The reaction mixture was stirred for 22 h at 75° C., and evaporated to dryness. The residue was dissolved in CH₂Cl₂ (40 ml), was washed with H₂O (2×20 ml) and dried with Na₂SO₄. The product (0.51 g, 50%) was purified by FC (silica gel, from 30% to 40% EtOAc/petroleum ether). ¹H NMR (D₆-DMSO, δ ppm): 9.38 (2H, s), 8.20 (4H, d, J=9.1 Hz), 7.13 (4H, d, J=9.1 Hz), 4.12 (4H, t, J=5.8 Hz), 3.38-3.30 (4H, m), 3.17 (4H, q, J=6.5 Hz), 1.80-1.70 (4H, m), 1.64-1.50 (8H, m), 1.50-1.40 (8H, m), 1.35-1.20 (12H, m). ¹³C NMR (D₆-DMSO, δ ppm): 154.49, 156.75, 156.47, 155.89, 155.61, 141.18, 126.35, 120.37, 118.96, 115.78, 115.44, 113.51, 68.99, 68.96, 47. 42, 46.83, 28.70, 28.56, 28.53, 28.51, 26.66, 26.64, 26.30, 26.26, 26.19, 26.15, 26.07, 25.91, 25.49, 25.43, MALDI TOF-MS mass: calculated (M+H⁺) 751.32; found 751.33.

### Example 10. Synthesis of Compound 11

After stirring of a mixture of the compound 9 (1.46 g, 2.52 mmol) in 0.5M KOH (20 ml) and CH₂Cl₂ (30 ml) overnight at RT, a second set of CH₂Cl₂ (30 ml) was added, and the mixture was washed with H₂O (2×20 ml) and dried with Na₂SO₄. Yield: 0.97 g (100%). ¹H NMR (D₆-DMSO, δ ppm): 8.20 (2H, s), 8.07 (2H, dd, J=7.9 and 1.6 Hz), 7.77 (2H, d, J=7.9 Hz), 7.59 (2H, t, J=7.9 Hz), 3.80 (4H, s), 2.46 (4H, t, J=7.0 Hz), 1.48-1.37 (4H, m), 1.32-1.24 (4H, m). ¹³C NMR (D₆-DMSO, δ ppm): 148.20, 144.32, 134.91, 129.83, 122.53, 121.76, 52.38, 48.98, 29.91, 27.16. MALDI TOF-MS mass: calculated (M+H⁺) 387.21; found 386.81.

### Example 11. Synthesis of Compound 12

Compound 12 was synthesized from compound 10 using a method analogous to the synthesis described in example 10. Yield: 89%. ¹H NMR (D₆-DMSO, δ ppm): 8.19 (4H, d, J=9.3 Hz), 7.13 (4H, d, J=9.3 Hz), 4.14-4.08 (4H, m), 2.48-2.43 (8H, m), 1.78-1.70 (4H, m), 1.46-1.30 (16H, m), 1.29-1.22 (4H, m). ¹³C NMR (D₆-DMSO, δ ppm): 164.53, 141.20, 126.31, 115.45, 69.11, 49.87, 49.78, 30.05, 29.94, 28.86, 27.34, 27.00, 26.81, 25.79. MALDI TOF-MS mass: calculated (M+H⁺) 559.35; found 559.18.

### Example 12. Synthesis of Compound 13

N-hydroxysuccinimide (0.19 g, 1.69 mmol) and N,N-dicyclohexylcarbodiimide (0.35 g, 1.69 mmol) was added to a solution of 2-(4-iodophenoxy)acetic acid (0.47 g, 1.69 mmol) in dry 1,4-dioxane (5 ml). After stirring for 2.5 h at RT, a solution of the compound 11 (0.33 g, 0.85 mmol) in dry 1,4-dioxane (2.5 ml) was added, and the mixture was stirred for 24 h at RT. The mixture was filtrated, the solid material washed with 1,4-dioxane (4×5 ml) and the filtrate was evaporated to dryness. The residue was dissolved in CH₂Cl₂ (40 ml), washed with 10% NaOH (20 ml), 5% NaHCO₃ (20 ml), H₂O (20 ml) and dried with Na₂SO₄. The product (0.42 g, 55%) was purified by FC (silica gel, from 0% to 1% EtOH/CH₂Cl₂). MALDI TOF-MS mass: calculated (M+H⁺) 907.08; found 906.94.

### Example 13. Synthesis of Compound 14

After an addition of DIPEA (0.24 ml, 1.36 mmol) and PyAOP (0.35 g, 0.75 mmol) into a solution of compound 12 (0.19 g, 0.34 mmol) and 2-(4-iodophenoxy)acetic acid (0.19 g, 0.68 mmol) in dry DMF (7.5 ml), the mixture was stirred for 2 h at RT and evaporated to dryness. The product (0.31 g, 84%) was purified by FC (silica gel, from 1% to 2% EtOH/CH₂Cl₂). MALDI TOF-MS mass: calculated (M+Na⁺): 1101.77; found 1101.35.

### Example 14. Synthesis of Compound 15

A mixture of 4-bromo-6-hydroxymethyl-2-carboxyethylpyridine (1.56 g, 6.0 mmol; Takalo, H., et al., Helv. Chim. Acta 79(1996)789)), bis(triphenylphosphine)palladium(II) chloride (84 mg, 0.12 mmol) and CuI (46 mg, 0.24 mmol) in dry TEA (5 ml) and THF (10 ml) was de-aerated with argon. After an addition of trimethylsilylacetylene (1.2 ml, 8.4 mmol), the mixture was stirred overnight at RT. After evaporation to dryness, the product (1.66 g, 100%) was purified by FC (silica gel, from 2% to 5% EtOH/CH₂Cl₂).

### Example 15. Synthesis of Compound 16

A mixture of the compound 15 (0.277 g, 1.0 mmol) and PBr₃ (113 μmol) in dry CHCl₃ (10 ml) was stirred for 3 h at RT. After an addition of CHCl₃ (20 ml), the mixture was neutralized with 5% NaHCO₃ (20 ml), the aqueous phase was extracted with CHCl₃ (10 ml), the combined organic phases were dried with Na₂SO₄. The product (0.27 g, 79%) was purified by FC (silica gel, 5% EtOH/CH₂Cl₂). ¹H NMR (D₆-DMSO, δ ppm): 7.90 (1H, s), 7.86 (1H, s), 4.74 (2H, s), 4.37 (2H, q, J=7.1 Hz), 1.34 (3H, t, J=7.1 Hz), 0.27 (9H, s). ¹³C NMR (D₆-DMSO, δ ppm): 164.03, 158.48, 148.59, 132.59, 129.02, 125.90, 102.05, 101.27, 61.99, 33.74, 14.45, −0.16. MALDI TOF-MS mass: calculated (M+H⁺) 340.04, 342.04; found 339.62, 341.62.

### Example 16. Synthesis of Compound 17

A mixture of 4-bromo-2,6-dibromomethylpyridine (3.50 g, 10 mmol; Takalo, H., et al. Acta Chem, Scand. Ser. B 248(1988)₃₇₃), glycine ethyl ester hydrochloride (14.0 g, 0.10 mmol) and di-isopropylethylamine (35 ml) in dry MeCN (130 ml) was stirred overnight at RT. After evaporation to dryness, the residue was dissolved in CH₂Cl₂ (100 ml), washed with H₂O (3×50 ml), dried with Na₂SO₄, and the product was purified by FC (silica gel from 10:20:70 to 15:30:55 TEA/EtOAc/petroleum ether. Yield 2.48 g (64%). ¹H NMR (CDCl₃, δ ppm): 7.43 (2H, s), 4.20 (4H, q, J=7.2 Hz), 3.91 (4H, s), 3.46 (4H, s), 2.25-2.15 (2H, bs), 1.28 (6H, t, J=7.2 Hz). ¹³C NMR (CDCl₃, δ ppm): 172.00, 160.40, 133.83, 60.75, 54.00, 50.34, 14.18. MALDI TOF-MS mass: calculated (M+H⁺): 388.09, 390.09, found 388.75, 390.75.

### Example 17. Synthesis of Compound 18

4-Bromo-6-bromomethyl-2-carboxyethylpyridine (2.06 g, 6.39 mmol; Takalo, H., et al. Helv. Chim. Acta 79(1996)₇₈₉) was added in small portions to a mixture of the compound 17 (2.49 h, 6.39 mmol), dry K₂CO₃ (3.53 g, 25.6 mmol) in dry MeCN (215 ml) within 2 h at RT. After stirring for 22 h at RT, the mixture was filtrated and the filtrate evaporated to dryness. The product was purified by FC (silica gel 10:25:65 TEA/EtOAc/petroleum ether. Yield 2.03 g (50%). ¹H NMR (CDCl3, δ ppm): 8.12 (1H, d, J=1.7 Hz), 8.07 (1H, d, J=1.7 Hz), 7.55 (1H, d, J=1.5 Hz), 7.43 (1H, d, J=1.5 Hz), 4.46 (2H, q, J=7.2 Hz), 4.19 (4H, q, J=7.2 Hz), 4.09 (2H, s), 3.95 (2H, s), 3.90 (2H, s), 2.3-2.1 (1H, bs), 1.43 (3H, t, J=7.2 Hz), 1.29 (3H, t, J=7.2 Hz), 1.29 (3H, t, J=7.2 Hz). ¹³C NMR (CDCl3, δ ppm): 171.98, 170.81, 164.03, 161.57, 160.41, 159.70, 148.54, 134.26, 133.90, 129.13, 126.95, 124.60, 123.77, 62.16, 60.75, 60.63, 59.61, 59.48, 55.38, 54.00, 50.36, 14.19, 14.17, 14.14. MALDI TOF-MS mass: calculated for (M+H⁺): 629.06, 631.05. 633.06, found 629.54, 631.56, 633.54.

### Example 18. Synthesis of Compound 19

A mixture of compound 18 (0.75 g, 1.19 mmol) and triethyl 2,2′,2″-{[4-(ethynyl)benzene-1,3,5-triyl]-tris(oxy)triacetate (1.17 g, 2.86 mmol) in dry TEA (10 ml) and DMF (20 ml) was de-aerated with argon. After an addition of bis(triphenylphosphine)palladium(II) chloride (32 mg, 48 μmol) and CuI (18 mg, 95 μmol), the mixture was stirred overnight at 55° C. After evaporation to dryness, the product (1.23 g, 80%) was purified by FC (silica gel, from 10:90:0 to 10:88:2 EtOH/CH₂Cl₂/TEA). ¹H NMR (D₆-DMSO, δ ppm): 7.86 (1H, s), 7.78 (1H, s), 7.35 (1H, s), 7.31 (1H, s), 6.25 (2H, s), 6.22 (2H, s), 4.88 (4H, s), 4.86 (4H, s), 4.82 (2H, s) 4.81 (2H, s), 4.34 (2H, q, J=7.1 Hz), 4.20-4.13 (12H, m), 4.09-4.02 (4H, m), 3.96 (2H, s), 3.80 (2H, s), 3.54 (2H, s), 3.35 (2H, s), 1.32 (3H, t, J=7.1 Hz), 1.24-1.14 (24H, m). ¹³C NMR (D₆-DMSO, δ ppm): 172.27, 171.12, 168.55, 168.54, 168.50, 164.66, 161.01, 160.85, 160.68, 160.47, 160.18, 158.43, 147.80, 133.34, 132.55, 127.05, 124.50, 122.53, 121.56, 94.61, 94.26, 93.88, 93.77, 93.41, 93.38, 89.00, 87.20, 65.77, 65.72, 65.38, 61.67, 61.08, 60.26, 59.25, 59.19, 54.93, 53.96, 50.18, 46.04, 14.43, 14.41, 14.38, 14.36. MALDI TOF-MS mass: calculated (M+H⁺) 1285.49; found 1285.26.

### Example 19. Synthesis of Compound 20

A mixture of compound 19 (0.64 g, 0.50 mmol), 16 (0.20 g, 0.60 mmol) and dry K₂CO₃ (0.28 g, 2.0 mmol) in dry MeCN (10 ml) was stirred overnight at RT. The mixture was filtrated, inorganic salt was washed with MeCN and the filtrate was evaporated to dryness. The product (0.54 g, 70%) was purified by FC (silica gel, from 5% to 10% EtOH/CH₂Cl₂). ¹H NMR (D₆-DMSO, δ ppm): 7.85 (1H, d, J=1.0 Hz), 7.78 (1H, d. J=1.0 Hz), 7.77 (1H, s), 7.37 (1H, s), 7.34 (1H, s), 6.24 (2H, s), 6.23 (2H, s), 4.87 (4H, s), 4.85 (4H, s), 4.82 (2H, s), 4.81 (2H, s), 4.36-4.28 (4H, m), 4.20-4.12 (12H, m), 4.07-4.01 (6H, m), 4.01 (2H, s), 3.95 (2H, s), 3.92 (2H, s), 3.53 (2H, s), 3.50 (2H, s), 1.31 (3H, t, J=7.2 Hz), 1.29 (3H, t, J=7.1 Hz), 1.24-1.13 (26H, m), 0.21 (9H, s). ¹³C NMR (D₆-DMSO, δ ppm): 171.13, 171.08, 168.53, 168.49, 164.65, 164.39, 161.05, 160.99, 160.85, 160.62, 160.47, 158.61, 158.52, 147.77, 133.38, 132.65, 131.70, 127.94, 127.06, 124.98, 124.52, 123.03, 122.96, 101.92, 100.89, 94.50, 94.28, 93.89, 93.78, 93.41, 88.99, 87.42, 65.77, 65.73, 65.39, 61.72, 61.65, 61.07, 61.03, 60.27, 60.24, 59.40, 59.37, 59.20, 55.27, 55.21, 54.89, 14.37, −0.24. MALDI TOF-MS mass: calculated (M+H⁺) 1544.60; found 1544.55.

### Example 20. Synthesis of Compound 21

A mixture of compound 20 (1.43 g, 0.922 mmol) and tetrabutylammonium fluoride (0.29 mg, 1.11 mmol) in CH₂Cl₂ (30 ml) was stirred for 70 min at RT. After an addition of CH₂Cl₂ (30 ml), the mixture was washed with 10% aqueous citric acid solution (30 ml), H₂O (30 ml) and dried with Na₂SO₄. The product (1.36 g, 100%) was used for the next step without any further purifications. ¹H NMR (D₆-DMSO, δ ppm): 7.85 (1H, d, J=1.1 Hz), 7.84 (1H, s), 7.83 (1H, s), 7.79 (1H, d, J=1.1 Hz), 7.37 (1H, s), 7.36 (1H, s), 6.24 (2H, s), 6.22 (2H, s), 4.87 (4H, s), 4.86 (4H, s), 4.82 (2H, s), 4.81 (2H, s), 4.59 (1H, s), 4.36 (4H, m), 4.21-4.12 (12H, m), 4.07-4.00 (6H, m), 4.02 (2H, s), 3.95 (2H, s), 3.92 (2H, s), 3.52 (2H, s), 3.49 (2H, s), 1.34-1.26 (6H, m), 1.26-1.12 (26H, m). ¹³C NMR (D₆-DMSO, δ ppm): 171.10, 168.54, 168.52, 168.50, 168.48, 164.66, 164.43, 161.09, 160.99, 160.85, 160.66, 160.47, 158.68, 158.58, 147.84, 144.79, 133.35, 132.63, 131.50, 128.22, 127.09, 125.20, 124.53, 122.96, 122.88, 94.55, 94.27, 93.89, 93.79, 93.40, 88.97, 87.38, 86.48, 80.81, 65.77, 65.73, 65.39, 61.73, 61.65, 61.08, 61.04, 60.26, 60.25, 59.42, 59.34, 59.25, 57.95, 55.10, 54.95, 14.38, 14.35. MALDI TOF-MS mass: calculated (M+H⁺) 1472.56; found 1472.45.

### Example 21. Synthesis of Compound 22

A mixture of compound 13 (0.45 g, 0.50 mmol) and SnCl₂x2H₂O (1.12 g, 5.0 mmol) in abs EtOH (50 ml) was stirred for 6 h at 85° C. The mixture was evaporated to ca. half volume, H₂O (20 ml) was added. After neutralization with solid NaHCO₃, the mixture was extracted with CH₂Cl₂ (40 ml) and 25% EtOH/CH₂Cl₂ (3×20 ml) and the combined organic fraction were dried with Na₂SO₄. The product (0.38 g, 90%) was used for the next step without any further purifications. MALDI TOF-MS mass: calculated (M+H⁺) 847.55; found 847.11.

### Example 22. Synthesis of Compound 23

Compound 23 was synthesized from compound 14 using a method analogous to the synthesis described in example 21. The product (yield 40%) was purified by FC (silica gel, from 2% to 5% EtOH/CH₂Cl₂). MALDI TOF-MS mass: calculated (M+H⁺) 1019.81; found 1019.34.

### Example 23. Synthesis of Ligand Ester 24

A mixture of compound 21 (0.215 g, 0.146 mmol) and 22 (59 mg, 70 μmol) in dry TEA (1 ml) and DMF (2 ml) was de-aerated with argon. After an addition of bis(triphenylphosphine)palladium(II) chloride (10 mg, 14 μmol) and CuI (6 mg, 28 μmol), the mixture was stirred 24 h at RT. After evaporation to dryness, the product (101 mg, 40%) was purified by FC (silica gel, from 5% to 10% EtOH/CH₂Cl₂). MALDI TOF-MS mass: calculated (M+H⁺) 3536.40; found 3536.79.

### Example 24. Synthesis of Ligand Ester 25

Compound 25 was synthesized from compounds 23 and 21 using a method analogous to the synthesis described in example 23 stirring for 22 h at 55° C. The product (yield 49%) was purified by FC (silica gel, 5:89:1 EtOH/CH₂Cl₂/TEA). MALDI TOF-MS mass: calculated (M+H⁺) 3706.53; found 3706.67.

### Example 25. Synthesis of Eu Chelate 26

A mixture of ligand ester 24 (48 mg, 13.6 μmol), 0.5 M KOH in EtOH (10 ml) and CH₂Cl₂ (5 ml) was stirred for 2 h at RT and evaporated to ca. half volume. After an addition of H₂O (5 ml), the mixture was stirred for 30 min at RT. The rest of EtOH was evaporated and the aqueous solution was stirred overnight at RT. The pH was adjusted to 6.5 by addition of 6 M HCl. EuCl₃ (11 mg, 30 μmol) in water (0.2 ml) was added within 5 min and the pH was maintained at 6.0-6.5 with suitable additions of solid NaHCO₃. After stirring for overnight at RT, the pH was adjusted to 8.5 with 1 M NaOH. The precipitate was removed by centrifugation and the supernatant evaporated to dryness. The product was purified by HPLC. Two isomers were found. Yield: 29 mg (45%). R_{f}(HPLC): 18.2 and 20.4 min. MALDI TOF-MS mass: calculated (M+H⁺) 3273.53; found 3273.46.

### Example 26. Synthesis of Eu Chelate 27

Eu chelate 27 was synthesized from the ligand ester 25 using a method analogous to the synthesis described in example 25. The product (yield 45%) was purified by HPLC. Two isomers were found R_{f}(HPLC): 22.2 and 24.2 min. MALDI TOF-MS mass: calculated (M+H⁺) 3443.70; found 3441.79.

### Example 27. Synthesis of Eu Chelate 28

Eu chelate 28 was synthesized from the Eu chelate 26 using a method analogous to the synthesis described in example 6. The product was used for the antibody labelling without any further purifications. R_{f}(HPLC): 21.1 and 25.8 min.

### Example 28. Synthesis of Eu Chelate 29

Eu chelate 29 was synthesized from the Eu chelate 27 using a method analogous to the synthesis described in example 6. The product was used for the antibody labelling without any further purifications. R_{f}(HPLC): 25.5 min.

### Example 29. Synthesis of Compound 30

A mixture of compound 11 (1.08 g, 2.80 mmol), BrCH₂COO^{t}Bu (0.87 ml, 5.88 mmol), dry K₂CO₃ (1.55 g, 11.2 mmol) in dry MeCN (50 ml) was stirred for 24 h at RT. The mixture was evaporated to dryness and the product (1.54 g, 90%) was purified by FC (silica gel, from CH₂Cl₂ to 2% MeOH/CH₂Cl₂). MALDI TOF-MS mass: calculated (M+H⁺) 615.73; found 615.12.

### Example 30. Synthesis of Compound 31

A mixture of compound 31 (1.52 g, 2.47 mmol) in trifluoroacetic acid (10 ml) was stirred for 2.5 h at RT, and evaporated to dryness. The product was precipitated by Et₂O (100 ml), filtrated, washed with Et₂O and dried in vacuum desiccator. The product as TFA salt was dissolved in 10% NaOH (30 ml), the mixture was made acid (pH ca. 2.0) with 1M HCl, and the product was separated and washed with H₂O. Yield: 1.34 g (98%). MALDI TOF-MS mass: calculated (M+H⁺) 503.22; found 502.89.

### Example 31. Synthesis of Compound 32

Compound 32 was synthesized from compound 31 and 4-jodoaniline using a method analogous to the synthesis described in example 13. The product (0.45 g, 100%) was purified by FC (silica gel, EtOAc).

### Example 32. Synthesis of Compound 33

Compound 33 was synthesized from the compound 32 using a method analogous to the synthesis described in example 2 stirring for 22 h at 80° C. The product (48%) was purified by FC (silica gel, EtOAc).

### Example 33. Synthesis of Ligand Ester 34

The ligand ester 34 was synthesized from compounds 33 and 21 using a method analogous to the synthesis described in example 23. The product (61%) was purified by FC (silica gel, from 7.5:91.5:1 to 10:89:1 EtOH/CH₂Cl₂/TEA). MALDI TOF-MS mass: calculated (M+H⁺) 3534.74; found 3534.17.

### Example 34. Synthesis of Eu Chelate 35

Eu chelate 35 was synthesized from the ligand ester 34 using a method analogous to the synthesis described in example 25. The product (yield 24%) was purified by HPLC. R_{f}(HPLC): 21.3 min.

### Example 35. Synthesis of Eu Chelate 36

Eu chelate 36 was synthesized from the Eu chelate 35 using a method analogous to the synthesis described in example 6. The product was used for the antibody labelling without any further purifications.

### Example 36. Synthesis of Compound 37

2-(Boc-oxyimino)-2-phenylacetonitrile (4.93 g, 20 mmol) was added in small partitions within 20 min into a solution of diethylenetriamine (1.08 ml, 10 mmol), H₂O (10 ml), 1,4-dioxane (10 ml) and TEA (4.18 ml, 30 mmol). After stirring for overnight at RT, H₂O (20 ml) was added and the mixture was extracted with EtOAc (40 ml+20 ml). The combined organic phases were washed with 10% NaOH (2×15 ml), H₂O (2×15 ml) and dried with Na₂SO₄. The product (2.58 g, 78%) was purified by FC (silica gel, from 5:95:0 to 20:79:1 MeOH/CH₂Cl₂/TEA). ¹H NMR (CDCl₃, δ ppm): 4.94 (2H, bs), 3.21 (4H, q, J=5.8 Hz), 2.73 (4H, t, J=5.8 Hz), 1.45 (18H, s). ¹³C NMR (CDCl₃, δ ppm): 79.14, 48.74, 40.23, 28.33.

### Example 37. Synthesis of Compound 38

Trifluoroacetic acid anhydride (11.1 ml, 80 mmol) was added within 15 min into ice-cold solution of 4-aminophenylacetic acid (3.02 g, 20 mmol) in trifluoroacetic acid (30 ml). After stirring for 15 min on ice-bath, the mixture was stirred for 2 h at RT and H₂O (50 ml) was added. A cooled mixture was filtrated and the product (3.93 g, 80%) was washed with H₂O and dried. ¹H NMR (D₆-DMSO, δ ppm): 12.33 (1H, s), 11.22 (1H, s), 7.59 (2H, d, J=8.6 Hz), 7.29 (2H, d, J=8.6 Hz), 3.57 (2H, s). ¹³C NMR (D₆-DMSO, δ ppm): 171.50, 155.23, 154.94, 154.64, 154.35, 135.16, 132.86, 130.30, 121.39, 119.63, 117.34, 115.04, 112.75, 40.43.

### Example 38. Synthesis of Compound 39

N-hydroxysuccinimide (0.575 g, 5.0 mmol) and N,N-dicyclohexylcarbodiimide (1.03 g, 5.0 mmol) was added to a solution of compound 38 (1.23 g, 5.0 mmol) in dry 1,4-dioxane (20 ml). After stirring for 4 h at RT, the mixture was filtrated and the solid material was washed with 1.4-dioxane (3×5 ml) and the filtrate was evaporated to dryness. The residue was dissolved in dry DMF (15 ml) and the 6-aminohexanoic acid (0.655 g, 5.0 mmol) was added. The mixture was stirred for one week at RT. The mixture was evaporated to dryness, and after an addition of H₂O (25 ml), the cooled mixture was filtrated, the product (1.80 g, 100%) was washed with H₂O (3×10 ml) and dried. ¹H NMR (D₆-DMSO, δ ppm): 11.80 (1H, bs), 11.23 (1H, bs), 7.99 (1H, t, J=5.5 Hz), 7.57 (2H, d, J=8.6 Hz), 7.27 (2H, d, J=8.6 Hz), 3.38 (2H, s), 3.02 (2H, q, J=6.9 Hz), 2.18 (2H, t, J=7.4 Hz), 1.53-1.43 (2H, m), 1.43-1.33 (2H, m), 1.30-1.20 (2H, m). ¹³C NMR (D₆-DMSO, δ ppm): 174.79, 170.09, 155.19, 154.90, 154.61, 154.32, 134.90, 134.43, 129.80, 121.39, 117.35, 115.05, 112.75, 42.19, 38.84, 33.97, 29.16, 26.31, 24.56. MALDI TOF-MS mass: calculated (M+H⁺) 361.34; found 363.96.

### Example 39. Synthesis of Compound 40

N-hydroxysuccinimide (0.12 g, 1.0 mmol) and N,N-dicyclohexylcarbodiimide (0.21 g, 1.0 mmol) was added to a solution of compound 39 (0.36 g, 1.9 mmol) in dry 1,4-dioxane (30 ml) and DMF (5 ml). After stirring for 3 h at RT, a solution of compound 37 (0.30 g, 1.0 mmol) in 1.4-dioxane (3 ml) was added and the mixture was stirred for 2 days at RT. After evaporation to dryness, the residue was dissolved in 1,4-dioxane (10 ml), filtrated and the solid material was washed with 1,4-dioxane and the filtrate was evaporated to dryness. The residue was dissolved in CH₂Cl₂ (30 ml), washed with H₂O (3×10 ml) and dried with Na₂SO₄. The product (0.37 g, 57%) was purified with FC (silica gel, from 5% to 7.5% MeOH/CH₂Cl₂. ¹H NMR (D₆-DMSO, δ ppm): 11.21 (1H, s), 7.80 (1H, t, J=5.5 Hz), 7.57 (2H, d, J=8.5 Hz), 7.27 (2H, t, J=8.5 Hz), 6.98 (1H, t, J=5.9 Hz), 6.80 (1H, t, J=5.4 Hz), 3.58 (2H, s), 3.27 (2H, t, J=6.5 Hz), 3.23 (2H, t, J=6.5 Hz), 3.05-2.97 (4H, m), 2.23 (2H, t, J=7.3 Hz), 1.50-1.44 (2H, m), 1.44-1.34 (2H, m), 1.36 (18H, s), 1.28-1.21 (2H, m). ¹³C NMR (D₆-DMSO, δ ppm): 172.70, 170.15, 156.13, 156.08, 155.21, 154.96, 154.72, 154.47, 135.00, 134.51, 129.91, 121.46, 119.16, 117.25, 115.34, 113.43, 78.25, 78.03, 55.38, 47.72, 45.64, 42.28, 39.03, 32.44, 29.47, 28.69, 28.65, 26.67, 25.08. MALDI TOF-MS mass: calculated (M+H⁺) 646.34; found 647.36.

Beside of the wanted product 40, 0.18 g of NHS-activated form of 39 was obtained from which an additional amount of the compound 40 (0.164 g, 65%) was prepared in DMF and TEA.

### Example 40. Synthesis of Compound 41

A mixture of compound 40 (0.52 g, 0.812 mmol) in CH₂Cl₂ (20 ml) and trifluoroacetic acid (5 ml) was stirred for 4.5 h at RT, and evaporated to dryness. After an addition of Et₂O (30 ml), the product (0.532 g, 97%) was removed by centrifugation, washed with Et₂O (2×15 ml) was dried in vacuum desiccator. ¹H NMR (D₆-DMSO, δ ppm): 11.25 (1H, s), 8.06 (1H, t, J=5.5 Hz), 7.99 (2H, bs), 7.79 (2H, bs), 7.57 (2H, d, J=8.5 Hz), 7.27 (2H, d, J=8.5 Hz), 3.53.3.45 (4H, m), 3.05-2.96 (4H, m), 2.96-2.90 (2H, m), 2.30 (2H, t, J=7.6 Hz), 1.53-1.45 (2H, m), 1.45-1.37 (2H, m), 1.31-1.25 (2H, m). ¹³C NMR (D₆-DMSO, δ ppm): 174.01, 170.22, 159.17, 158.95, 158.72, 158.50, 155.25, 155.00, 154.76, 154.52, 135.02, 134.50, 129.92, 121.50, 118.17, 117.90, 117.25, 115.94, 115.33, 113.99, 44.99, 43.13, 42.27, 39.03, 37.58, 37.36, 32.48, 29.50, 26.53, 24.68. MALDI TOF-MS mass: calculated (M+2H⁺) 447.50; found 447.16.

### Example 41. Synthesis of Compound 42

A mixture of compound 1 (180 mg, 0.40 mmol), 41 (67 mg, 0.10 mol) and DIPEA (209 μl, 1.2 mmol) in dry MeCN (5 ml) was stirred overnight at 52° C. After evaporation to dryness, the product (163 mg, 84%) was purified by FC (silica gel, 5% MeOH/CH₂Cl₂). MALDI TOF-MS mass: calculated (M+H⁺) 1932.45, 1930.45; found 1932.59, 1930.69.

### Example 42. Synthesis of Ligand Ester 43

A mixture of compound 42 (160 mg, 84.0 μmol) and diethyl 2,2′-{{4-[(trimethylsilyl)ethynyl]-1,3-phenylene}bis(oxy)}diacetate (129 mg, 0.422 mmol; Sund, H., et al. Molecules 22(2017)1807) in dry TEA (1 ml) and THF (2 ml) was de-aerated with argon. After an addition of bis(triphenylphosphine)-palladium(II) chloride (10 mg, 14 μmol) and CuI (6 mg, 28 μmol), the mixture was stirred 24 h at 55° C. After evaporation to dryness, the product (148 mg, 62%) was purified by FC (silica gel, from 5% to 10% EtOH/CH₂Cl₂). MALDI TOF-MS mass: calculated (M+H⁺) 2832.19; found 2831.19.

### Example 43. Synthesis of Ligand Ester 44

This ligand ester 44 was synthesized from compound 42 and ethyl 2-(4-ethynyl-3-methoxyphenoxy)acetate using a method analogous to the synthesis described in example 41. The product (60%) was purified by FC (silica gel, from 5% to 10% EtOH/CH₂Cl₂). MALDI TOF-MS mass: calculated (M+2H⁺) 2545.12; found 2545.17.

### Example 44. Synthesis of Eu Chelate 45

Eu chelate 45 was synthesized from the ligand ester 43 using a method analogous to the synthesis described in example 25. The product (46%) was purified by HPLC. R_{f}(HPLC): 12.8 min. UV: 348 nm.

### Example 45. Synthesis of Eu Chelate 46

Eu chelate 46 was synthesized from the ligand ester 44 using a method analogous to the synthesis described in example 25. The product (yield 73%) was purified by HPLC. R_{f}(HPLC): 16.2 min. UV: 348 nm.

### Example 46. Synthesis of Eu Chelate 47

Eu chelate 47 was synthesized from the Eu chelate 45 using a method analogous to the synthesis described in example 6. The product was used for the antibody labelling without any further purifications.

### Example 47. Synthesis of Eu Chelate 48

Eu chelate 48 was synthesized from the Eu chelate 46 using a method analogous to the synthesis described in example 6. The product was used for the antibody labelling without any further purifications.

### Example 48. Synthesis of Compound 49

A mixture of Boc-Gly-OSu (0.27 g, 1.0 mmol) and 4-iodoaniline (0.22 g, 1.0 mmol) in dry DMF (2.0 ml) was stirred for one week at RT. After evaporation to dryness, the product (0.32 g, 85%) was purified by FC (silica gel, 50% EtOAc/petroleum ether). ¹H NMR (D₆-DMSO, δ ppm): 10.3 (1H, s), 7.64 (2H, d, J=8.8 Hz), 7.43 (2H, d, J=8.8 Hz), 7.06 (1H, t, J=6.1 Hz), 3.70 (2H, d, J=6.1 HZ), 1.39 (9H, s). ¹³C NMR (D₆-DMSO, δ ppm): 168.91, 156.40, 139.26, 137.86, 121.71, 87.04, 78.54, 44.29, 28.67. MALDI TOF-MS mass: calculated (M+2H⁺) 378.05; found 378.71.

### Example 49. Synthesis of Compound 50

A mixture of compound 49 (0.65 g, 1.73 mmol) and TFA (10 ml) was stirred for 5 h at RT and was evaporated to dryness. After and addition of Et₂O (50 ml), the mixture was stirred for ca. 30 min, the product (0.63 g, 93%) was filtered, washed with Et₂O (50 ml) and dried in in vacuum desiccator. ¹H NMR (D₆-DMSO, δ ppm): 10.59 (1H, s), 8.15 (2H, s), 7.70 (2H, d, J=8.8 Hz), 7.35 (2H, d, J=8.8 Hz), 3.79 (2H, s). ¹³C NMR (D₆-DMSO, δ ppm): 165.49, 158.79, 158.58, 158.38, 158.17, 138.48, 138.16, 121.76, 120.75, 118.77, 116.78, 88.08, 41.57, 31.18. MALDI TOF-MS mass: calculated (M+H⁺) 276.99; found 276.53.

### Example 50. Synthesis of Compound 51

A mixture of compound 50 (0.62 g, 1.59 mmol) and DIPEA (1.38 ml, 7.95 mmol) in dry MeCN (20 ml) was stirred at RT until a clear mixture was obtained and BrCH₂COOEt (176 μl, 1.59 mmol) was added. After stirring overnight at 55° C., the mixture was evaporated to dryness and the product (1.12 g, 67%) was purified by FC (silica gel, from 50:49:1 to 75:24:1 EtOAc/petroleum ether/TEA). ¹H NMR (D₆-DMSO, δ ppm): 9.98 (1H, s), 7.63 (2H, d, J=8.8 Hz), 7.47 (2H, d, J=8.8 Hz), 4.11 (2H, q, J=7.1 Hz), 3.42 (2H, s), 1.19 (3H, t, J=7.1 Hz). ¹³C NMR (D₆-DMSO, δ ppm): 172.63, 170.65, 139.02, 137.82, 121.82, 87.17, 60.55, 52.66, 50.48, 14.60. MALDI TOF-MS mass: calculated (M+H⁺) 363.02; found 362.63.

### Example 51. Synthesis of Compound 53

A mixture of compound 52 (0.43 g, 0.90 mmol; Sund, H., et al. Molecules 22(2017)₁₈₀₇), NH₂CH₂COOEt x HCl (31 mg, 0.22 mmol), DIPEA (160 μl, 0.90 mmol) in dry MeCN (15 ml) was stirred for 2 h at RT. After evaporation to dryness, the product (130 mg, 66%) was purified by FC (silica gel, 2% MeOH/CH₂Cl₂).

### Example 52. Synthesis of Compound 54

A mixture of compound 51 (151 mg, 0.20 mmol), 53 (89 mg, 0.10 mmol) and dry K₂CO₃ (55 mg, 0.40 mmol) in dry MeCN (5 ml) was stirred for 3-4 days at RT. The mixture was filtrated, the precipitate was washed with MeCN and the filtrate was evaporated to dryness. The product (101 mg, 69%) was purified by FC (silica gel, from 2 to 5% MeOH/CH₂Cl₂). MALDI TOF-MS mass: calculated (M+H⁺) 1456.23; found 1456.35.

### Example 53. Synthesis of Ligand Ester 55

A mixture of compound 21 (255 mg, 173 μmol) and 54 (101 mg, 70 μmol) in dry TEA (1 ml) and DMF (2 ml) was de-aerated with argon. After an addition of bis(triphenylphosphine)palladium(II) chloride (10 mg, 14 μmol) and CuI (6 mg, 28 μmol), the mixture was stirred 24 h at RT. After evaporation to dryness, the product (193 mg, 67%) was purified by FC (silica gel, from 5% to 10% EtOH/CH₂Cl₂). MALDI TOF-MS mass: calculated (M+Na⁺) 4166.50; found 4166.02.

### Example 54. Synthesis of Eu Chelate 56

Eu chelate 56 was synthesized from the ligand ester 55 using a method analogous to the synthesis described in example 25. The product (yield 18%) was purified by HPLC. R_{f}(HPLC): 14.3 min. MALDI TOF-MS mass: calculated (M+H⁺) 3756.52; found 3756.51.

### Example 55. Synthesis of Eu Chelate 57

Eu chelate 57 was synthesized from the Eu chelate 56 using a method analogous to the synthesis described in example 6. The product was used for the antibody labelling without any further purifications.

### Example 56. Synthesis of Eu Chelate 58

Eu chelate 58 was synthesized from ethyl 2-{{2-{{2-[2-(4-iodophenoxy)acetamido]ethyl}amino}-2-oxoethyl}amino}acetate and 53 using methods analogous to the synthesis steps described for the Eu chelate 56 in examples from 52 to 55.

### Example 57. Synthesis of Eu Chelate 59

This Eu chelate 59 was synthesized from the Eu chelate 58 using a method analogous to the synthesis described in example 6. The product was used for the antibody labelling without any further purifications.

### Example 58. Synthesis of Compound 60

A mixture of compound 52 (0.24 g, 0.50 mmol; Sund, H., et al. Molecules 22(2017)1807), NH(CH₂COOEt)₂ (95 mg, 0.50 mmol), dry K₂CO₃ (0.35 g, 2.5 mmol) in dry MeCN (10 ml) under argon was stirred for 5.5 h at RT. After filtration and evaporation to dryness, the product (97 mg, 33%) was purified by FC (silica gel, 30% EtOAc/petroleum ether). The product was used directly in the next step as it does not tolerate storage.

### Example 59. Synthesis of Compound 61

A mixture of compound 60 (90 mg, 0.154 mmol), BocNH(CH₂)₆NH₂ (0.17 g, 0.77 mmol), dry K₂CO₃ (0.21 g, 1.54 mmol) in dry MeCN (5 ml) under argon was stirred for 3 h at RT. After evaporation to dryness, the product (85 mg, 77%) was purified by FC (silica gel, 10% MeOH/CH₂Cl₂). MALDI TOF-MS mass: calculated (M+H⁺) 720.36; found 720.43.

### Example 60. Synthesis of Compound 62

A mixture of compound 61 (106 mg, 0.15 mmol), compound 1 (68 g, 0.15 mmol), dry K₂CO₃ (41 mg, 0.30 mmol) in dry MeCN (5 ml) under argon was stirred for 7 h at RT. After filtration and evaporation to dryness, the product (97 mg, 60%) was purified by FC (silica gel, 5% MeOH/CH₂Cl₂). MALDI TOF-MS mass: calculated (M+H⁺) 1092.41, 1090.41; found 1092.00, 1089.95.

### Example 61. Synthesis of Compound 63

A solution of compound 62 (97 mg, 89 μmol) in trifluoroacetic acid (1.3 ml) was stirred for 2 h at RT. The solution was evaporated to dryness and further evaporated twice from diethyl ether (2×20 ml). The residue was dissolved in dry MeCN (3 ml), and after an addition of DIPEA (0.32 ml, 1.8 mmol) and compound 1 (81 mg, 180 μmol), the mixture was stirred for 4 d at RT under argon. After evaporation to dryness, the product (125 mg, 81%) was purified by FC (silica gel, 5% MeOH/CH₂Cl₂). MALDI TOF-MS mass: calculated (M+H⁺) 1734.46, 1732.46; found 1734.64, 1731.73.

### Example 62. Synthesis of Ligand Ester 64

The ligand ester 64 was synthesized from compound 63 and triethyl 2,2′,2″-{[4-(ethynyl)benzene-1,3,5-triyl]-tris(oxy)triacetate in dry DMF instead of THF using a method analogous to the synthesis described in example 4. The product (yield 33%) was purified by FC (silica gel, from 5 to 10% MeOH/CH₂Cl₂). MALDI TOF-MS mass: calculated (M+H⁺) 2716.12; found 2715.52.

### Example 63. Synthesis of Eu Chelate 65

Eu chelate 65 was synthesized from the ligand ester 64 using a method analogous to the synthesis described in example 5. The product (yield 47%) was purified by HPLC. R_{f}(HPLC): 14.1 min. UV: 347 nm.

### Example 64. Synthesis of Eu Chelate 66

Eu chelate 66 was synthesized from the Eu chelate 65 using a method analogous to the synthesis described in example 6. The product was used for the antibody labelling without any further purifications.

### Example 65. Labeling of Antibody with Labelling Reagents 7, 28, 47, 57, 59 and 66

Labeling of a TnI antibody was performed similarly as described in Sund, H., et al., et al. Molecules 22(2017)1807 by using 350 mM Na₂CO₃ buffer (pH 9.8) as reaction buffer and 300 fold excess of the labelling reagents 7, 28, 47, 57, 59 or 66. The reactions were carried out overnight at RT. The labeled antibody was separated from the excess of chelates on Superdex 200 GL 10/30 gel filtration column (GE healthcare) by using TRIS-saline-azide buffer (50 mM TRIS, 0.9% NaCl, pH 7.75) as an eluent. The fractions containing the antibody were pooled and the Eu concentration was measured by UV.

### Example 66. Troponin I Immunoassay

The TnI antibody labeled with the chelate 7, 28, 47, 57, 59 and 66 was tested in sandwich immunoassay for cardiac troponin I. As a reference compound a TnI antibody labelled with Ref 1 (Von Lode, P., et al., Anal. Chem. 74(2003)3193), Ref 2 (Sund, H., et al., Molecules 22(2017)₁₈₀) and Ref 3 in FIG. 10 was used. 10 μl of diluted tracer antibody (3 ng/μl or 5 ng/μl) and 35 μl of TnI standard solution were pipetted to a pre-coated assay well (single wells in 96 well plate format, wells coated with streptavidin and a biotinylated capture antibody against TnI, Radiometer Turku Oy). The reaction mixtures were incubated for 20 min at 36° C. with shaking. The wells were washed 6 times and dried prior to measurement with Victor™ Plate fluorometer. The brightness of the novel labeling reagents were estimated from the measured signals of novel chelates and Ref 1-3, and by using the brightness values of Ref 1-3 which were 8000, 37200 M⁻¹ cm⁻¹ (Sund, H., et al. Molecules 22(2017)180) and 22800 M⁻¹ cm⁻¹ respectively. The preliminary results are summarized in Table 1.

TABLE 1

| Chelate label | εϕ (M⁻¹cm⁻¹) |
|---|---|
| 7 | 38000 |
| 28 | 58000 |
| 47 | 44000 |
| 57 | 55000 |
| 59 | 75000 |
| 66 | 41000 |

As shown from the values of Table 1, the measured brightnesses are really high and based on the observations the labels with two binding groups seem to have unexpected high luminescence.

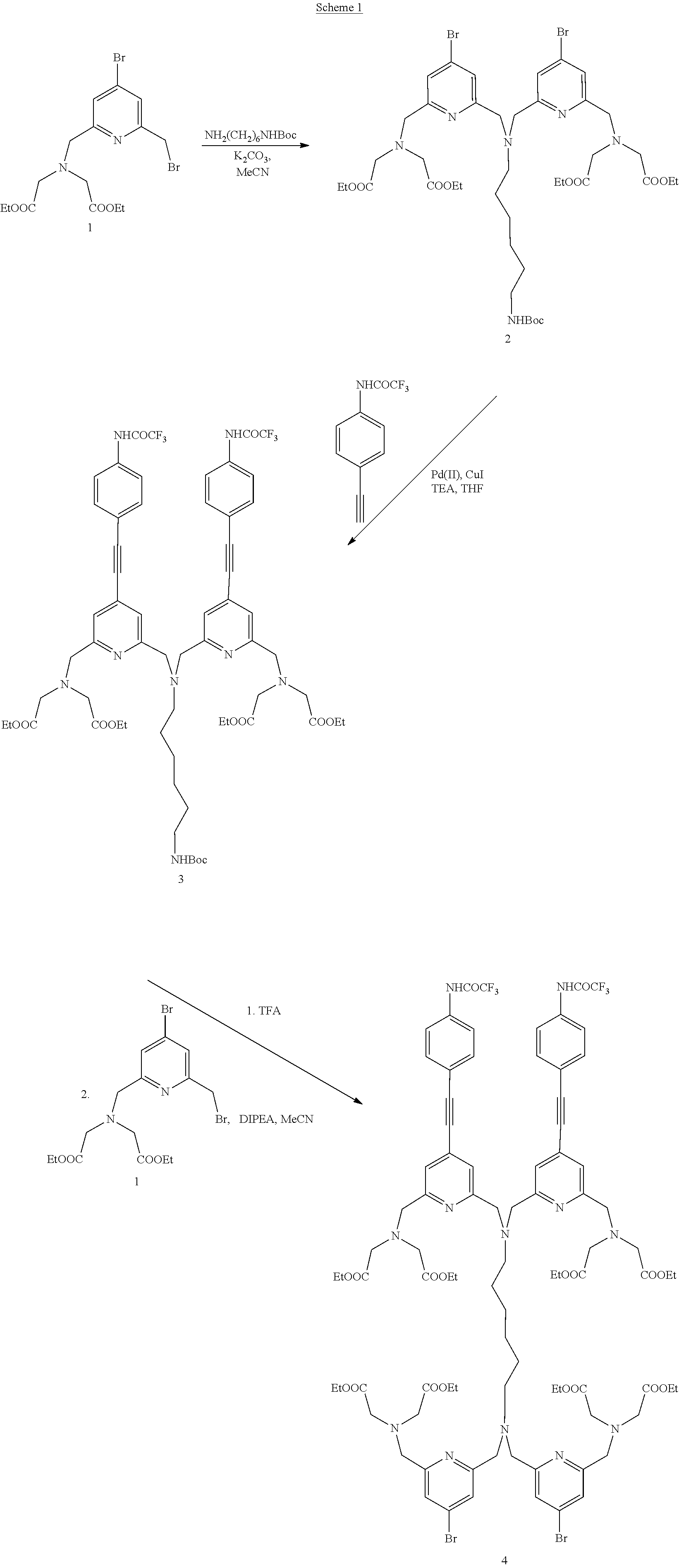

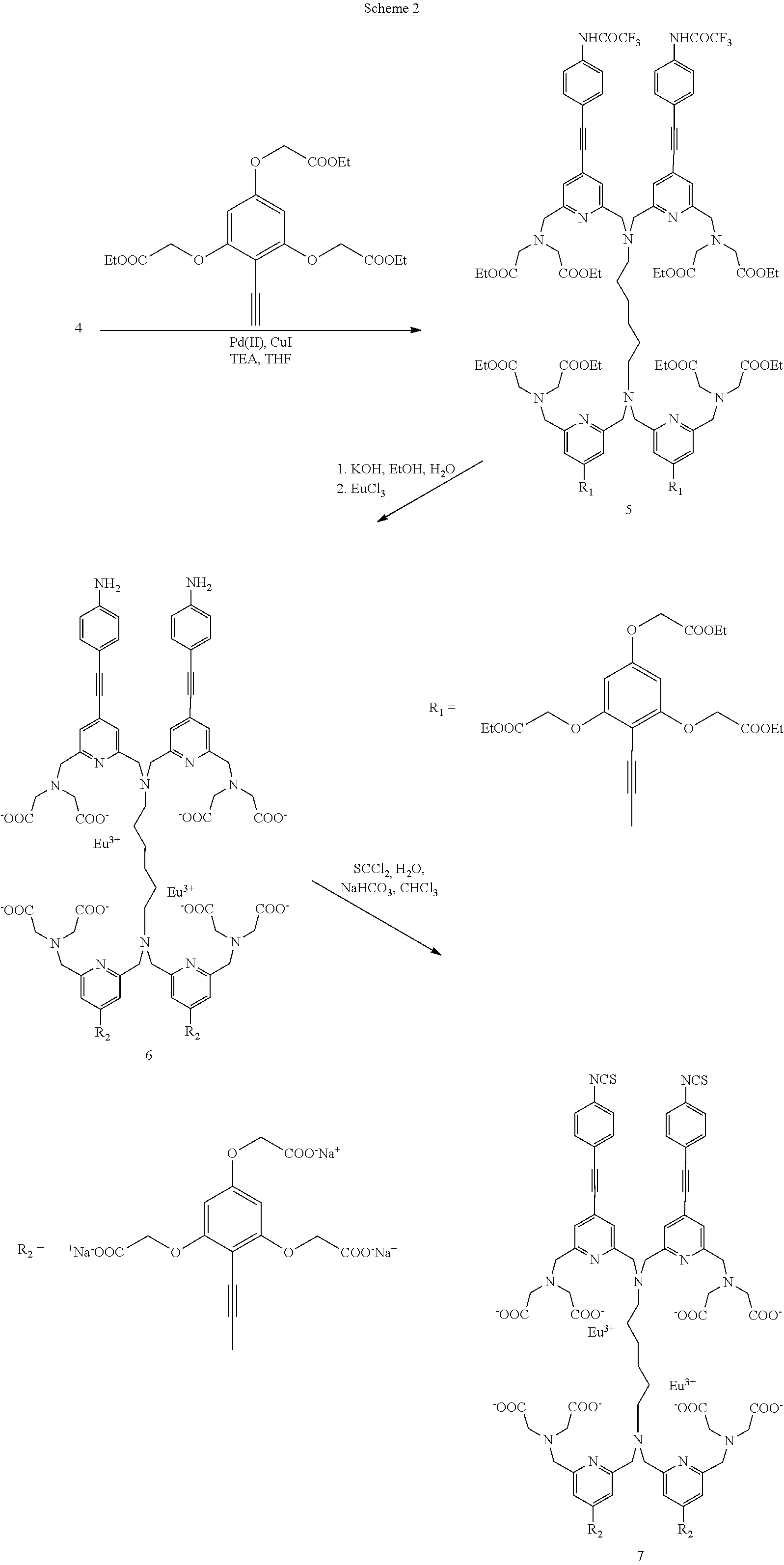

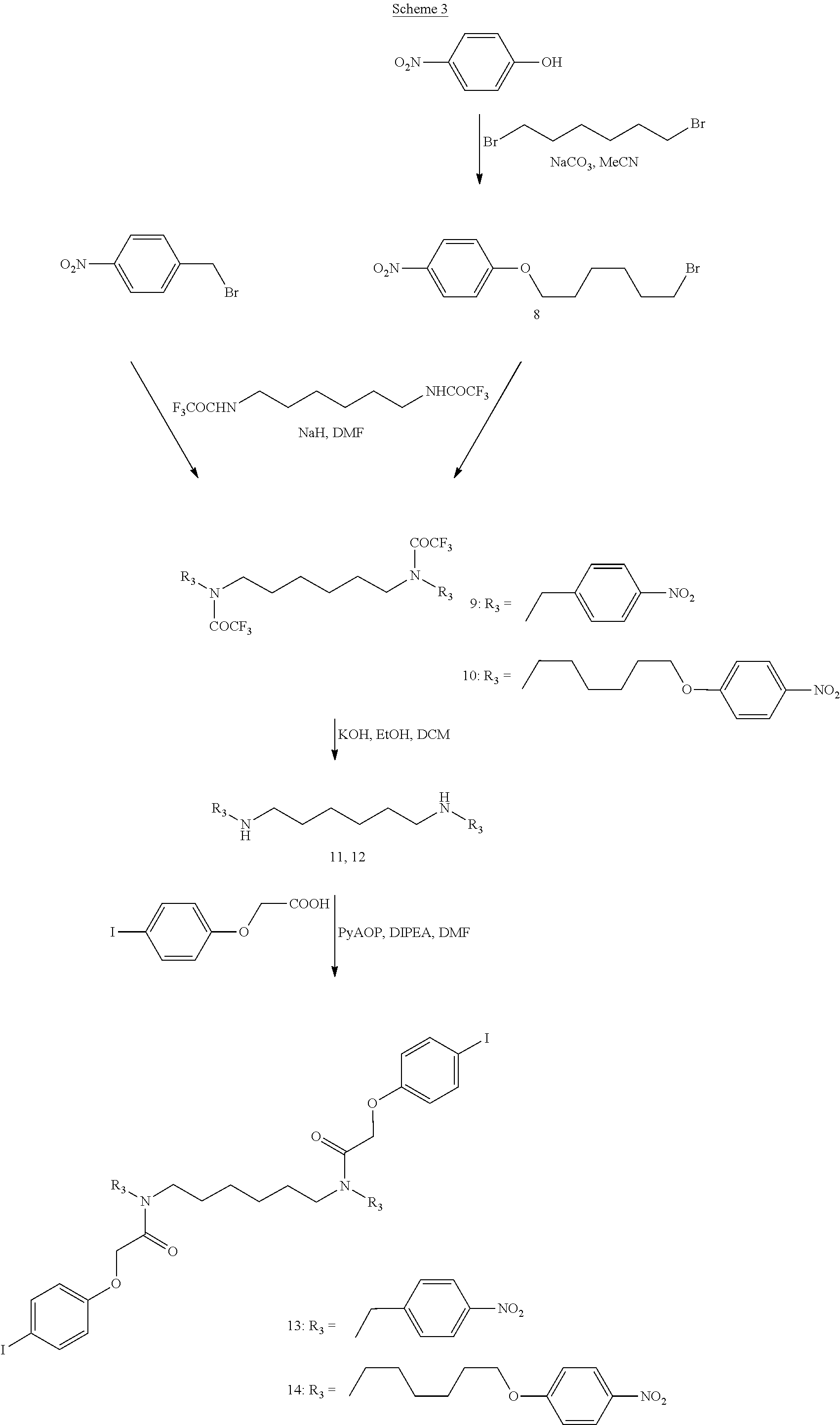

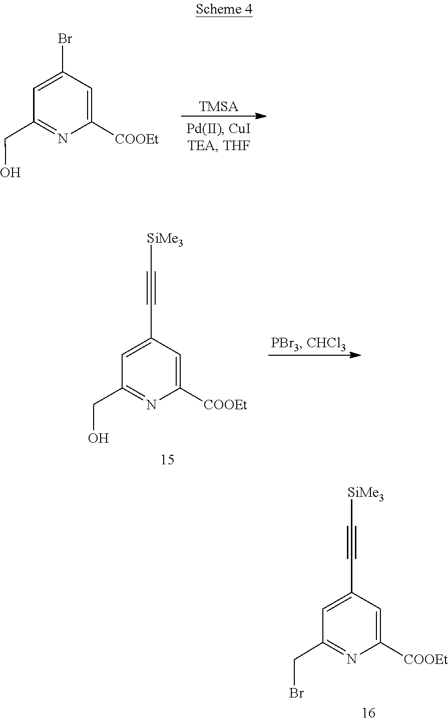

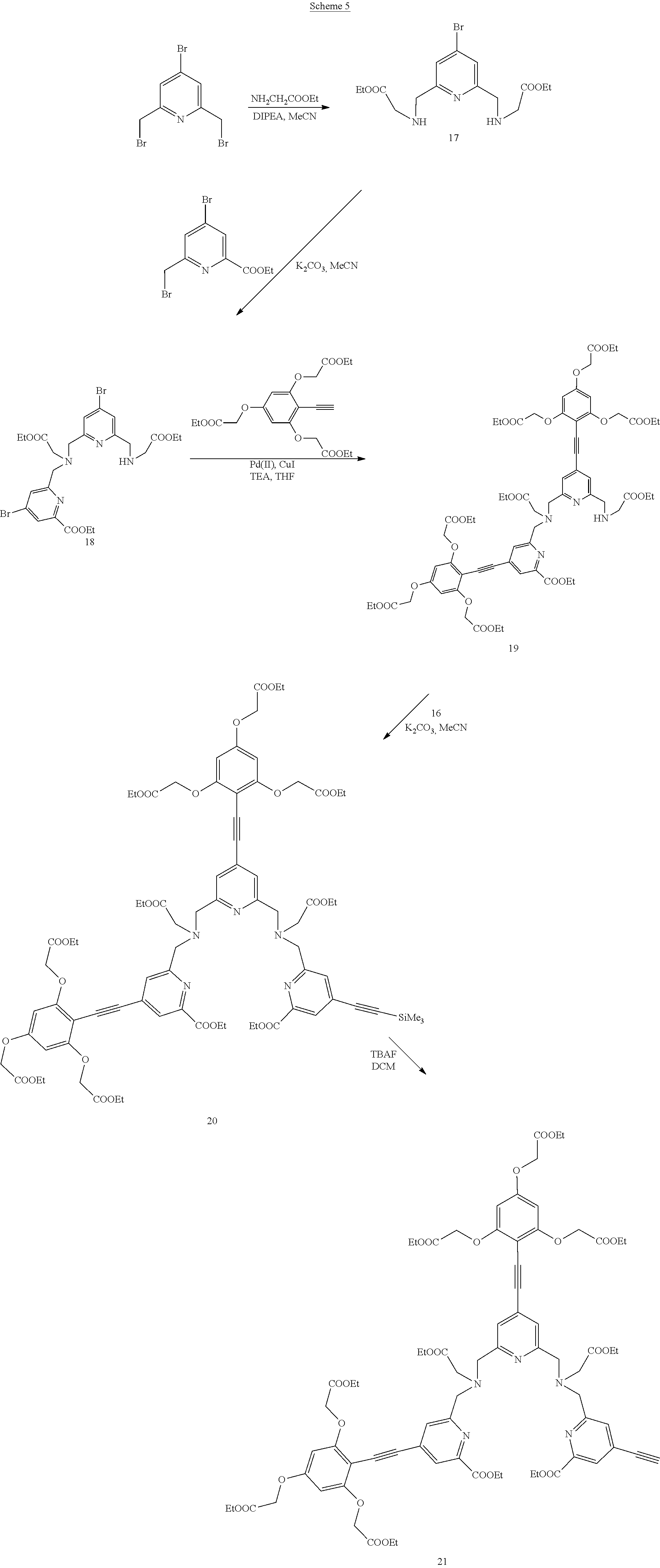

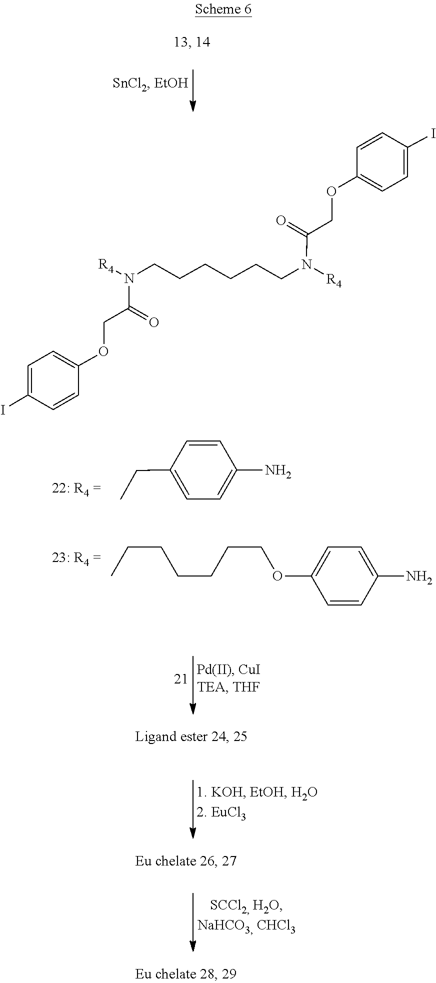

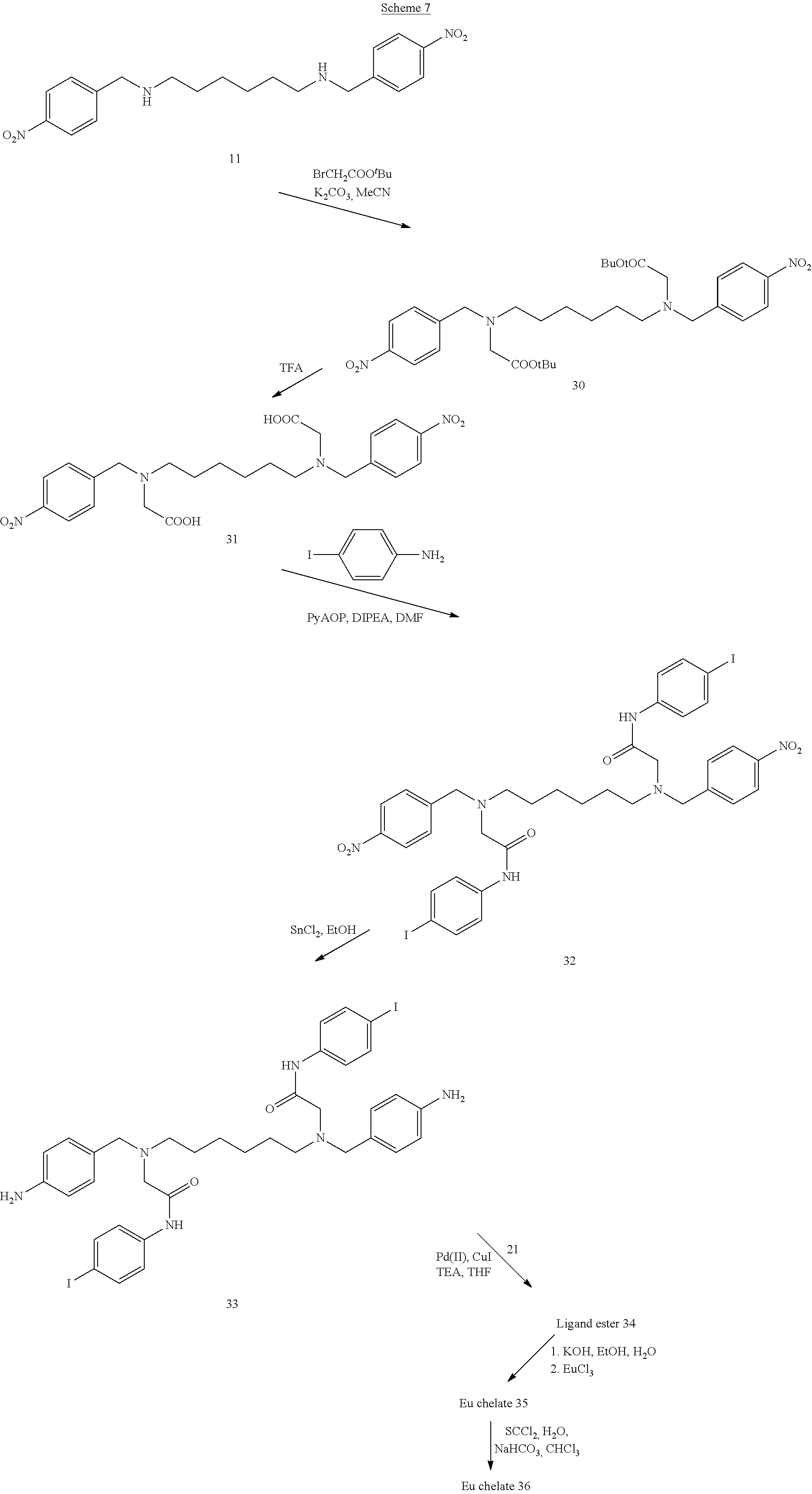

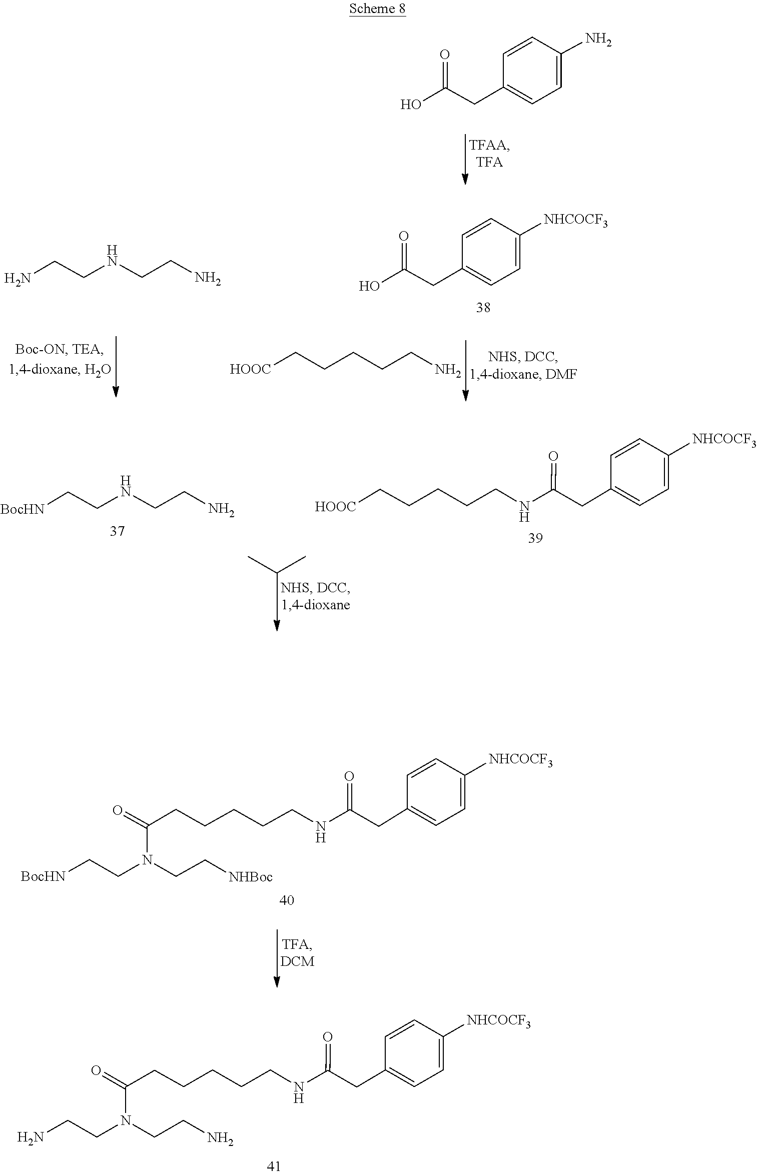

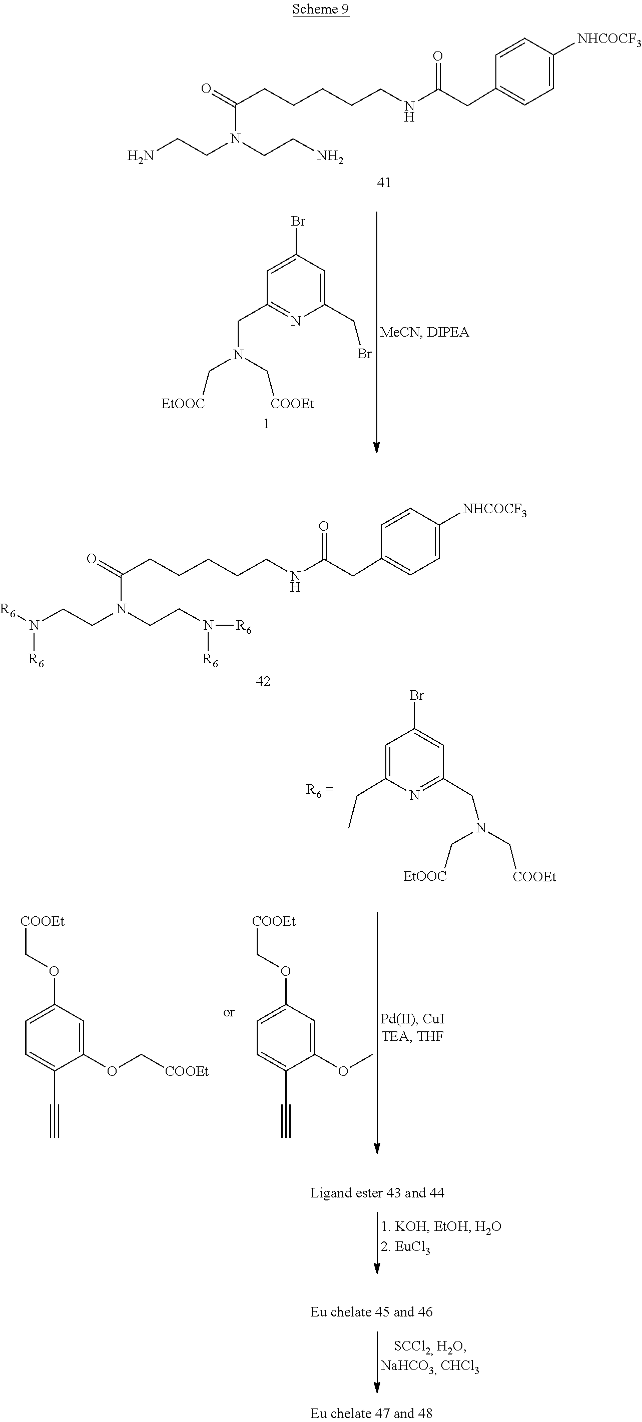

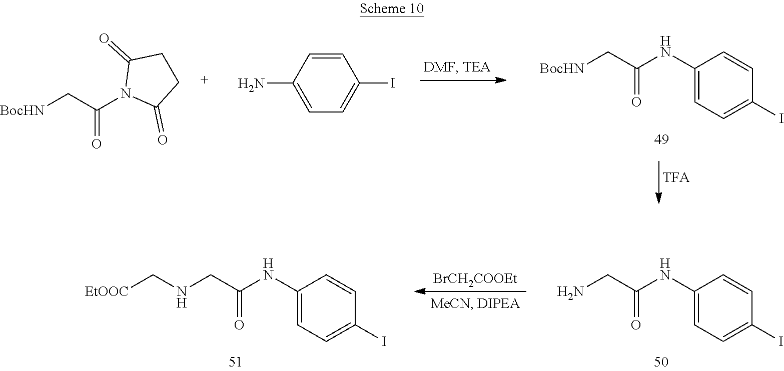 [SMILES: CC(C)(C)OC(=O)NCC(=O)N1C(=O)CCC1=O ; Nc1ccc(I)cc1 ; CC(C)(C)OC(=O)NCC(=O)Nc1ccc(I)cc1 ; CCOC(=O)CNCC(=O)Nc1ccc(I)cc1 ; NCC(=O)Nc1ccc(I)cc1 ; "BrCH2COOEt"]

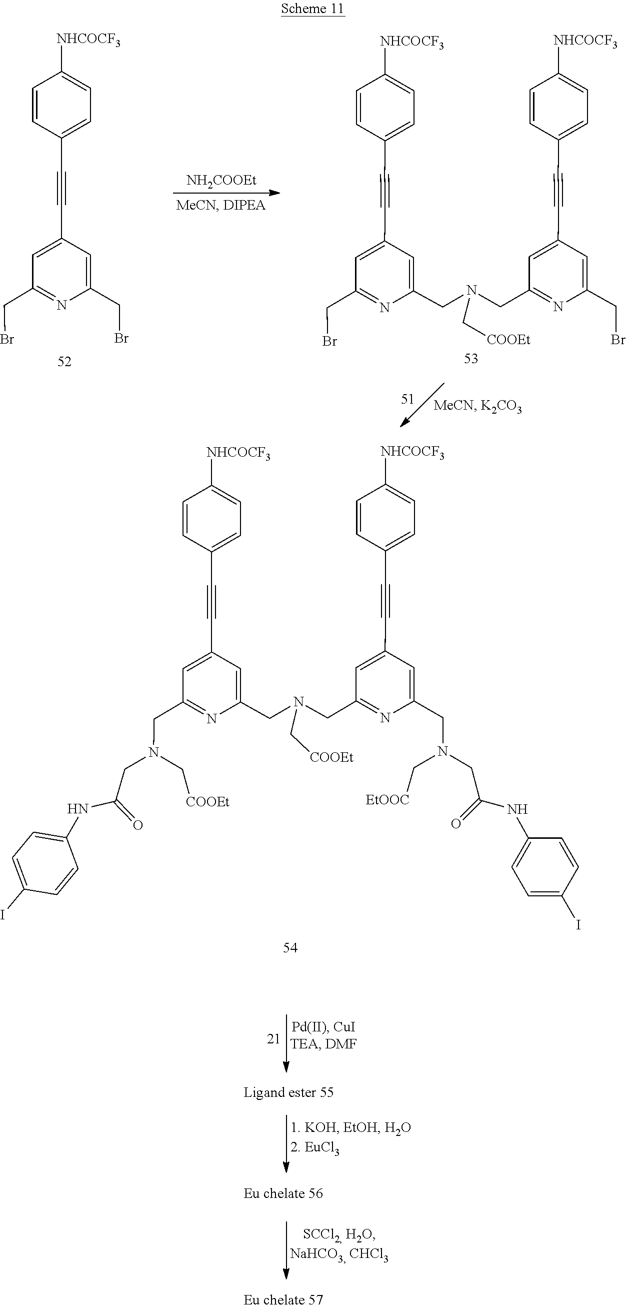

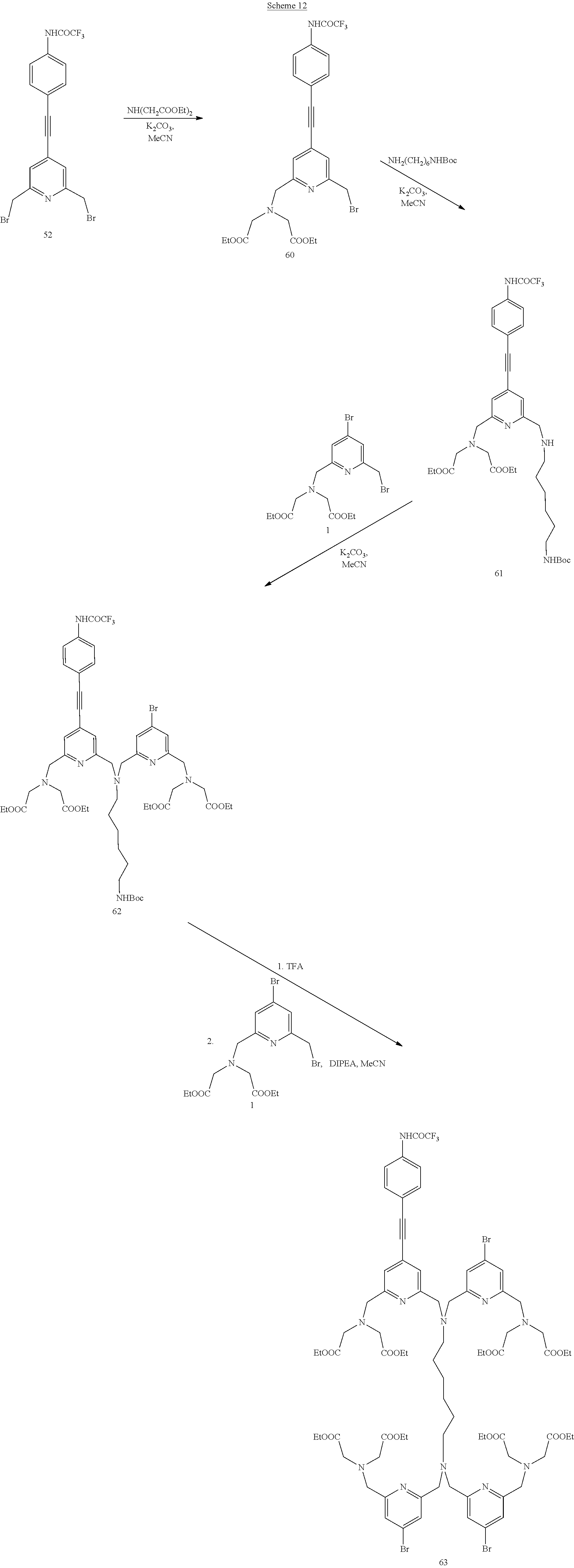

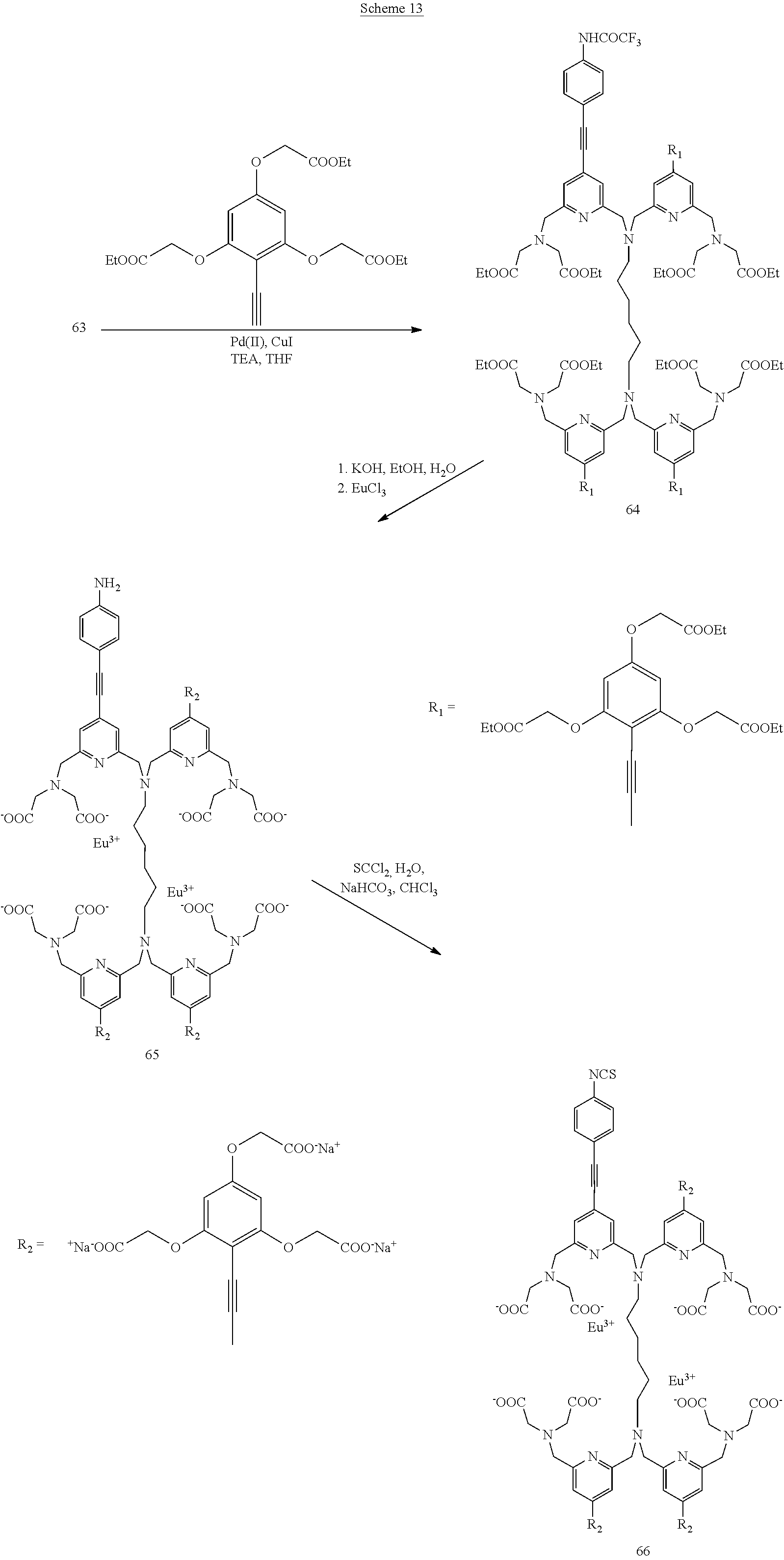

1. A compound of formula (I) 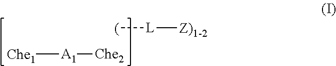
or a salt thereof, wherein
(i) the solid lines represent covalent bonds;
(ii) the dashed line represents a covalent bond of the group -L-Z to any one of the groups Che₁, A₁, and Che₂;
and wherein
L is in each case independently absent or selected from linker groups comprising from 1 to 10 moieties selected from —(CH₂)₁₋₈ —, —CH═CH—, —C≡C—, —O—, —S—, —S—S—, —C(═O)—, —C(═O)NH—, —NHC(═O)—, —C(═O)N(C₁-C₈-alkyl)-, —N(C₁-C₆-alkyl)C(═O)—, —NHC(═S)NH—, —CH[(CH₂)₀₋₆C(═O)O⁻]—, —CH[(CH₂)₀₋₆C(═O)OH]—, and biradicals of 5- to 10-membered aromatic or heteroaromatic monocyclic or bicyclic rings, wherein the heteroaromatic ring contains one or more, same or different heteroatoms N, O, or S;
Z is in each case independently selected from reactive groups selected from —N₃, —C≡CH, —CH═CH₂, —NH₂, —O—NH₂, —C(═O)OH, —CH(═O), —SH, —OH, maleimido and activated derivatives thereof including —NCO, —NCS, —N⁺≡N, bromoacetamido, iodoacetamido, reactive esters, pyridyl-2-dithio, and 6-substituted 4-chloro-1,3,5-triazin-2-ylamino and 4-chloro-1,3,5-triazin-2-yloxy;
wherein the substituent in the 6-position of the 4-chloro-1,3,5-triazin-2-ylamino or 4-chloro-1,3,5-triazin-2-yloxy is selected from —H, -halogen, —SH, —NH₂, —C₁-C₆-alkyl, —O(C₁-C₆-alkyl), —OAryl, —S(C₁-C₆-alkyl), —SAryl, —N(C₁-C₆-alkyl)₂, and N(Aryl)₂;
wherein the carbon atoms of the aforementioned groups are unsubstituted or substituted by one or more substituents selected from —CN, -halogen, —SH, —C(═O)H, —C(═O)OH, C₁-C₆-alkyl, C₁-C₆-haloalkyl, —O(C₁-C₆-alkyl), —C(═O)(C₁-C₆-alkyl), —C(═O)O(C₁-C₆-alkyl), and phenyl;
A₁ is a bridging group comprising from one to three separate straight or branched, saturated or unsaturated carbon-based chains including from 1 to 12 carbon atoms, wherein the carbon-based chains are free of or comprise one to ten, same or different groups selected from —O—, —S—, —NH—, —NR₁—, —C(═O)NH—, —NHC(═O)—, —C(═O)NR₁—, —NR₁C(═O)— and —C(═O)—,
or
A₁ a bridging chelate moiety -Che₃- of the following general formula 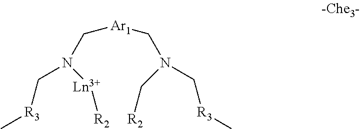
and wherein
Che₁ and Che₂ are independently selected from the chelate moieties Che I, Che II, Che III, Che IV, Che V, Che VI, and Che VII of the following general formulae: 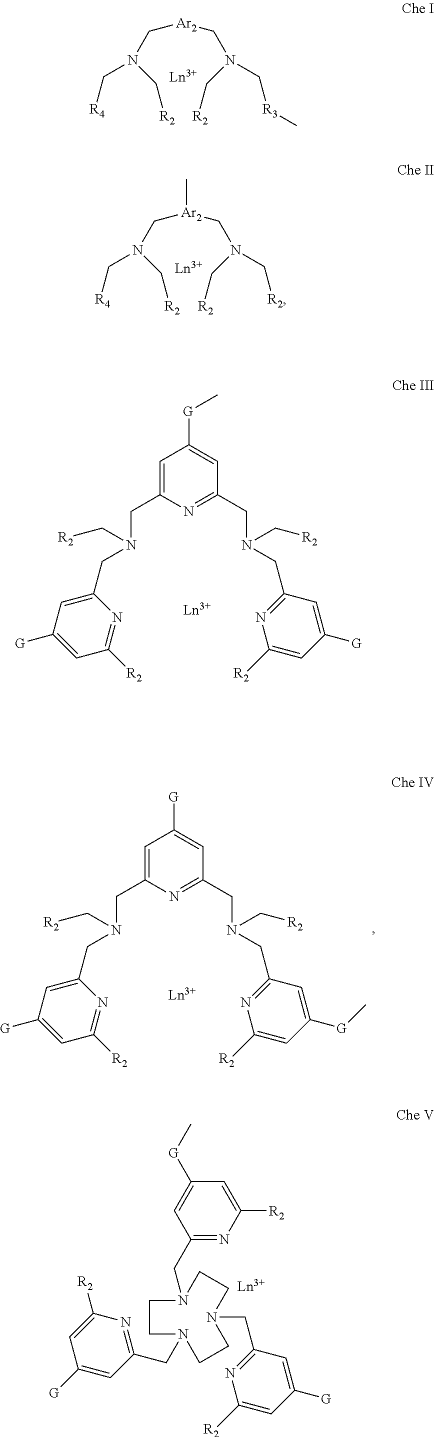 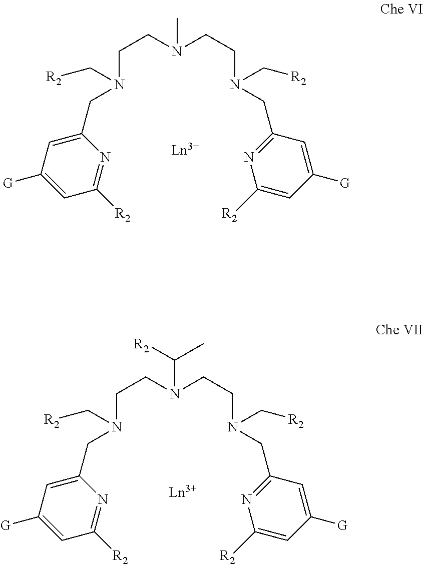
and wherein
R₁ is in each case independently selected from C₁-C₆-alkyl, and from the option of representing one of the one or two groups -L-Z;
R₂ is in each case independently selected from —C(═O)O⁻, —P(═O)O₂²⁻, P(═O)MeO⁻, —P(═O)PhO⁻, and the C₁-C₆-alkyl esters thereof, and from the option of representing one of the one or two groups -L-Z;
R₃ is in each case independently selected from the bridging groups
—C(═O)NH—, —C(═O)NH—(C₁-C₆-alkylene)-NHC(═O)—, —C(═O)NH—(C₁-C₆-alkylene)-O—, —C(═O)NH—(C₁-C₆-alkylene)-C(═O)NH—,
—C(═O)NH—(C₁-C₆-alkylene)-S—, —C(═O)NH—(C₁-C₆-alkylene)-NHC(═O)—(C₁-C₆-alkylene)-NHC(═O)—,
—C(═O)NH—(C₁-C₆-alkylene)-NHC(═O)—(C₁-C₆-alkylene)-O—, —C(═O)NH—(C₁-C₆-alkylene)-NHC(═O)—(C₁-C₆-alkylene)-C(═O)NH—, —C(═O)NH—(C₁-C₆-alkylene)-NHC(═O)—(C₁-C₆-alkylene)-S—, —C(═O)NH—(C₁-C₆-alkylene)-O—(C₁-C₆-alkylene)-NHC(═O)—,
—C(═O)NH—(C₁-C₆-alkylene)-O—(C₁-C₆-alkylene)-O—, —C(═O)NH—(C₁-C₆-alkylene)-O—(C₁-C₆-alkylene)-S—,
—C(═O)NH—(C₁-C₆-alkylene)-O—(C₁-C₆-alkylene)-C(═O)NH—, —C(═O)NH—(C₁-C₆-alkylene)-C(═O)NH—(C₁-C₆-alkylene)-NHC(═O)—, —C(═O)NH—(C₁-C₆-alkylene)-C(═O)NH—(C₁-C₆-alkylene)-O—, —C(═O)NH—(C₁-C₆-alkylene)-C(═O)NH—(C₁-C₆-alkylene)-S—, —C(═O)NH—(C₁-C₆-alkylene)-C(═O)NH—(C₁-C₆-alkylene)-C(═O)NH—, and —P(═O)O₂²⁻;
R₄ is in each case independently selected from
—CH₂N(CH₂C(═O)O⁻)₂, —CH₂N(CH₂P(═O)O₂²⁻)₂, —CH₂N(CH₂P(═O)MeO⁻)₂, —CH₂N(CH₂P(═O)PhO⁻)₂, and from the options defined for R₂;
Ln³⁺ is in each case independently selected from the lanthanide ions Eu³⁺, Tb³⁺, Sm³⁺ and Dy³⁺, wherein the lanthanide ion forms from seven to ten coordination bonds with the heteroatoms oxygen and nitrogen in the chelate moieties Che₁, Che₂, and Che₃ to form from two to three separate internal chelate moieties;
Ar₁ is selected from the following groups 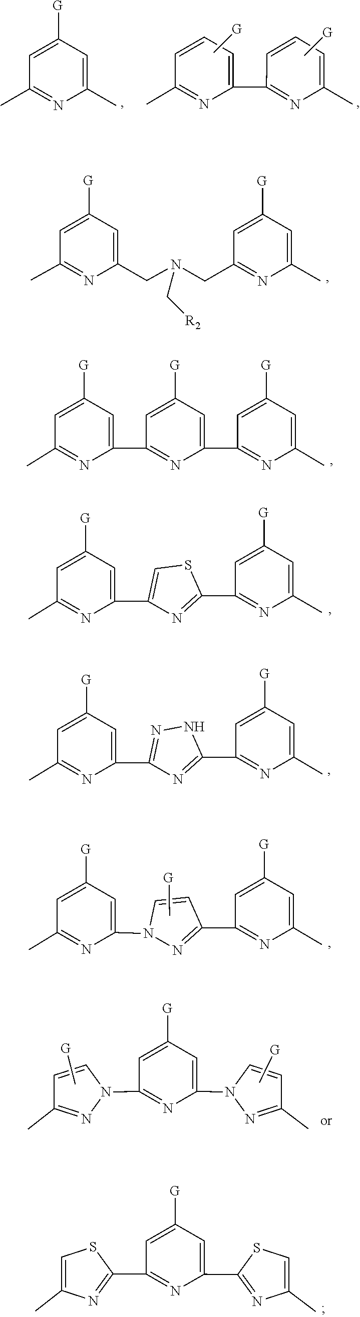
Ar₂ is in each case independently selected from the following groups 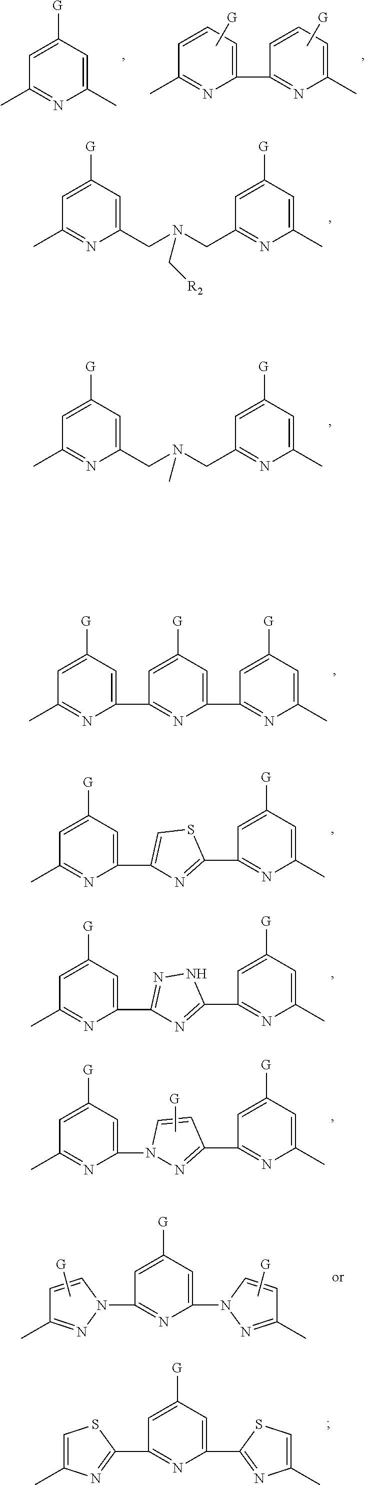
and wherein
G is in each case independently selected from i) a conjugating group, ii) a single bond, and iii) hydrogen;
wherein each conjugating group comprises 1, 2, or 3 moieties selected from —CH═CH—, —C≡C—, —C(═O)—, and biradicals of 5 to 10-membered aromatic or heteroaromatic monocyclic or bicyclic rings, wherein the heteroaromatic ring contains one or more, same or different heteroatoms N, O, or S, and wherein the aromatic or heteroaromatic monocyclic or bicyclic rings are unsubstituted or substituted by 1 to 5 same or different substituents R₅;
wherein each conjugating group, if present in a terminal position, may further comprise a terminal group, which is selected from the group consisting of —H, -halogen, —CN, —CH₃, and from the option of representing one of the one or two groups -L-Z;
wherein R₅ is independently selected from C₁-C₁₂-alkyl, —(CH₂)₀₋₆—C(═O)OH, —(CH₂)₀₋₆—C(═O)O⁻, —(CH₂)₀₋₆—S(═O)₂OH, —(CH₂)₀₋₆—S(═O)₂O⁻, —C(═O)NHR₆, —C(═O)NCH₃R₆, —NHC(═O)NHR₆, —NHC(═S)NHR₆, -halogen, —OH, —SH, —OR₇, —SR₇, and hydrophilic groups selected from monosaccharides, disaccharides, —(CH₂)₁₋₆ CH₂OH, —CH(CH₂OH)₂,
—C(CH₂OH)₃, —(CH₂)₁₋₃—O—(CH₂CH₂O)₀₋₅—H, —(CH₂)₁₋₃—O—(CH₂CH₂O)₀₋₅—C₁-C₄-alkyl, —O—(CH₂CH₂O)₁₋₆—H, and —O—(CH₂CH₂O)₁₋₆—C₁-C₄-alkyl;
wherein R₆ is selected from C₁-C₁₂-alkyl, —(CH₂)₁₋₆C(═O)OH, —(CH₂)₁₋₆C(═O)O⁻, —(CH₂)₁₋₆ S(═O)₂OH, —(CH₂)₁₋₆ S(═O)₂O⁻ and hydrophilic groups selected from monosaccharides, disaccharides, —(CH₂)₁₋₆ CH₂OH, —CH(CH₂OH)₂, —C(CH₂OH)₃, —(CH₂)₁₋₃—O—(CH₂CH₂O)₀₋₅—H, —(CH₂)₁₋₃—O—(CH₂CH₂O)₀₋₅—C₁-C₄-alkyl, —O—(CH₂CH₂O)₁₋₆—H, and —O—(CH₂CH₂O)₁₋₆—C₁-C₄-alkyl;
wherein R₇ is selected from —CF₃, —C₁-C₁₂-alkyl, —(CH₂)₁₋₆C(═O)OH, —(CH₂)₁₋₆C(═O)O⁻, —(CH₂)₁₋₆S(═O)₂OH, —(CH₂)₁₋₆S(═O)₂O⁻, —C(═O)NHR₆, —C(═O)NCH₃R₆, —NHC(═O)NHR₆,
—NHC(═S)NH R₆,
(CH₂)₁₋₆N(CH₃)₂⁺—(CH₂)₁₋₆S(═O)₂O⁻, —(CH₂)₁₋₆C(═O)-(piparazin-1,4-diyl)-(CH₂)₁₋₆C(═O)OH, and hydrophilic groups selected from monosaccharides, disaccharides, —(CH₂)₁₋₆ CH₂OH, —CH(CH₂OH)₂,
C(CH₂OH)₃, —(CH₂)₁₋₃—O—(CH₂CH₂O)₀₋₅—H, —(CH₂)₁₋₃—O—(CH₂CH₂O)₀₋₅—C₁-C₄-alkyl, —O—(CH₂CH₂O)₁₋₆—H, and —O—(CH₂CH₂O)₁₋₆—C₁-C₄-alkyl.

2. The compound of claim 1, wherein each conjugating group comprises the 1, 2 or 3 moieties in an arrangement so as to be conjugated with each other and attached to the respective pyridine in such a way that the conjugating group is conjugated with the pyridine.

3. The compound of claim 1, wherein each conjugating group comprises 1, 2, or 3 moieties selected from —CH═CH—, —C≡C—, —C(═O)—, phenylene, biphenylene, naphthylene, pyridylene, pyrazinylene, pyrimidinylene, pyridazinylene, furylene, thienylene, pyrrolylene, imidazolylene, pyrazolylene, thiazolylene, isothiazolylene, oxazolylene, isoxazolylene, fyrazanylene, 1,2,4-triazol-3,5-ylene, and oxadiazolylene, wherein the aromatic or heteroaromatic monocyclic or bicyclic rings are unsubstituted or substituted by 1 to 5 same or different substituents R₅.

4. The compound of claim 1, wherein
L is in each case independently absent or selected from linker groups comprising from 1 to 10 moieties selected from —(CH₂)₁₋₈ —, —CH═CH—, —C≡C—, —O—, —S—, —S—S—, —C(═O)—, —C(═O)NH—, —NHC(═O)—, —C(═O)N(C₁-C₆-alkyl)-, —N(C₁-C₆-alkyl)C(═O)—, —NHC(═S)NH—, —CH[(CH₂)₀₋₆C(═O)O⁻]—, —CH[(CH₂)₀₋₆C(═O)OH]—, phenylene, pyridylene, and triazolene.

5. The compound of claim 1, wherein
Z is in each case independently selected from reactive groups selected from —N₃, —C≡CH, —CH═CH₂, —NH₂, —O—NH₂, —C(═O)OH, —CH(═O), —SH, —OH, maleimido, —NCO, —NCS, —N⁺≡N, bromoacetamido, iodoacetamido, aromatic esters based on p-nitrophenol, pentafluorophenol, 2,4,5-trichlorophenol, N-hydroxy-5-norbornene-endo-2,3-dicarboxyimide, hydroxybenzotriazole, 1-hydroxy-7-azabenzoptriazole, sulfo-N-hydroxysuccinimide, or N-hydroxysuccinimide, esters based on phosphonium-, uronium-, or guanidinium-based coupling reagents, triazinyl or pyridinium esters, pyridyl-2-dithio, and 6-substituted 4-chloro-1,3,5-triazin-2-ylamino and 4-chloro-1,3,5-triazin-2-yloxy;
wherein the substituent in the 6-position of the 4-chloro-1,3,5-triazin-2-ylamino or 4-chloro-1,3,5-triazin-2-yloxy is selected from —H, -halogen, —SH, —NH₂, —C₁-C₆-alkyl, —O(C₁-C₆-alkyl), —OAryl, —S(C₁-C₈-alkyl), —SAryl, —N(C₁-C₈-alkyl)₂, and N(Aryl)₂;
wherein the carbon atoms of the aforementioned groups are unsubstituted or substituted by one or more substituents selected from —CN, -halogen, —SH, —C(═O)H, —C(═O)OH, C₁-C₆-alkyl, C₁-C₆-haloalkyl, —O(C₁-C₆-alkyl), —C(═O)(C₁-C₆-alkyl), —C(═O)O(C₁-C₆-alkyl), and phenyl.

6. The compound of claim 1, wherein A₁ is a bridging chelate moiety -Che₃-, wherein Ar₁ is the following group:

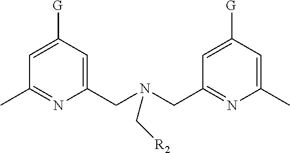

7. The compound of claim 1, wherein Che₁ and Che₂ are independently selected from the chelate moieties Che I and Che IV, wherein Ar₂ is the following group:

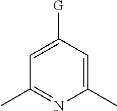

8. The compound of claim 1, which is any one of the compounds 6, 7, 26, 27, 28, 29, 35, 36, 45, 46, 47, 48, 56, 57, 58, 59, 65, and 66.

9. A compound of formula (II) 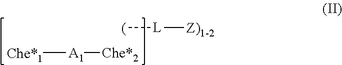
or a salt thereof, wherein
L is in each case independently absent or selected from linker groups comprising from 1 to 10 moieties selected from —(CH₂)₁₋₈—, —CH═CH—, —C≡C—, —O—, —S—, —S—S—, —C(═O)—, —C(═O)NH—, —NHC(═O)—, —C(═O)N(C₁-C₆-alkyl)-, —N(C₁-C₆-alkyl)C(═O)—, —NHC(═S)NH—, —CH[(CH₂)₀₋₆C(═O)O⁻]—, —CH[(CH₂)₀₋₆C(═O)OH]—, and biradicals of 5- to 10-membered aromatic or heteroaromatic monocyclic or bicyclic rings, wherein the heteroaromatic ring contains one or more, same or different heteroatoms N, O, or S;
Z is in each case independently selected from reactive groups selected from —N₃, —C≡CH, —CH═CH₂, —NH₂, —O—NH₂, —C(═O)OH, —CH(═O), —SH, —OH, maleimido and activated derivatives thereof including —NCO, —NCS, —N⁺≡N, bromoacetamido, iodoacetamido, reactive esters, pyridyl-2-dithio, and 6-substituted 4-chloro-1,3,5-triazin-2-ylamino and 4-chloro-1,3,5-triazin-2-yloxy;
wherein the substituent in the 6-position of the 4-chloro-1,3,5-triazin-2-ylamino or 4-chloro-1,3,5-triazin-2-yloxy is selected from —H, -halogen, —SH, —NH₂, —C₁-C₆-alkyl, —O(C₁-C₆-alkyl), —OAryl, —S(C₁-C₆-alkyl), —SAryl, —N(C₁-C₆-alkyl)₂, and N(Aryl)₂;
wherein the carbon atoms of the aforementioned groups are unsubstituted or substituted by one or more substituents selected from —CN, -halogen, —SH, —C(═O)H, —C(═O)OH, C₁-C₆-alkyl, C₁-C₆-haloalkyl, —O(C₁-C₆-alkyl), —C(═O)(C₁-C₆-alkyl), —C(═O)O(C₁-C₆-alkyl), and phenyl;
and wherein
A₁ is a bridging group comprising from one to three separate straight or branched, saturated or unsaturated carbon-based chains including from 1 to 12 carbon atoms, wherein the carbon-based chains are free of or comprise one to ten, same or different groups selected from —O—, —S—, —NH—, —NR₁—, —C(═O)NH—, —NHC(═O)—, —C(═O)NR₁—, —NR₁C(═O)— and —C(═O)—,
or
A₁ a bridging chelate moiety -Che*₃- of the following general formula 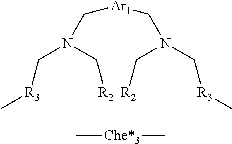
and wherein
Che*₁ and Che*₂ are independently selected from the chelate moieties Che* I, Che* II, Che* III, Che* IV, Che* V, Che* VI, and Che* VII of the following general formulae: 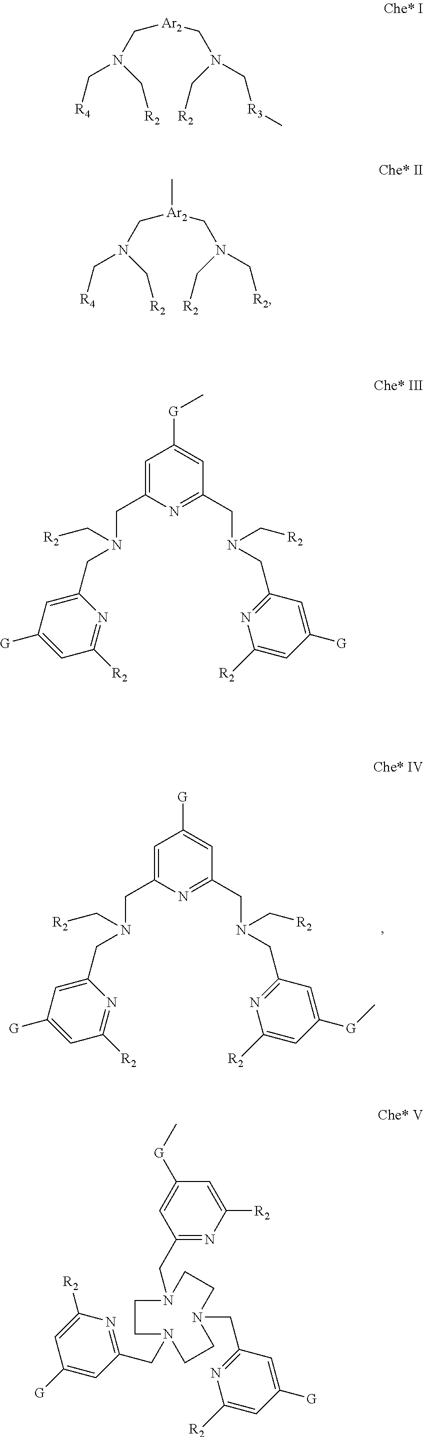 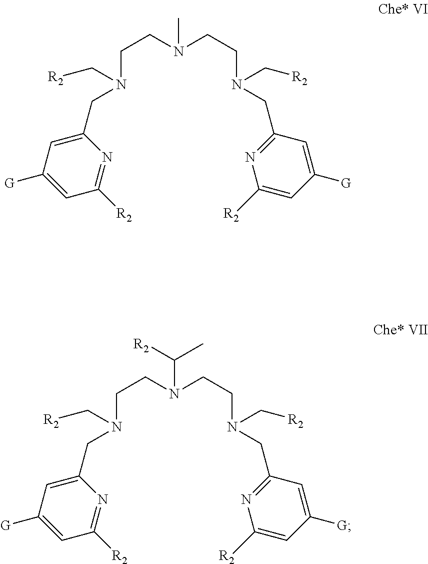
and wherein
R₁ is in each case independently selected from C₁-C₆-alkyl, and from the option of representing one of the one or two groups -L-Z;
R₂ is in each case independently selected from —C(═O)O⁻, —P(═O)O₂²⁻, P(═O)MeO⁻, —P(═O)PhO⁻, and the C₁-C₆-alkyl esters thereof, and from the option of representing one of the one or two groups -L-Z;
R₃ is in each case independently selected from the bridging groups
—C(═O)NH—, —C(═O)NH—(C₁-C₆-alkylene)-NHC(═O)—, —C(═O)NH—(C₁-C₆-alkylene)-O—, —C(═O)NH—(C₁-C₆-alkylene)-C(═O)NH—,
—C(═O)NH—(C₁-C₆-alkylene)-S—, —C(═O)NH—(C₁-C₆-alkylene)-NHC(═O)—(C₁-C₆-alkylene)-NHC(═O)—,
—C(═O)NH—(C₁-C₆-alkylene)-NHC(═O)—(C₁-C₆-alkylene)-O—, —C(═O)NH—(C₁-C₆-alkylene)-NHC(═O)—(C₁-C₆-alkylene)-C(═O)NH—, —C(═O)NH—(C₁-C₆-alkylene)-NHC(═O)—(C₁-C₆-alkylene)-S—, —C(═O)NH—(C₁-C₆-alkylene)-O—(C₁-C₆-alkylene)-NHC(═O)—,
—C(═O)NH—(C₁-C₆-alkylene)-O—(C₁-C₆-alkylene)-O—, —C(═O)NH—(C₁-C₆-alkylene)-O—(C₁-C₆-alkylene)-S—,
—C(═O)NH—(C₁-C₆-alkylene)-O—(C₁-C₆-alkylene)-C(═O)NH—, —C(═O)NH—(C₁-C₆-alkylene)-C(═O)NH—(C₁-C₆-alkylene)-NHC(═O)—, —C(═O)NH—(C₁-C₆-alkylene)-C(═O)NH—(C₁-C₆-alkylene)-O—, —C(═O)NH—(C₁-C₆-alkylene)-C(═O)NH—(C₁-C₆-alkylene)-S—, —C(═O)NH—(C₁-C₆-alkylene)-C(═O)NH—(C₁-C₆-alkylene)-C(═O)NH—, and —P(═O)O₂²⁻;
R₄ is in each case independently selected from
—CH₂N(CH₂C(═O)O⁻)₂, —CH₂N(CH₂P(═O)O₂²⁻)₂, —CH₂N(CH₂P(═O)MeO⁻)₂, —CH₂N(CH₂P(═O)PhO⁻)₂, and from the options defined for R₂;
Ar₁ is selected from the following groups 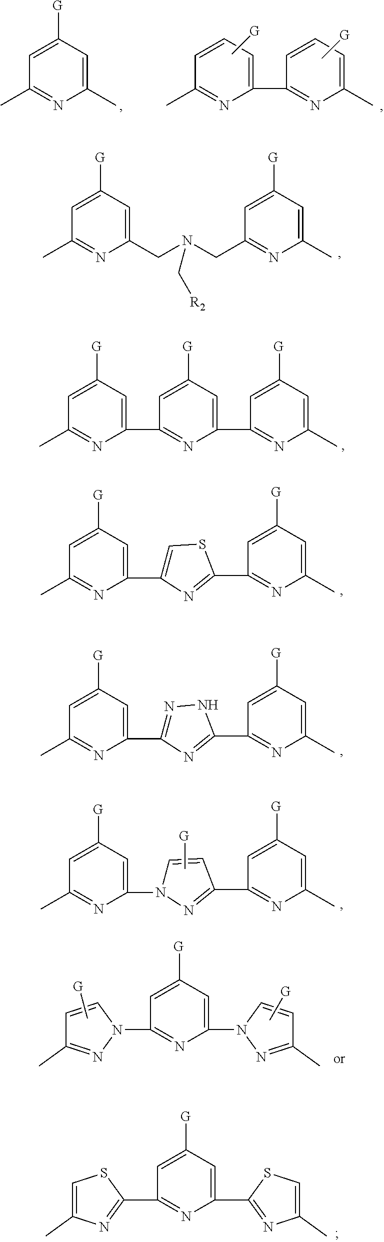
Ar₂ is in each case independently selected from the following groups 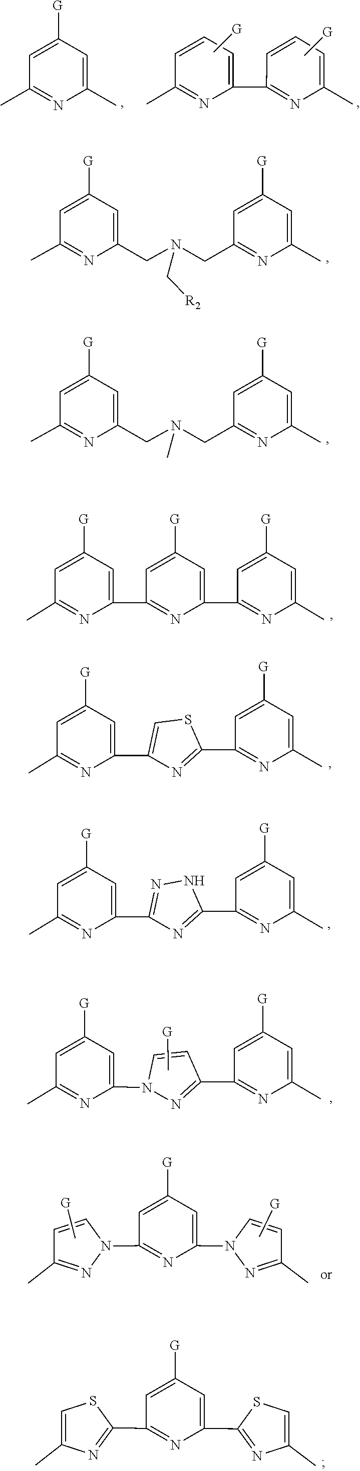
and wherein
G is in each case independently selected from i) a conjugating group, ii) a single bond, and iii) hydrogen;
wherein each conjugating group comprises 1, 2, or 3 moieties selected from —CH═CH—, —C≡C—, —C(═O)—, and biradicals of 5 to 10-membered aromatic or heteroaromatic monocyclic or bicyclic rings, wherein the heteroaromatic ring contains one or more, same or different heteroatoms N, O, or S, and wherein the aromatic or heteroaromatic monocyclic or bicyclic rings are unsubstituted or substituted by 1 to 5 same or different substituents R₅;
wherein each conjugating group, if present in a terminal position, may further comprise a terminal group, which is selected from the group consisting of —H, -halogen, —CN, —CH₃, and from the option of representing one of the one or two groups -L-Z;
wherein R₅ is independently selected from C₁-C₁₂-alkyl, —(CH₂)₀₋₆—C(═O)OH, —(CH₂)₀₋₆—C(═O)O⁻, —(CH₂)₀₋₆—S(═O)₂OH, —(CH₂)₀₋₆—S(═O)₂O⁻, —C(═O)NHR₆, —C(═O)NCH₃R₆, —NHC(═O)NHR₆, —NHC(═S)NHR₆, -halogen, —OH, —SH, —OR₇, —SR₇, and hydrophilic groups selected from monosaccharides, disaccharides, —(CH₂)₁₋₆CH₂OH, —CH(CH₂OH)₂,
—C(CH₂OH)₃, —(CH₂)₁₋₃—O—(CH₂CH₂O)₀₋₅—H, —(CH₂)₁₋₃—O—(CH₂CH₂O)₀₋₅—C₁-C₄-alkyl, —O—(CH₂CH₂O)₁₋₆—H, and —O—(CH₂CH₂O)₁₋₆—C₁-C₄-alkyl;
wherein R₆ is selected from C₁-C₁₂-alkyl, —(CH₂)₁₋₆C(═O)OH, —(CH₂)₁₋₆C(═O)O⁻, —(CH₂)₁₋₆S(═O)₂OH, —(CH₂)₁₋₆S(═O)₂O⁻ and hydrophilic groups selected from monosaccharides, disaccharides, —(CH₂)₁₋₆CH₂OH, —CH(CH₂OH)₂,
—C(CH₂OH)₃, —(CH₂)₁₋₃—O—(CH₂CH₂O)₀₋₅—H, —(CH₂)₁₋₃—O—(CH₂ CH₂O)₀₋₅—C₁-C₄-alkyl, —O—(CH₂CH₂O)₁₋₆—H, and —O—(CH₂CH₂O)₁₋₆—C₁-C₄-alkyl;
wherein R₇ is selected from —CF₃, —C₁-C₁₂-alkyl, —(CH₂)₁₋₆C(═O)OH, —(CH₂)₁₋₆C(═O)O⁻, (CH₂)₁₋₆S(═O)₂OH, —(CH₂)₁₋₆S(═O)₂O⁻, —C(═O)NHR₆, —C(═O)NCH₃R₆, —NHC(═O)NHR₆,
—NHC(═S)NH R₆,
—(CH₂)₁₋₆N(CH₃)₂⁺—(CH₂)₁₋₆S(═O)₂O⁻—, —(CH₂)₁₋₆C(═O)-(piparazin-1,4-diyl)-(CH₂)₁₋₆C(═O)OH, and hydrophilic groups selected from monosaccharides, disaccharides, —(CH₂)₁₋₆CH₂OH, —CH(CH₂OH)₂,
C(CH₂OH)₃, —(CH₂)₁₋₃—O—(CH₂CH₂O)₀₋₅—H, —(CH₂)₁₋₃—O—(CH₂CH₂O)₀₋₅—C₁-C₄-alkyl, —O—(CH₂CH₂O)₁₋₆—H, and —O—(CH₂CH₂O)₁₋₆—C₁-C₄-alkyl.

10. A detection agent comprising a biospecific binding reactant conjugated to a compound of claim 1.

11. The detection agent of claim 10, wherein the biospecific binding reactant is selected from
i) an antibody, an antigen, a receptor ligand, a specific binding protein, a DNA probe, a RNA probe, a hapten, a drug, and lectin; or
ii) an oligopeptide, an oligonucleotide, a modified oligonucleotide, a modified polynucleotide, a protein, an oligosaccaride, a polysaccharide, a phospholipid, a PNA and a steroid.

12. A method of detecting an analyte in a biospecific binding assay, said method comprising:
a) forming a complex between the analyte and a compound of claim 1;
b) exciting said complex with a radiation having an excitation wavelength of the compound, thereby forming an excited complex; and
c) detecting emission radiation emitted from said excited complex.

13. A method of labelling a biospecific binding reactant with a compound of claim 1, comprising:
a) providing a biospecific binding reactant; and
b) conjugating the biospecific binding reactant with the compound.

14-16. (canceled)

17. A solid support material conjugated with a compound of claim 1.

18. The compound of claim 3, wherein each conjugating group is independently selected from phenylene-C≡C—, phenylene, thienylene, and furylene.

19. The compound of claim 4, wherein:
L is in each case independently absent or selected from linker groups comprising from 1 to 10 moieties selected from —(CH₂)₁₋₈ —, —C≡C—, —O—, —C(═O)—, —C(═O)NH—, —NHC(═O)—, phenylene, pyridylene, and triazolene.

20. The compound of claim 5, wherein:
Z is in each case independently —NCS or —NH₂.

21. The compound of claim 6, wherein:
R₂ is in each case —C(═O)O⁻; and
R₃ is in each case selected from the bridging groups —C(═O)NH—, —C(═O)NH—(C₁-C₆-alkylene)-NHC(═O)—, —C(═O)NH—(C₁-C₆-alkylene)-O—, —C(═O)NH—(C₁-C₆-alkylene)-C(═O)NH—, —C(═O)NH—(C₁-C₆-alkylene)-S—.

22. The compound of claim 7, wherein:
R₂ is in each case —C(═O)O⁻;
R₃ is in each case selected from the bridging groups —C(═O)NH—, —C(═O)NH—(C₁-C₆-alkylene)-NHC(═O)—, —C(═O)NH—(C₁-C₆-alkylene)-O—, —C(═O)NH—(C₁-C₆-alkylene)-C(═O)NH—, —C(═O)NH—(C₁-C₆-alkylene)-S—; and
R₄ is in each case —C(═O)O⁻.

23. The detection agent of claim 11, wherein the biospecific binding reactant is an antibody.
